(19) United States
(12) Patent Application Publication
(10) Pub. No.: US 2005/0085447 A1
(43) Pub. Date: Apr. 21, 2005

(54) Pyrido[3,4-d]pyrimidine derivatives as matrix metalloproteinase-13 inhibitors

(75) Inventors: Amy Mae Bunker, Ann Arbor, MI (US); Joseph Armand Picard, Canton, MI (US); Rita Mayur Lodaya, Northville, MI (US); Michael Lane Waldo, Canton, MI (US); Mark Eugene Marlatt, Grass Lake, MI (US)

Correspondence Address: WARNER-LAMBERT COMPANY, 2800 PLYMOUTH RD, ANN ARBOR, MI, 48105, US

(73) Assignee: Warner-Lambert Company LLC

(21) Appl. No.: 10/921,055

(22) Filed: Aug. 18, 2004

Related U.S. Application Data
(60) Provisional application No. 60/496,160, filed on Aug. 19, 2003.

(51) Int. Cl. A61K 31/69; C07F 5/02

(52) U.S. Cl. 514/64; 544/229

(57) ABSTRACT

This invention relates to a pyrido[3,4-d]pyrimidine derivative of Formula I 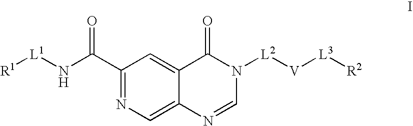
or a pharmaceutically acceptable salt thereof, wherein R¹, L¹, L², V, L³, and R² are as defined in the specification, that inhibits a matrix metalloproteinase-13 enzyme and thus is useful for treating diseases resulting from MMP-13 mediated tissue breakdown such as osteoarthritis, rheumatoid arthritis, cartilage damage, psoriatic arthritis, ankylosing spondylitis, heart failure, atherosclerosis, inflammatory bowel disease, multiple sclerosis, age-related macular degeneration, chronic obstructive pulmonary disease, asthma, periodontal diseases, psoriasis, cancer, and osteoporosis.

### CROSS-REFERENCE TO RELATED APPLICATIONS

This application claims benefit of U.S. Provisional Patent Application No. 60/496,160, filed Aug. 19, 2003.

### FIELD OF THE INVENTION

This invention relates to pyrido[3,4-d]pyrimidine derivatives that inhibit a matrix metalloproteinase-13 enzyme and thus are useful for treating diseases resulting from MMP-13 mediated tissue breakdown such as osteoarthritis, rheumatoid arthritis, cartilage damage, psoriatic arthritis, ankylosing spondylitis, heart failure, atherosclerosis, inflammatory bowel disease, multiple sclerosis, age-related macular degeneration, chronic obstructive pulmonary disease, asthma, periodontal diseases, psoriasis, cancer, and osteoporosis.

### BACKGROUND OF THE INVENTION

Matrix metalloproteinases (sometimes referred to as MMPs) are naturally occurring enzymes found in most mammals. Over-expression and activation of MMPs, or an imbalance between MMPs and endogenous inhibitors of MMPs (i.e., tissue inhibitors of matrix metalloproteinases or “TIMPs”), have been suggested as factors in the pathogenesis of diseases characterized by the breakdown of extracellular matrix or connective tissues.

Pathological imbalance or over-expression and activation of matrix metalloproteinase-13 (“MMP-13”) has been directly implicated in diseases such as, for example, osteoarthritis, rheumatoid arthritis, cartilage damage, abdominal aortic aneurysms, heart failure, skin ulcers, and metastasis or angiogenesis of a cancer selected from the group consisting of: ovarian cancer, squamous carcinoma, head carcinoma, neck carcinoma, fibrosarcoma, chondrosarcoma, basal cell carcinoma of the skin, and breast cancer.

Selective inhibitors of MMP-13 include a compound named WAY-170523, which has been reported by Chen et al., J. Am. Chem. Soc., 2000; 122:9648-9654 and other compounds are reported in PCT International Patent Application Publication numbers WO 00/09485; WO 01/12611; WO 01/63244; WO 02/34726; WO 02/34753; WO 02/064547; WO 02/064598; WO 02/064080; WO 02/064572; WO 02/064595; WO 02/064578; WO 02/064571; and WO 02/064568, and their corresponding U.S. patent application publication Nos. US2002-0156061; US2003-0004172; US2003-0078276; US2002-0193377; US2002-0151558; US2002-0156069; US2002-0151555; and US2002-0161000, respectively, and PCT International Patent Application Publication numbers WO 02/064599 and WO 03/032999, and European Patent Application numbers EP 935,963 and EP 1,138,680. Further, U.S. patent application publication No. 2003-0087924 and U.S. Pat. No. 6,008,243 disclose inhibitors of MMP-13.

However, no inhibitor of MMP-13 has been approved and marketed for the treatment of any disease in any mammal. Accordingly, the need continues to find new low molecular weight compounds that are potent and selective inhibitors of MMP-13 over one or more MMP enzymes, including MMP-1, MMP-2, MMP-3, MMP-7, MMP-9, MMP-12, MMP-14, and/or MMP-17. These compounds will ideally be characterized by an acceptable therapeutic index of toxicity/potency that allows them to be used clinically for the prevention and treatment of MMP-13 associated disease states in a human or other mammals. An object of this invention is to provide said potent and specific inhibitors of MMP-13 that are characterized as being pyrido[3,4-d]pyrimidine derivatives.

### SUMMARY OF THE INVENTION

There are many aspects of the present invention. One aspect of this invention is a pyrido[3,4-d]pyrimidine derivative of Formula I 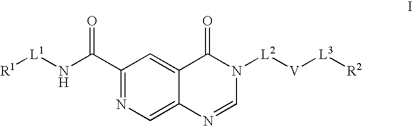
or a pharmaceutically acceptable salt thereof,
wherein:
R¹ is a radical independently selected from the group consisting of phenyl, naphthyl, 5- and 6-membered heteroaryl, and 9- and 10-membered heterobiaryl, wherein said R¹ radicals are unsubstituted or substituted with from 1 to 4 substituents R^{X};
L¹ is a diradical independently selected from the group consisting of CH₂, CH₂CH₂, OCH₂, N(H)CH₂, S(O)₂, CH₂S(O)₂, SCH₂, S(O)CH₂, and S(O)₂CH₂, wherein said L¹ diradicals are unsubstituted or substituted with 1 or 2 substituents R^{X};
L² is a diradical independently selected from the group consisting of CH₂, S(O), S(O)₂, CH₂CH₂, CH₂O, CH₂N(H), CH₂S, CH₂S(O), S(O)CH₂, CH₂S(O)₂, and S(O)₂CH₂, wherein said L² diradicals are unsubstituted or substituted with 1 or 2 substituents R^{X};
V is a diradical independently selected from the group consisting of phenylene, naphthylene, 5- and 6-membered heteroarylene, 9- and 10-membered heterobiarylene, C₃-C₆ cycloalkylene, 3- to 6-membered heterocycloalkylene, C₆-C₁₀ bicycloalkylene, and 6- to 10-membered heterobicycloalkylene, wherein said V diradicals are unsubstituted or substituted with from 1 to 4 substituents R^{X};
L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂ wherein said L³ diradicals are unsubstituted or substituted with 1 or 2 substituents R^{X};
R² is a radical independently selected from the group consisting of —SO₃H, —PO₃H₂, —(CH₂)_{0 or 1}—N(H)-G-R, —C(O)N(H)-G-R, -G-N(H)—C(O)—R, and a 5-membered heterocycle radical selected from the group consisting of: 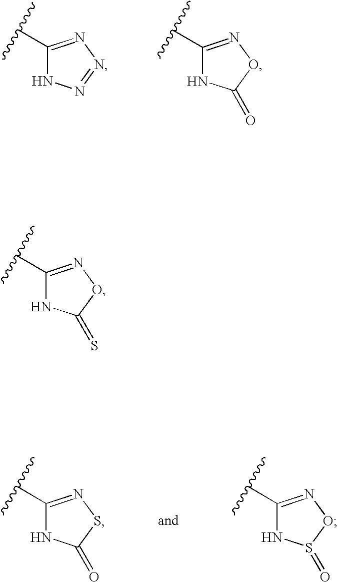
   and
R² may further be a radical independently selected from the group consisting of —CO₂H, when L³ is absent or is a diradical independently selected from the group consisting of CH₂, CH₂CH₂, OCH₂, N(H)CH₂, SCH₂, S(O)CH₂, S(O)₂CH₂, wherein said L³ diradicals are unsubstituted or substituted with 1 or 2 substituents R^{X};
R is C₁-C₆ alkyl, wherein said C₁-C₆ alkyl is unsubstituted or substituted with from 1 to 4 substituents R^{X}; or
V, L³, and R² may be taken together to form a heterocycle radical selected from the group consisting of: 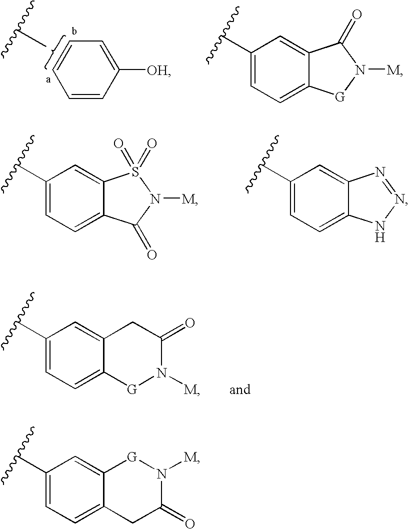
wherein said heterocycle radical is unsubstituted or substituted with from 1 to 3 groups R^{X};
G is C(O) or S(O)₂;
M is H or OH;
[symbol] indicates a radical point of attachment, which may be further indicated with a bracket { and letters a and b;
Each said R^{X} substituent, whether on a carbon or nitrogen atom, is independently selected from the group consisting of:
   C₁-C₆ alkyl; 2- to 6-membered heteroalkyl; C₃-C₅ cycloalkyl; 3- to 5-membered heterocycloalkyl, wherein said C₁-C₆ alkyl, 2- to 6-membered heteroalkyl, C₃-C₅ cycloalkyl, and 3- to 5-membered heterocycloalkyl are unsubstituted or substituted with from 1 to 3 groups independently selected from the group consisting of F, 2F, 3F, HO—, O═, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, and CH₃S(O)₂N(CH₃)—; (C₁-C₆ alkyl)-C(O), (C₁-C₆ alkyl)-S(O)_{1 or 2};
   H₂NS(O)₂—; (C₁-C₆ alkyl)-N(H)S(O)₂—; (C₁-C₆ alkyl)₂—NS(O)₂—; phenyl; 5-membered heteroaryl; and 6-membered heteroaryl, wherein said phenyl, 5-membered heteroaryl, and 6-membered heteroaryl are unsubstituted or are independently substituted on a carbon atom with from 1 to 3 groups selected from the group consisting of F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O)O—, CH₃S(O)₂, CH₃S(O)—, CH₃S(O)₂N(H)—, CH₃S(O)₂N(CH₃)—, and ═O,
   wherein said ═O is on a carbon atom that is contiguous to a nitrogen atom, and said 5-membered heteroaryl may also be optionally substituted on a nitrogen atom with CH₃;
wherein each substituent R^{X} on a carbon atom may further be independently selected from the group consisting of: (C₁-C₆ alkyl)-O, (C₁-C₆ alkyl)-S, H₂N, (C₁-C₆ alkyl)-N(H)—, (C₁-C₆ alkyl)₂N—, (C₁-C₆ alkyl)-C(O)O—, (Cₗ-C₆ alkyl)-C(O)N(H)—, HO, F, Cl, Br, I, and HO₂C; and
wherein two substituents R^{X} on the same carbon atom may be taken together with the carbon atom to which they are both bonded to form the group C═O;
wherein two adjacent substituents R^{X}, bonded to contiguous carbon atoms, may be taken together to form the diradical group —O—CH₂—O—;
wherein each unsubstituted C₁-C₆ alkyl is independently an acyclic hydrocarbon radical containing from 1 to 6 carbon atoms in a straight or branched configuration;
wherein each unsubstituted 2- to 6-membered heteroalkyl is independently an acyclic radical in a straight or branched configuration containing one heteroatom selected from the group consisting of O, S, S(O), S(O)₂, N, and N(H) and from 1 to 5 carbon atoms, respectively;
wherein each unsubstituted C₃-C₅ cycloalkyl is independently a monocyclic hydrocarbon radical containing from 3 to 5 carbon atoms;
wherein each unsubstituted 3- to 5-membered heterocycloalkyl is independently a monocyclic radical containing one heteroatom selected from the group consisting of O, S, S(O), S(O)₂, N, and N(H) and from 2 to 4 carbon atoms, respectively;
wherein each unsubstituted 5-membered heteroaryl is independently an aromatic monocyclic radical that contains carbon atoms and from 1 to 4 heteroatoms independently selected from the group consisting of 1 O, 1 S, 1 N(H), and 3 N, and does not contain an O atom contiguous to an S atom;
wherein each unsubstituted 6-membered heteroaryl is independently an aromatic monocyclic radical that contains carbon atoms and 1 or 2 nitrogen atoms;
wherein each unsubstituted 9- and 10-membered heterobiaryl is independently a [4.3.0] or [4.4.0] bicyclic radical, respectively, that contains carbon atoms and from 1 to 4 heteroatoms independently selected from the group consisting of 1 O, 1 S, 1 N(H), and 4 N, such that a 6-membered ring is fused to a 5-membered or 6-membered ring, respectively, wherein at least one of the two fused rings of the bicyclic radical is aromatic, wherein the bicyclic radical does not contain an O atom contiguous to an S atom;
wherein unsubstituted phenylene is 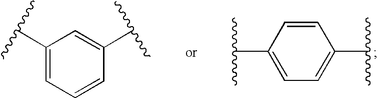
wherein unsubstituted naphthylene is 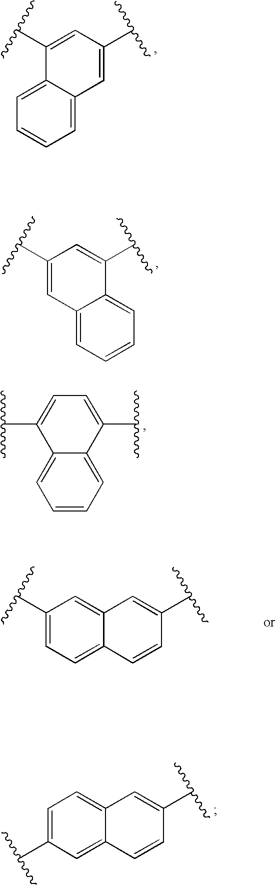
wherein an unsubstituted 5-membered heteroarylene is an aromatic monocyclic diradical that contains carbon atoms and from 1 to 4 heteroatoms independently selected from the group consisting of 1 O, 1 S, 1 N(H), and 3 N, and does not contain an O atom contiguous to an S atom, wherein the radicals do not reside on the same or adjacent ring atoms;
wherein an unsubstituted 6-membered heteroarylene is an aromatic monocyclic diradical that contains carbon atoms and 1 or 2 nitrogen atoms, wherein the radicals do not reside on the same or adjacent ring atoms;
wherein an unsubstituted 9- or 10-membered heterobiarylene is a [4.3.0] or [4.4.0] bicyclic diradical that contains carbon atoms and from 1 to 4 heteroatoms independently selected from the group consisting of 1 O, 1 S, 1 N(H), and 4 N, such that a 6-membered ring is fused to a 5-membered or 6-membered ring, respectively, wherein at least one of the two fused rings of the bicyclic diradical is aromatic, wherein the bicyclic diradical does not contain an O atom contiguous to an S atom, and wherein the radicals do not reside on the same or adjacent ring atoms or on ring atoms that are common to both rings;
wherein an unsubstituted C₃-C₆ cycloalkylene is a diradical containing from 3 to 6 carbon atoms and optionally containing 1 double bond, wherein the radicals do not reside on the same ring atom and when said C₃-C₆ cycloalkylene has from 4 to 6 carbon atoms, the radicals do not reside on adjacent ring atoms;
wherein an unsubstituted 3- to 6-membered heterocycloalkylene is a diradical as defined above for C₃-C₆ cycloalkylene, respectively, except wherein one of the carbon atoms of said C₃-C₆ cycloalkylene is replaced by a heteroatom selected from the group consisting of O, S, S(O), S(O)₂, N, and N(H);
wherein an unsubstituted C₆-C₁₀ bicycloalkylene is a fused or bridged bicyclic diradical containing from 6 to 10 carbon atoms, and optionally containing one double bond, wherein the radicals do not reside on the same or adjacent ring atoms; and
wherein an unsubstituted 6- to 10-membered heterobicycloalkylene is a diradical as defined above for C₆-C₁₀ bicycloalkylene, respectively, except wherein one of the carbon atoms of said C₆-C₁₀ bicycloalkylene is replaced by a heteroatom selected from the group consisting of O, S, S(O), S(O)₂, N, and N(H).

Another aspect of this invention is a compound of Formula II 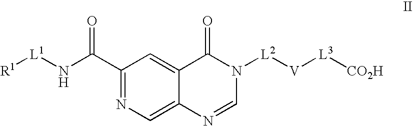
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, wherein said L³ diradicals are unsubstituted or substituted with 1 or 2 substituents R^{X}, wherein R^{X} is as defined above for Formula I.

Another aspect of this invention is a compound of Formula III 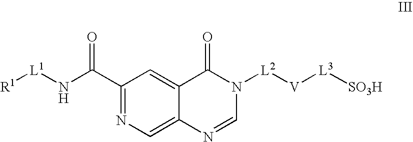
or a pharmaceutically acceptable salt thereof, wherein R¹, L², L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula IV 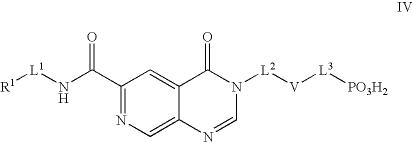
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula V 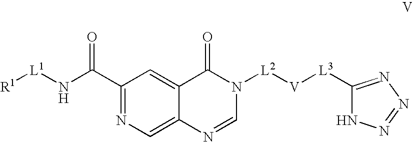
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula VI 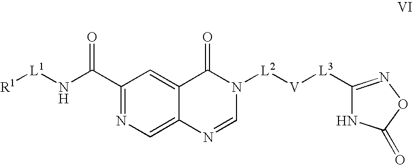
or a pharmaceutically acceptable salt thereof, wherein R¹, L², L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula VII 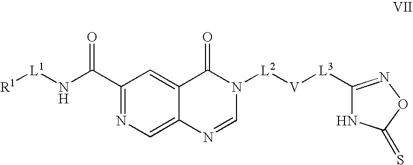
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula VIII 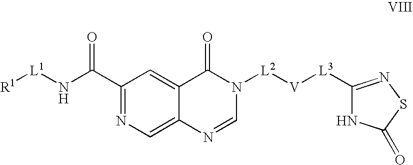
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula IX 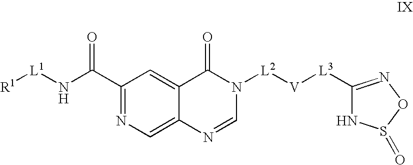
or a pharmaceutically acceptable salt thereof, wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, O, N(H), S, S(O), S(O)₂, CH₂O, CH₂N(H), CH₂S, CH₂S(O), and CH₂S(O)₂, wherein said L³ diradicals are unsubstituted or substituted with 1 or 2 substituents R^{X}, wherein R^{X} is as defined above for Formula I.

Another aspect of this invention is a compound of Formula X 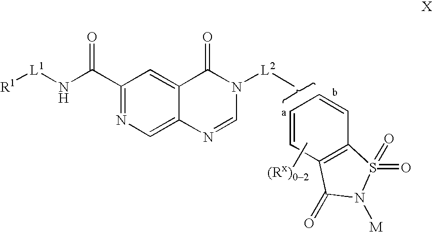
or a pharmaceutically acceptable salt thereof, wherein −{ indicates a radical point of attachment, which is further indicated with letters a and b, and R¹, L¹, L², M, and R^{X} are as defined above for Formula I.

Another aspect of this invention is a compound of Formula XI 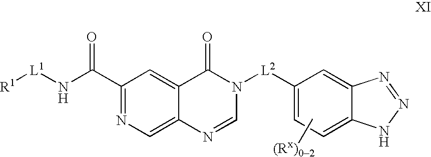
or a pharmaceutically acceptable salt thereof, wherein R¹, L¹, L², and R^{X} are as defined above for Formula I.

Another aspect of this invention is a compound of any one of Formulas I-XI, or a pharmaceutically acceptable salt thereof, wherein R¹ is selected from the group consisting of any one of groups (i)-(xi): (i) phenyl, 5- and 6-membered heteroaryl, and 9- or 10-membered heterobiaryl; (ii) phenyl, 5- and 6-membered heteroaryl, and 9-membered heterobiaryl; (iii) phenyl, and 5- and 6-membered heteroaryl, (iv) phenyl; (v) 5- and 6-membered heteroaryl, (vi) 5-membered heteroaryl; (vii) 6-membered heteroaryl; (viii) each of (i)-(vii) unsubstituted; (ix) each of (i)-(vii) optionally substituted with from 1 to 3 substituents R^{X}; (x) each of (i)-(vii) and (ix) optionally substituted with from 1 to 3 substituents R^{X} wherein R^{X} is independently CH₃O, F, Cl, CF₃, or CH₃; and (xi) each of (i)-(vii), (ix), and (x) optionally substituted with from 1 to 4 substituents R^{X} wherein R^{X} is independently CH₃O, F, Cl, CF₃, or CH₃ in the meta or para position relative to the attachment of R¹ to L¹.

Another aspect of this invention is a compound of any one of Formulas I-XI, or a pharmaceutically acceptable salt thereof, wherein L¹ is selected from the group consisting of any one of groups (i)-(xiv): (i) CH₂; (ii) CH₂CH₂, OCH₂, N(H)CH₂, SCH₂, S(O)CH₂, and S(O)₂CH₂; (iii) CH₂CH₂, OCH₂, and N(H)CH₂; (iv) CH₂CH₂; (v) OCH₂, and N(H)CH₂; (vi) OCH₂; (vii) N(H)CH₂; (viii) SCH₂, S(O)CH₂, and S(O)₂CH₂; (ix) SCH₂; (x) S(O)₂ and CH₂S(O)₂; (xi) S(O)CH₂ and S(O)₂CH₂; (xii) each of (i)-(xi) unsubstituted; (xiii) each of (i)-(xi) substituted with 1 or 2 substituents R^{X}; and (xiv) each of (i)-(xi) and (xiii) substituted with 1 or 2 substituents R^{X} wherein R^{X} is independently 2F, CH₃, or ═O.

Another aspect of this invention is a compound of any one of Formulas I-XI, or a pharmaceutically acceptable salt thereof, wherein L² is selected from the group consisting of any one of groups (i)-(xx): (i) CH₂, S(O), and S(O)₂; (ii) CH₂CH₂, CH₂O, CH₂N(H), CH₂S, S(O)CH₂, CH₂S(O), S(O)₂CH₂ and CH₂S(O)₂; (iii) CH₂; (iv) S(O); (v) S(O)₂; (vi) CH₂CH₂, CH₂O, and CH₂N(H); (vii) CH₂CH₂; (viii) CH₂O; (ix) CH₂N(H); (x) CH₂S, S(O)CH₂, CH₂S(O), S(O)₂CH₂ and CH₂S(O)₂; (xi) CH₂S; (xii) S(O)CH₂ and CH₂S(O); (xiii) S(O)CH₂; (xiv) CH₂S(O); (xv) S(O)₂CH₂ and CH₂S(O)₂; (xvi) S(O)₂CH₂; (xvii) CH₂S(O)₂; (xviii) each of (i)-(xvii) unsubstituted; (xix) each of (i)-(xvii) substituted with 1 or 2 substituents R^{X}; and (xx) each of (i)-(xvii) and (xix) substituted with 1 or 2 substituents R^{X} wherein R^{X} is independently 2F, CH₃, or ═O.

Another aspect of this invention is a compound of any one of Formulas I-XI, or a pharmaceutically acceptable salt thereof, wherein V is selected from the group consisting of any one of groups (i)-(xvi): (i) phenylene, 5- and 6-membered heteroarylene, C₃-C₆ cycloalkylene, and 3- to 6-membered heterocycloalkylene; (ii) phenylene, 5- and 6-membered heteroarylene, C₃-C₆ cycloalkylene, and 5- to 6-membered heterocycloalkylene; (iii) phenylene, 5- and 6-membered heteroarylene, C₅-C₆ cycloalkylene and 6-membered heterocycloalkylene; (iv) phenylene, 5- and 6-membered heteroarylene, and C₆ cycloalkylene; (v) phenylene, 6-membered heteroarylene, and C₆ cycloalkylene; (vi) phenylene and 6-membered heteroarylene; (vii) phenylene; (viii) 6-membered heteroarylene; (ix) C₆ cycloalkylene; (x) 6-membered heterocycloalkylene; (xi) 5-membered heteroarylene; (xii) naphthalene, 9- and 10-membered heterobiarylene, C₆-C₁₀ bicycloalkylene; and 6- to 10-membered heterobicycloalkylene; (xiii) naphthalene, 9- and 10-membered heterobiarylene; (xiv) each of (i)-(xiii) unsubstituted; (xv) each of (i)-(xiii) substituted with from 1 to 4 substituents R^{X}; and (xvi) each of (i)-(xiii) and (xv) substituted with from 1 to 4 substituents R^{X} wherein R^{X} is F or 2F.

In another aspect of this invention is a compound of any one of Formulas I-IX, or a pharmaceutically acceptable salt thereof, wherein V is a 5-membered heteroarylene selected from the group consisting of: 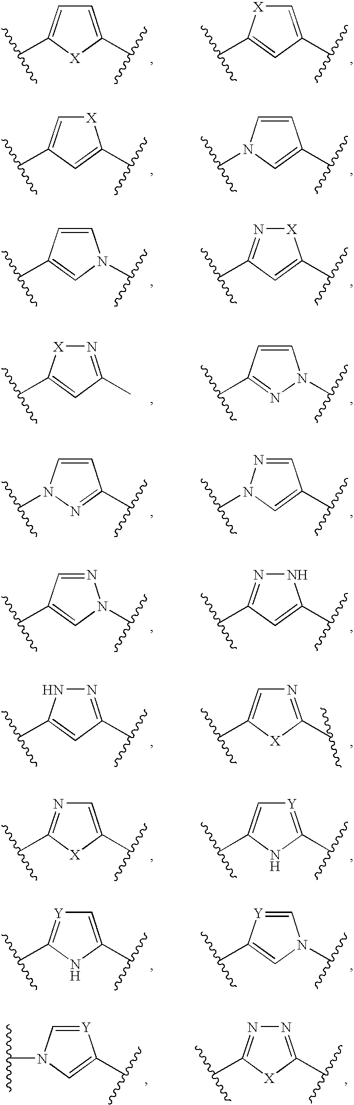 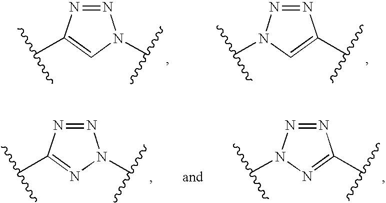
wherein X is O, S, or N(H), Y is O, S, or N, and the diradicals are unsubstituted or substituted with 1 or 2 substituents R^{X}.

Another aspect of this invention is a compound of any one of Formulas I-IX, or a pharmaceutically acceptable salt thereof, wherein V is a 6-membered heteroarylene selected from the group consisting of: 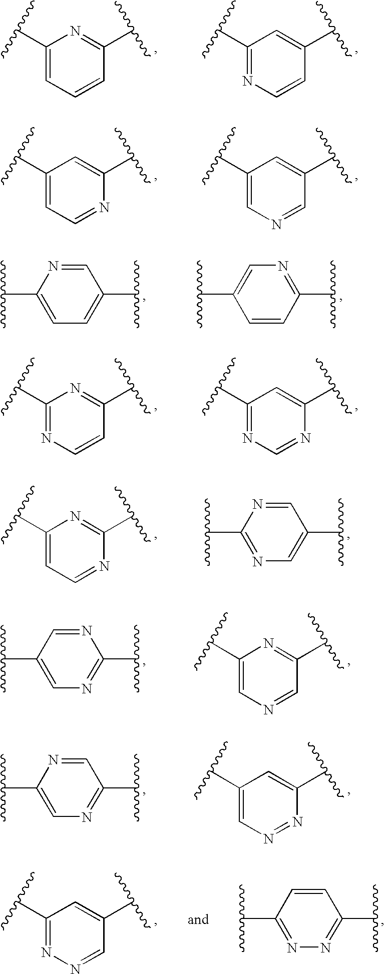
wherein the diradicals are unsubstituted or substituted with from 1 to 3 substituents R^{X}.

Another aspect of this invention is a compound of any one of Formulas I-IX, or a pharmaceutically acceptable salt thereof, wherein V is a 9-membered heterobiarylene selected from the group consisting of: 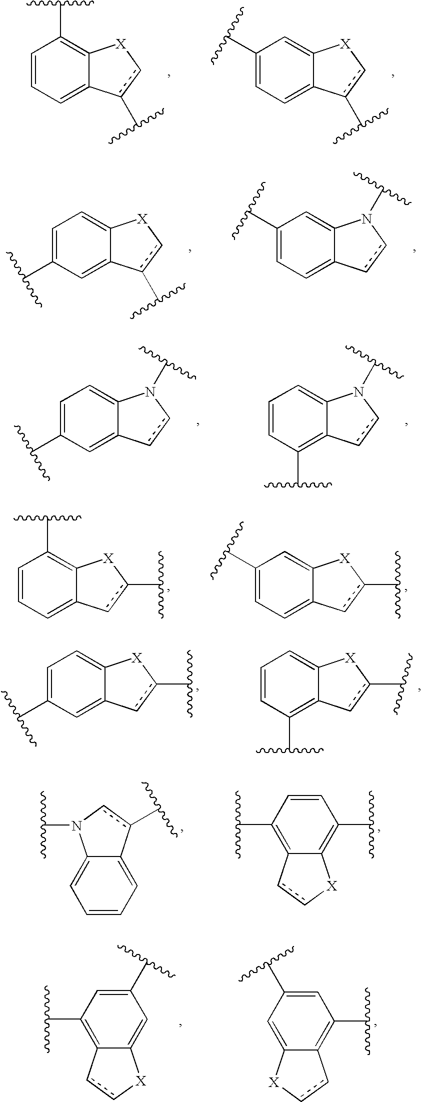 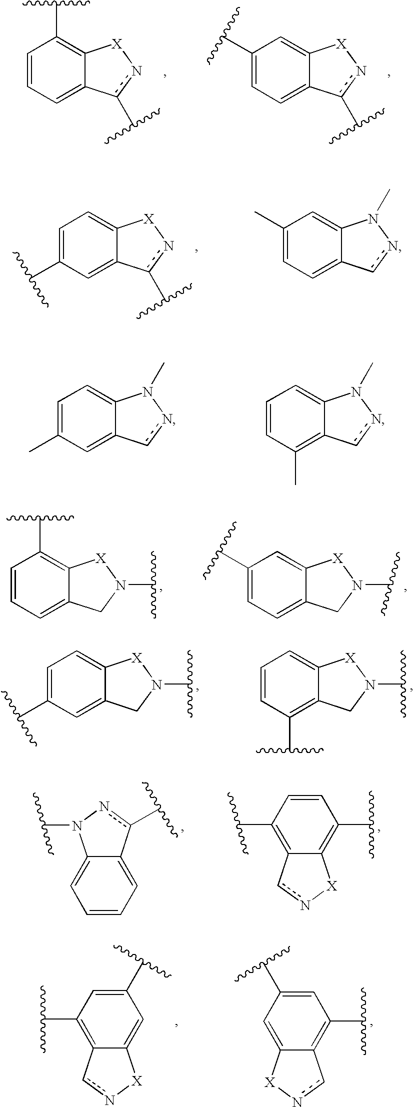 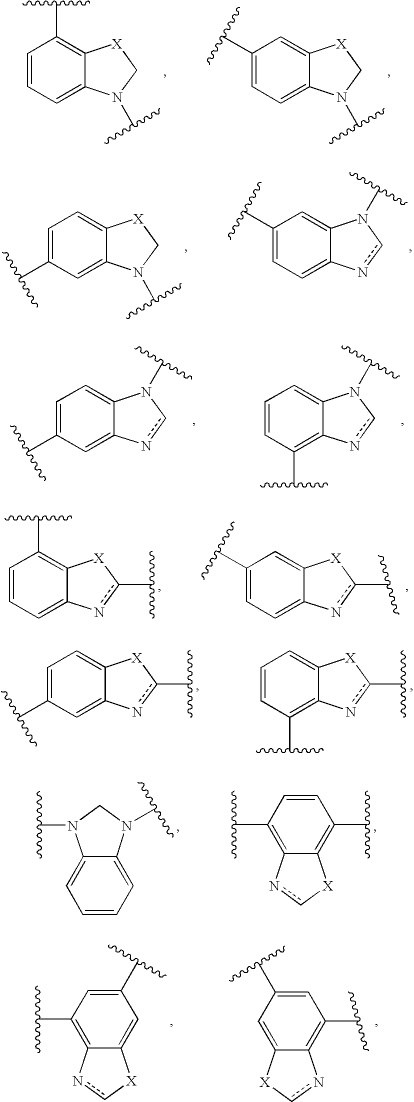 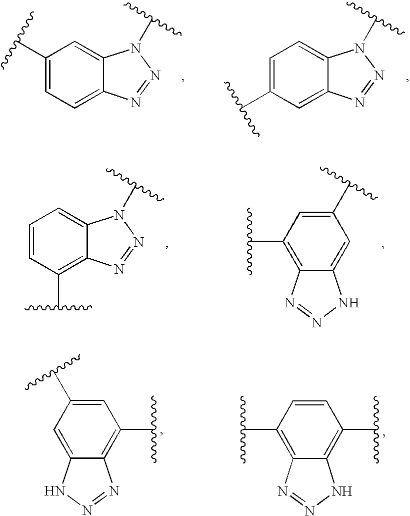
and the corresponding 9-membered heterobiarylenes wherein a C—H in the 6-membered benzo ring is replaced with N,
wherein X is O, S, or N(H), ---- is a pi-bond or is absent, and the diradicals are unsubstituted or substituted with from 1 to 4 substituents R^{X}.

Another aspect of this invention is a compound of any one of Formulas I-IX, or a pharmaceutically acceptable salt thereof, wherein V is a 10-membered heterobiarylene selected from the group consisting of: 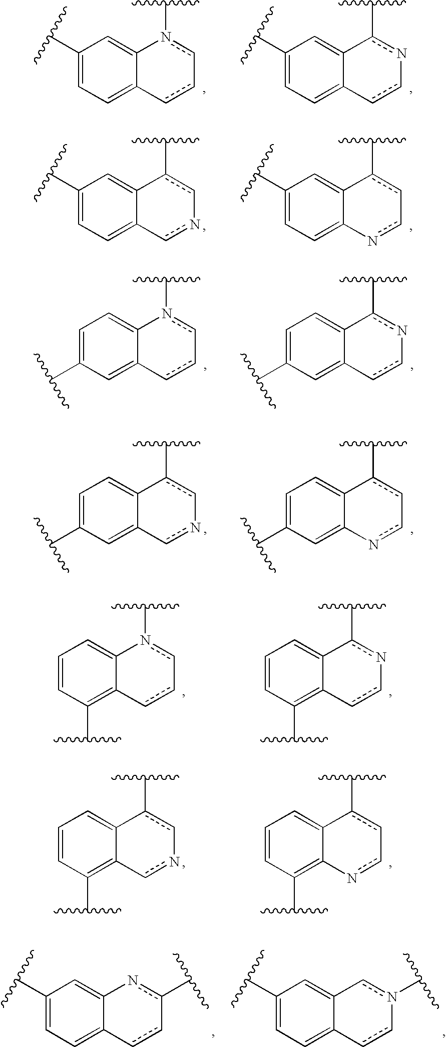 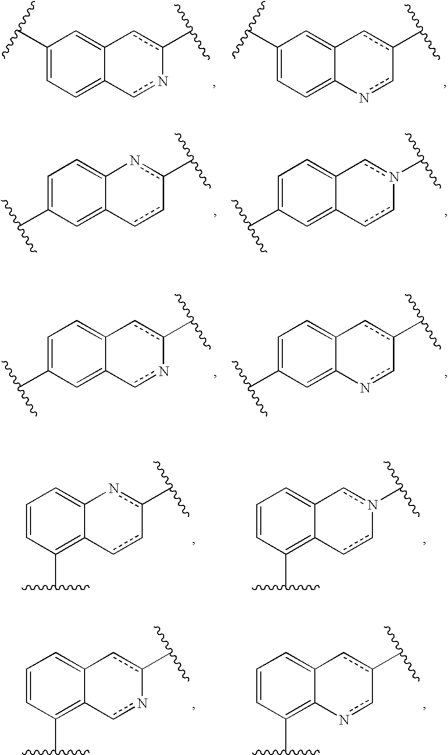
10-membered heterobiarylenes as drawn above and then horizontally flipped 180°, all of the previously described 10-membered heterobiarylenes wherein one C—H (i.e., sp² carbon atom) is replaced with N, and all of the previously described 10-membered heterobiarylenes wherein one CH₂ (i.e., sp³ carbon atom) is replaced with N(H),
wherein each ---- is independently a pi-bond or is absent, and the diradicals are unsubstituted or substituted with from 1 to 4 substituents R^{X}.

Another aspect of this invention is a compound of any one of Formulas I-IX, or a pharmaceutically acceptable salt thereof, wherein V is a C₃-C₆ cycloalkylene selected from the group consisting of trans-cycloprop-1,2-diyl, trans-cyclobut-1,3-diyl, trans-cyclopent-1,3-diyl, trans-cyclohex-1,4-diyl, cis-cycloprop-1,2-diyl, cis-cyclobut-1,3-diyl, cis-cyclopent-1,3-diyl, and cis-cyclohex-1,4-diyl, and the diradicals are unsubstituted or substituted with from 1 to 4 substituents R^{X}. Still another aspect of this invention is a C₃-C₆ cycloalkylene selected from the group consisting of trans-cycloprop-1,2-diyl, trans-cyclobut-1,3-diyl, trans-cyclopent-1,3-diyl, and trans-cyclohex-1,4-diyl.

Another aspect of this invention is a compound of any one of Formulas I-IX, or a pharmaceutically acceptable salt thereof, wherein V is a 3- to 6-membered heterocycloalkylene selected from the group consisting of aziridin-1,2-diyl, 1-azacyclobut-1,3-diyl, pyrrollidin-1,3-diyl, morpholin-2,4-diyl, thiomorpholin-2,4-diyl, piperidin-1,4-diyl, and piperizin-1,4-diyl, and the diradicals are unsubstituted or substituted with from 1 to 4 substituents R^{X}.

Another aspect of this invention is a compound of any one of Formulas I-IX, or a pharmaceutically acceptable salt thereof, wherein L³ is selected from the group consisting of any one of groups (i)-(xxviii): (i) absent and CH₂; (ii) CH₂; (iii) absent; (iv) CH₂CH₂, OCH₂, and N(H)CH₂; (v) CH₂CH₂; (vi) OCH₂; (vii) N(H)CH₂; (viii) SCH₂, S(O)CH₂, and S(O)₂CH₂; (ix) SCH₂; (x) S(O)CH₂; (xi) S(O)₂CH₂; (xii) O and N(H); (xiii) O; (xiv) N(H); (xv) S, S(O), and S(O)2; (xvi) S; (xvii) S(O); (xviii) S(O)₂; (xix) CH₂O and CH₂N(H); (xx) CH₂O; (xxi) CH₂N(H); (xxii) CH₂S, CH₂S(O), and CH₂S(O)₂; (xxiii) CH₂S; (xxiv) CH₂S(O); (xxv) CH₂S(O)₂; (xxvi) each of (i)-(xxv) unsubstituted; (xxvii) each of (i)-(xxv) substituted with 1 or 2 substituents R^{X}; and (xxviii) each of (i)-(xxv) and (xxvii) substituted with 1 or 2 substituents R^{X} wherein R^{X} is independently 2F, CH₃, or ═O.

Another aspect of this invention is any compound of Formula I, or a pharmaceutically acceptable salt thereof, wherein R² is selected from the group consisting of any one of groups (i)-(xvii): (i) —CO₂H; (ii) —(CH₂)_{0 or 1}—N(H)-G-R; (iii) —C(O)N(H)-G-R; (iv) -G-N(H)—C(O)—R; (v) SO₃H and PO₃H₂; (vi) SO₃H; (vii) PO₃H₂; 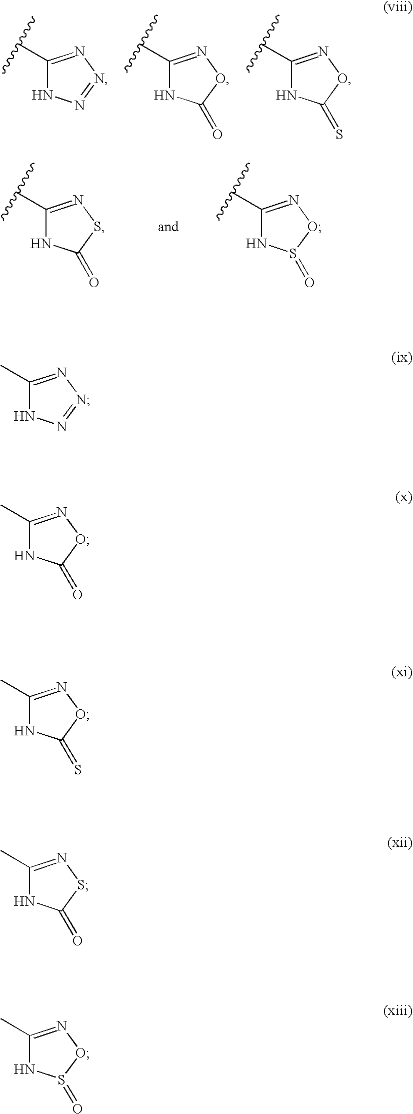
(xiv) each of (iii)-(v) wherein G is C(O); (xv) each of (iii)-(v) wherein G is S(O)₂; (xvi) each of (iii)-(v), (xv), and (xvi) wherein R is CF₃; and (xvii) each of (iii)-(v), (xv), and (xvi) wherein R is CH₃.

Another aspect of this invention is any compound of Formula I, or a pharmaceutically acceptable salt thereof, wherein V, L³, and R² are taken together to form a heterocycle radical selected from the group consisting of any one of groups (i)-(x): 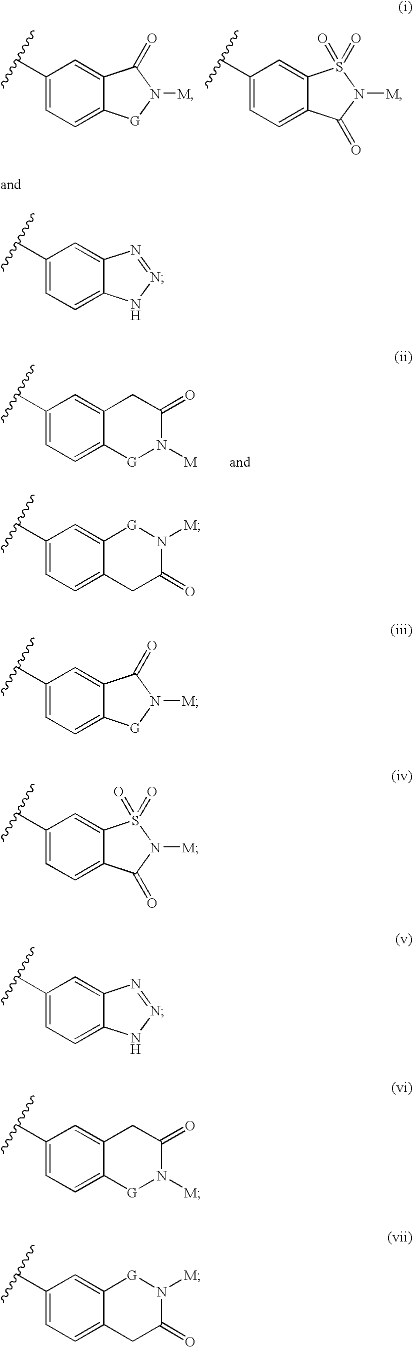 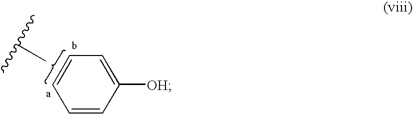
(ix) each of (i)-(iv), (vi), and (vii) wherein M is H; and (x) each of (i)-(iv), (vi), and (vii) wherein M is OH, wherein said heterocycle radical is unsubstituted or substituted with from 1 to 3 groups R^{X}, wherein R^{X} is as defined above.

Another aspect of this invention is a compound of any one of Formulas I-XI, or a pharmaceutically acceptable salt thereof, wherein R^{X} is on a carbon or nitrogen atom and is independently selected from the group consisting of any one of groups (i)-(xxxvii):
(i) C₁-C₆ alkyl, 2- to 6-membered heteroalkyl, C₃-C₅ cycloalkyl, and 3- to 5-membered heterocycloalkyl; (ii) C₁-C₆ alkyl and 2- to 6-membered heteroalkyl; (iii) C₃-C₅ cycloalkyl and 3- to 5-membered heterocycloalkyl; (iv) C₁-C₆ alkyl and C₃-C₅ cycloalkyl; (v) 2- to 6-membered heteroalkyl and 3- to 5-membered heterocycloalkyl; (vi) C₁-C₆ alkyl; (vii) C₃-C₅ cycloalkyl; (viii) 2- to 6-membered heteroalkyl; (ix) 3- to 5-membered heterocycloalkyl; (x) (C₁-C₆ alkyl)-C(O) and (C₁-C₆ alkyl)-S(O)₁₋₂; (xi) H₂NS(O)₂—, (C₁-C₆ alkyl)-N(H)S(O)₂—, and (C₁-C₆ alkyl)₂-NS(O)₂—; (xii) C₁-C₆ alkyl, 2- to 6-membered heteroalkyl, C₃-C₅ cycloalkyl, or 3- to 5-membered heterocycloalkyl substituted with F, 2F, 3F, HO—, O═, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (xiii) C₁-C₆ alkyl, 2- to 6-membered heteroalkyl, C₃-C₅ cycloalkyl, or 3- to 5-membered heterocycloalkyl substituted with F, 2F, 3F, F₃C—, or F₃CO—; (xiv) C₁-C₆ alkyl, 2- to 6-membered heteroalkyl, C₃-C₅ cycloalkyl, or 3- to 5-membered heterocycloalkyl substituted with HO—, H₃CO—, or O═; (xv) C₁-C₆ alkyl, 2- to 6-membered heteroalkyl, C₃-C₅ cycloalkyl, or 3- to 5-membered heterocycloalkyl substituted with NC—, (CH₃)₂NC(O)—, CH₃C(O)N(CH₃)—, CH₃C(O), or CH₃C(O)O—; (xvi) C₁-C₆ alkyl, 2- to 6-membered heteroalkyl, C₃-C₅ cycloalkyl, or 3- to 5-membered heterocycloalkyl substituted with H₂N—, CH₃—N(H)—, or (CH₃)₂—N—; (xvii) C₁-C₆ alkyl, 2- to 6-membered heteroalkyl, C₃-C₅ cycloalkyl, or 3- to 5-membered heterocycloalkyl substituted with HO₂C—; (xviii) C₁-C₆ alkyl, 2- to 6-membered heteroalkyl, C₃-C₅ cycloalkyl, or 3- to 5-membered heterocycloalkyl substituted with H₂NC(O)—, CH₃N(H)C(O)—, or CH₃C(O)N(H)—; (xix) C₁-C₆ alkyl, 2- to 6-membered heteroalkyl, C₃-C₅ cycloalkyl, or 3- to 5-membered heterocycloalkyl substituted with CH₃S(O)₂ or CH₃S(O)—; (xx) C₁-C₆ alkyl, 2- to 6-membered heteroalkyl, C₃-C₅ cycloalkyl, or 3- to 5-membered heterocycloalkyl substituted with CH₃S(O)₂N(H)—, or CH₃S(O)₂N(CH₃)—; (xxi) phenyl; (xxii) 5-membered heteroaryl and 6-membered heteroaryl, (xxiii) 5-membered heteroaryl; (xxiv) 6-membered heteroaryl; (xxv) phenyl, 5-membered heteroaryl, and 6-membered heteroaryl, wherein said phenyl, 5-membered heteroaryl, and 6-membered heteroaryl are substituted on a carbon atom with F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—, substituted on a carbon atom contiguous to a nitrogen atom with ═O, and optionally substituted on a nitrogen atom with CH₃; (xxvi) phenyl, 5-membered heteroaryl, and 6-membered heteroaryl, wherein said phenyl, 5-membered heteroaryl, and 6-membered heteroaryl are substituted on a carbon atom with F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (xxvii) phenyl, 5-membered heteroaryl, and 6-membered heteroaryl, wherein said phenyl, 5-membered heteroaryl, and 6-membered heteroaryl are substituted on carbon atoms with any two of F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, and CH₃S(O)₂N(CH₃)—; (xxviii) 5-membered heteroaryl and 6-membered heteroaryl, wherein said 5-membered heteroaryl and 6-membered heteroaryl are substituted on a carbon atom contiguous to a nitrogen atom with ═O; (xxix) 5-membered heteroaryl and 6-membered heteroaryl, wherein said 5-membered heteroaryl and 6-membered heteroaryl are substituted on a nitrogen atom with CH₃; (xxx) wherein each substituent R^{X} on a carbon atom is independently selected from the group consisting of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C_{C}₆ alkyl)-C(O)N(H)—, HO, F, Cl, Br, I, and HO₂C; (xxxi) wherein each substituent R^{X} on a carbon atom is independently selected from the group consisting of any two of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C₆ alkyl)-C(O)N(H)—, HO, F, Cl, Br, I, and HO₂C; (xxxii) wherein each substituent R^{X} on a carbon atom is independently selected from the group consisting of F, CH₃O, CH₃S, CH₃S(O), CH₃S(O)₂, CH₃C(O), CH₃C(O)O—, CH₃C(O)N(H)—, F₃C, HO, and HO₂C; (xxxiii) wherein two substituents R^{X} on the same carbon atom may be taken together with the carbon atom to which they are both bonded to form the group C═O; (xxxiv) wherein two adjacent substituents R^{X}, bonded to contiguous carbon atoms, may be taken together to form the diradical group —O—CH₂—O—; (xxxv) each of (i)-(xxxiv) wherein there is one R^{X}; (xxxvi) each of (i)-(xxxiv) wherein there are two substituents R^{X}; and (xxxvii) each of (i)-(xxxiv) wherein there are three substituents R^{X}.

Another aspect of this invention is a compound of any one of Formulas I-XI, or a pharmaceutically acceptable salt thereof, wherein: a 5- or 6-membered heteroaryl, or a 9- or 10-membered heterobiaryl is a monoradical ring corresponding, respectively to any one of said 5-membered heteroarylene, 6-membered heteroarylene, 9-membered heterobiarylene, or 10-membered heterobiarylene diradical rings drawn above, wherein each of the two [symbol] of the diradical rings drawn above are deleted and a single “z,1 ” indicating the point of attachment for said monoradical is instead attached at any one carbon or nitrogen atom bearing a hydrogen atom by abstraction of said hydrogen atom.

Another aspect of this invention is any compound of Formula I, or a pharmaceutically acceptable salt thereof, wherein:
R¹ is selected from the group consisting of any one of groups (i)-(vi): (i) phenyl; (ii) 5-membered heteroaryl; (iii) 6-membered heteroaryl; (iv) each of (i)-(iii) unsubstituted; (v) each of (i)-(iii) optionally substituted with from 1 to 3 substituents R^{X}; and (vi) each of (i)-(iii) and (v) optionally substituted with from 1 to 4 substituents R^{X} wherein R^{X} is independently CH₃O, CH₃S, F, Cl, CF₃, or CH₃ in the meta or para position relative to the attachment of R¹ to L¹;
L¹ is selected from the group consisting of any one of groups (i)-(vii): (i) CH₂; (ii) CH₂CH₂; (iii) S(O)₂; (iv) S(O)₂CH₂; (v) each of (i)-(iv) unsubstituted; (vi) each of (i)-(iv) substituted with 1 or 2 substituents R^{X}; and (vii) each of (i)-(iv) and (vi) substituted with 1 or 2 substituents R^{X} wherein R^{X} is independently 2F, CH₃, or ═O;
L² is selected from the group consisting of any one of groups (i)-(xii): (i) CH₂; (ii) CH₂CH₂; (iii) CH₂O; (iv) CH₂N(H); (v) S(O)CH₂; (vi) S(O)₂CH₂; (vii) CH₂S; (viii) CH₂S(O); (ix) CH₂S(O)₂; (x) each of (i)-(ix) unsubstituted; (xi) each of (i)-(ix) substituted with 1 or 2 substituents R^{X}; and (xii) each of (i)-(ix) and (xi) substituted with 1 or 2 substituents R^{X} wherein R^{X} is independently 2F, CH₃, or ═O;
V is selected from the group consisting of any one of groups (i)-(viii): (i) phenylene; (ii) 6-membered heteroarylene; (iii) C₆ cycloalkylene; (iv) 6-membered heterocycloalkylene; (v) 5-membered heteroarylene; (vi) each of (i)-(v) unsubstituted; (vii) each of (i)-(v) substituted with from 1 to 4 substituents R^{X}; and (viii) each of (i)-(v) and (vii) substituted with from 1 to 3 substituents R^{X} wherein R^{X} is F or 2F;
L³ is selected from the group consisting of any one of groups (i)-(vi): (i) CH₂; (ii) absent; (iii) CH₂CH₂; (iv) each of (i) and (iii) unsubstituted; (v) each of (i) and (iii) substituted with 1 or 2 substituents R^{X}; and (vi) each of (i) and (iii) and (v) substituted with 1 or 2 substituents R^{X} wherein R^{X} is independently 2F, CH₃, or ═O; or wherein R² is not —CO₂H, L³ is selected from the group consisting of any one of groups (i)-(xiv): (i) O; (ii) N(H); (iii) S; (iv) S(O); (v) S(O)₂; (vi) CH₂O; (vii) CH₂N(H); (viii) CH₂S; (ix) CH₂S(O); (x) CH₂S(O)₂; (xi) a bond; (xii) each of (i)-(x) unsubstituted; (xiii) each of (i)-(x) substituted with 1 or 2 substituents R^{X}; and (xiv) each of (i)-(x) and (xiii) substituted with 1 or 2 substituents R^{X} wherein R^{X} is independently 2F, CH₃, or ═O;
R² is selected from the group consisting of any one of groups (i)-(vi): (i) —CO₂H; (ii) SO₃H; (iii) PO₃H₂; (iv) —(CH₂)_{0 or 1}—N(H)-G-R, wherein G is SO₂; 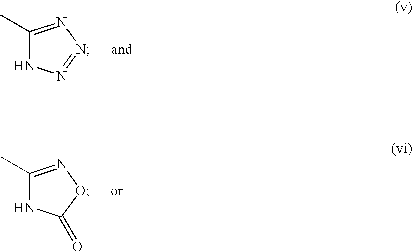
V, L³, and R² are taken together to form a heterocycle radical selected from the group consisting of any one of groups (i)-(vi): 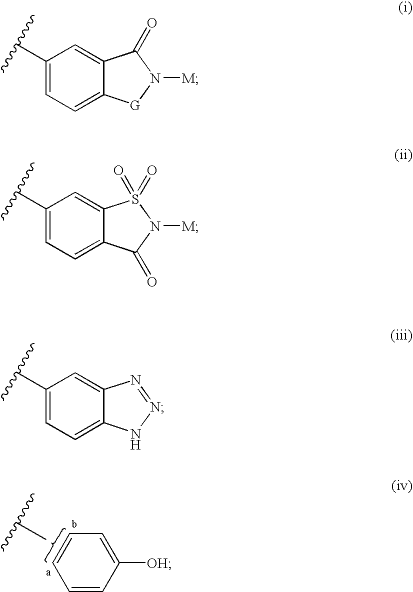
(v) each of (i)-(ii) wherein M is H; and (vi) each of (i)-(ii) wherein M is OH; wherein said heterocycle radical is unsubstituted or substituted with from 1 to 3 groups R^{X}; and
R^{X} is on a carbon or nitrogen atom and is independently selected from the
group consisting of any one of groups (i)-(xxx):
   (i) C₁-C₆ alkyl; (ii) C₃-C₅ cycloalkyl; (iii) 2- to 6-membered heteroalkyl; (iv) 3- to 5-membered heterocycloalkyl; (v) (C₁-C₆ alkyl)-C(O) and (C₁-C₆ alkyl)-S(O)₁₋₂; (vi) H₂NS(O)₂—, (C₁-C₆ alkyl)-N(H)S(O)₂—, and (C₁-C₆ alkyl)₂-NS(O)₂—; (vii) C₁-C₆ alkyl, 2- to 6-membered heteroalkyl, C₃-C₅ cycloalkyl, or 3- to 5-membered heterocycloalkyl substituted with F, 2F, 3F, HO—, O═, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (viii) C₁-C₆ alkyl, 2- to 6-membered heteroalkyl, C₃-C₅ cycloalkyl, or 3- to 5-membered heterocycloalkyl substituted with F, 2F, 3F, F₃C—, or F₃CO—; (ix) C₁-C₆ alkyl, 2- to 6-membered heteroalkyl, C₃-C₅ cycloalkyl, or 3- to 5-membered heterocycloalkyl substituted with HO—, H₃CO—, or O═; (x) C₁-C₆ alkyl, 2- to 6-membered heteroalkyl, C₃-C₅ cycloalkyl, or 3- to 5-membered heterocycloalkyl substituted with NC—, (CH₃)₂NC(O)—, CH₃C(O)N(CH₃)—, CH₃C(O), or CH₃C(O)O—; (xi) C₁-C₆ alkyl, 2- to 6-membered heteroalkyl, C₃-C₅ cycloalkyl, or 3- to 5-membered heterocycloalkyl substituted with H₂N—, CH₃—N(H)—, or (CH₃)₂—N—; (xii) C₁-C₆ alkyl, 2- to 6-membered heteroalkyl, C₃-C₅ cycloalkyl, or 3- to 5-membered heterocycloalkyl substituted with HO₂C—; (xiii) C₁-C₆ alkyl, 2- to 6-membered heteroalkyl, C₃-C₅ cycloalkyl, or 3- to 5-membered heterocycloalkyl substituted with H₂NC(O)—, CH₃N(H)C(O)—, or CH₃C(O)N(H)—; (xiv) C₁-C₆ alkyl, 2- to 6-membered heteroalkyl, C₃-C₅ cycloalkyl, or 3- to 5-membered heterocycloalkyl substituted with CH₃S(O)₂ or CH₃S(O)—; (xv) C₁-C₆ alkyl, 2- to 6-membered heteroalkyl, C₃-C₅ cycloalkyl, or 3- to 5-membered heterocycloalkyl substituted with CH₃S(O)₂N(H)—, or CH₃S(O)₂N(CH₃)—; (xvi) phenyl; (xvii) 5-membered heteroaryl; (xviii) 6-membered heteroaryl; (xix) phenyl, 5-membered heteroaryl, and 6-membered heteroaryl, wherein said phenyl, 5-membered heteroaryl, and 6-membered heteroaryl are substituted on a carbon atom with F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (xx) phenyl, 5-membered heteroaryl, and 6-membered heteroaryl, wherein said phenyl, 5-membered heteroaryl, and 6-membered heteroaryl are substituted on carbon atoms with any two of F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, and CH₃S(O)₂N(CH₃)—; (xxi) 5-membered heteroaryl and 6-membered heteroaryl, wherein said 5-membered heteroaryl and 6-membered heteroaryl are substituted on a carbon atom contiguous to a nitrogen atom with ═O; (xxii) 5-membered heteroaryl and 6-membered heteroaryl, wherein said 5-membered heteroaryl and 6-membered heteroaryl are substituted on a nitrogen atom with CH₃; (xxiii) wherein each substituent R^{X} on a carbon atom is independently selected from the group consisting of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C₆ alkyl)-C(O)N(H)—, HO, F, Cl, Br, I, and HO₂C; (xxiv) wherein each substituent R^{X} on a carbon atom is independently selected from the group consisting of any two of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C₆ alkyl)-C(O)N(H)—, HO, F, Cl, Br, I, and HO₂C; (xxv) wherein each substituent R^{X} on a carbon atom is independently selected from the group consisting of F, CH₃O, CH₃S, CH₃S(O), CH₃S(O)₂, CH₃C(O), CH₃C(O)O—, CH₃C(O)N(H)—, F₃C, HO, and HO₂C; (xxvi) wherein two substituents R^{X} on the same carbon atom may be taken together with the carbon atom to which they are both bonded to form the group C═O; (xxvii) wherein two adjacent substituents R^{X}, bonded to contiguous carbon atoms, may be taken together to form the diradical group —O—CH₂—O—; (xxviii) each of (i)-(xxvii) wherein there is one R^{X}; (xxix) each of (i)-(xxvii) wherein there are two substituents R^{X}; and (xxx) each of (i)-(xxvii) wherein there are three substituents R^{X}.

Another aspect of this invention is any compound of Formula I, or a pharmaceutically acceptable salt thereof, wherein
R¹ is selected from the group consisting of any one of groups (i)-(v): (i) phenyl; (ii) 5-membered heteroaryl; (iii) 6-membered heteroaryl; (iv) each of (i)-(iii) unsubstituted; and (v) each of (i)-(iii) optionally substituted with from 1 to 3 substituents R^{X} wherein R^{X} is in the meta or para position relative to the attachment of R¹ to L¹;
L¹ and L² are independently selected from the group consisting of any one of groups (i)-(iv): (i) CH₂; (ii) each of (i) unsubstituted; (iii) each of (i) substituted with 1 or 2 substituents R^{X}; and (iv) each of (i) substituted with 1 or 2 substituents R^{X} wherein R^{X} is independently 2F, CH₃, or ═O;
V is selected from the group consisting of any one of groups (i)-(vii): (i) phenylene; (ii) 6-membered heteroarylene; (iii) C₆ cycloalkylene; (iv) 6-membered heterocycloalkylene; (v) each of (i)-(iv) unsubstituted; (vi) each of (i)-(iv) substituted with from 1 to 3 substituents R^{X}; and (vii) each of (i)-(iv) and (vi) substituted with from 1 to 3 substituents R^{X} wherein R^{X} is F or 2F;
L³ is selected from the group consisting of any one of groups (i)-(v): (i) CH₂; (ii) absent; (iii) each of (i) unsubstituted; (iv) each of (i) substituted with 1 or 2 substituents R^{X}; and (v) each of (i) and (iv) substituted with 1 or 2 substituents R^{X}, wherein R^{X} is independently 2F, CH₃, or ═O; or wherein R² is not CO₂H, L³ is selected from the group consisting of any one of groups (i)-(ix): (i) O; (ii) N(H); (iii) S; (iv) S(O); (v) S(O)₂; (vi) a bond; (vii) each of (i)-(v) unsubstituted; (viii) each of (i)-(v) substituted with 1 or 2 substituents R^{X}; and (ix) each of (i)-(x) and (xii) substituted with 1 or 2 substituents R^{X} wherein R^{X} is independently 2F, CH₃, or ═O;
R² is selected from the group consisting of any one of groups (i)-(iv): (i) —CO₂H; (ii) —(CH₂)_{0 or 1}—N(H)-G-R, wherein G is SO₂; 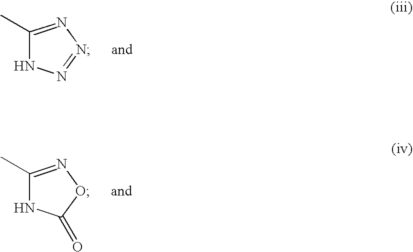
R^{X} is on a carbon or nitrogen atom and is independently selected from the group consisting of any one of groups (i) - (xxx):
   (i) C₁-C₆ alkyl; (ii) C₃-C₅ cycloalkyl; (iii) 2- to 6-membered heteroalkyl; (iv) 3- to 5-membered heterocycloalkyl; (v) (C₁-C₆ alkyl)-C(O) and (C₁-C₆ alkyl)-S(O)₁₋₂; (vi) H₂NS(O)₂—, (C₁-C₆ alkyl)-N(H)S(O)₂—, and (C₁-C₆ alkyl)₂-NS(O)₂—; (vii) C₁-C₆ alkyl, 2- to 6-membered heteroalkyl, C₃-C₅ cycloalkyl, or 3- to 5-membered heterocycloalkyl substituted with F, 2F, 3F, HO—, O═, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (viii) C₁-C₆ alkyl, 2- to 6-membered heteroalkyl, C₃-C₅ cycloalkyl, or 3- to 5-membered heterocycloalkyl substituted with F, 2F, 3F, F₃C—, or F₃CO—; (ix) C₁-C₆ alkyl, 2- to 6-membered heteroalkyl, C₃-C₅ cycloalkyl, or 3- to 5-membered heterocycloalkyl substituted with HO—, H₃CO—, or O═; (x) C₁-C₆ alkyl, 2- to 6-membered heteroalkyl, C₃-C₅ cycloalkyl, or 3- to 5-membered heterocycloalkyl substituted with NC—, (CH₃)₂NC(O)—, CH₃C(O)N(CH₃)—, CH₃C(O), or CH₃C(O)O—; (xi) C₁-C₆ alkyl, 2- to 6-membered heteroalkyl, C₃-C₅ cycloalkyl, or 3- to 5-membered heterocycloalkyl substituted with H₂N—, CH₃—N(H)—, or (CH₃)₂—N—; (xii) C₁-C₆ alkyl, 2- to 6-membered heteroalkyl, C₃-C₅ cycloalkyl, or 3- to 5-membered heterocycloalkyl substituted with HO₂C—; (xiii) C₁-C₆ alkyl, 2- to 6-membered heteroalkyl, C₃-C₅ cycloalkyl, or 3- to 5-membered heterocycloalkyl substituted with H₂NC(O)—, CH₃N(H)C(O)—, or CH₃C(O)N(H)—; (xiv) C₁-C₆ alkyl, 2- to 6-membered heteroalkyl, C₃-C₅ cycloalkyl, or 3- to 5-membered heterocycloalkyl substituted with CH₃S(O)₂ or CH₃S(O)—; (xv) C₁-C₆ alkyl, 2- to 6-membered heteroalkyl, C₃-C₅ cycloalkyl, or 3- to 5-membered heterocycloalkyl substituted with CH₃S(O)₂N(H)—, or CH₃S(O)₂N(CH₃)—; (xvi) phenyl; (xvii) 5-membered heteroaryl; (xviii) 6-membered heteroaryl; (xix) phenyl, 5-membered heteroaryl, and 6-membered heteroaryl, wherein said phenyl, 5-membered heteroaryl, and 6-membered heteroaryl are substituted on a carbon atom with F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (xx) phenyl, 5-membered heteroaryl, and 6-membered heteroaryl, wherein said phenyl, 5-membered heteroaryl, and 6-membered heteroaryl are substituted on carbon atoms with any two of F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, and CH₃S(O)₂N(CH₃)—; (xxi) 5-membered heteroaryl and 6-membered heteroaryl, wherein said 5-membered heteroaryl and 6-membered heteroaryl are substituted on a carbon atom contiguous to a nitrogen atom with ═O; (xxii) 5-membered heteroaryl and 6-membered heteroaryl, wherein said 5-membered heteroaryl and 6-membered heteroaryl are substituted on a nitrogen atom with CH₃; (xxiii) wherein each substituent R^{X} on a carbon atom is independently selected from the group consisting of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O)O—, (C₁-C₆ alkyl)-C(O)N(H)—, HO, F, Cl, Br, I, and HO₂C; (xxiv) wherein each substituent R^{X} on a carbon atom is independently selected from the group consisting of any two of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C₆ alkyl)-C(O)N(H)—, HO, F, Cl, Br, I, and HO₂C; (xxv) wherein each substituent R^{X} on a carbon atom is independently selected from the group consisting of F, CH₃O, CH₃S, CH₃S(O), CH₃S(O)₂, CH₃C(O), CH₃C(O)O—, CH₃C(O)N(H)—, F₃C, HO, and HO₂C; (xxvi) wherein two substituents R^{X} on the same carbon atom may be taken together with the carbon atom to which they are both bonded to form the group C═O; (xxvii) wherein two adjacent substituents R^{X}, bonded to contiguous carbon atoms, may be taken together to form the diradical group —O—CH₂—O—; (xxviii) each of (i)-(xxvii) wherein there is one R^{X}; (xxix) each of (i)-(xxvii) wherein there are two substituents R^{X}; and (xxx) each of (i)-(xxvii) wherein there are three substituents R^{X}.

Another aspect of this invention is any compound of Formula I, or a pharmaceutically acceptable salt thereof, wherein
R¹ is selected from the group consisting of any one of groups (i)-(v): (i) phenyl; (ii) 5-membered heteroaryl; (iii) 6-membered heteroaryl; (iv) each of (i)-(iii) unsubstituted; and (v) each of (i)-(iii) optionally substituted with from 1 to 3 substituents R^{X} wherein R^{X} is in the meta or para position relative to the attachment of R¹ to L¹;
L¹ and L² are independently selected from the group consisting of any one of groups (i)-(iv): (i) CH₂; (ii) each of (i) unsubstituted; (iii) each of (i) substituted with 1 or 2 substituents R^{X}; and (iv) each of (i) substituted with 1 or 2 substituents R^{X} wherein R^{X} is independently 2F, CH₃, or ═O;
V is selected from the group consisting of any one of groups (i)-(vii): (i) phenylene wherein the radicals are para to each other; (ii) 6-membered heteroarylene wherein the radicals are para to each other; (iii) C₆ cycloalkylene wherein the radicals are para to each other; (iv) 6-membered heterocycloalkylene wherein the radicals are para to each other; (v) each of (i)-(iv) unsubstituted; (vi) each of (i)-(iv) substituted with from 1 to 3 substituents R^{X}; and (vii) each of (i)-(iv) and (vi) substituted with from 1 to 3 substituents R^{X} wherein R^{X} is F or 2F;
L³ is selected from the group consisting of any one of groups (i)-(v): (i) CH₂; (ii) absent; (iii) each of (i) unsubstituted; (iv) each of (i) substituted with 1 or 2 substituents R^{X}; and (v) each of (i) and (iv) substituted with 1 or 2 substituents R^{X} wherein R^{X} is independently 2F, CH₃, or ═O;
R² is selected from the group consisting of any one of groups (i)-(iv): (i) —CO₂H; (ii) —(CH₂)_{0 or 1}—N(H)-G-R, wherein G is SO₂; 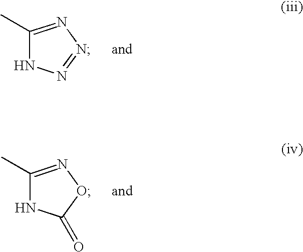
R^{X} is on a carbon or nitrogen atom and is independently selected from the group consisting of any one of groups (i) - (xxvi):
   (i) C₁-C₆ alkyl; (ii) C₃-C₅ cycloalkyl; (iii) 2- to 6-membered heteroalkyl; (iv) 3- to 5-membered heterocycloalkyl; (v) C₁-C₆ alkyl, 2- to 6-membered heteroalkyl, C₃-C₅ cycloalkyl, or 3- to 5-membered heterocycloalkyl substituted with F, 2F, 3F, F₃C—, or F₃CO—; (vi) C₁-C₆ alkyl, 2- to 6-membered heteroalkyl, C₃-C₅ cycloalkyl, or 3- to 5-membered heterocycloalkyl substituted with HO—, H₃CO—, or O═; (vii) C₁-C₆ alkyl, 2- to 6-membered heteroalkyl, C₃-C₅ cycloalkyl, or 3- to 5-membered heterocycloalkyl substituted with NC—, (CH₃)₂NC(O)—, CH₃C(O)N(CH₃)—, CH₃C(O), or CH₃C(O)O—; (viii) C₁-C₆ alky, 2- to 6-membered heteroalkyl, C₃-C₅ cycloalkyl, or 3- to 5-membered heterocycloalkyl substituted with H₂N—, CH₃—N(H)—, or (CH₃)₂—N—; (ix) C₁-C₆ alkyl, 2- to 6-membered heteroalkyl, C₃-C₅ cycloalkyl, or 3- to 5-membered heterocycloalkyl substituted with HO₂C—; (x) C₁-C₆ alkyl, 2- to 6-membered heteroalkyl, C₃-C₅ cycloalkyl, or 3- to 5-membered heterocycloalkyl substituted with H₂NC(O)—, CH₃N(H)C(O)—, or CH₃C(O)N(H)—; (xi) C₁-C₆ alkyl, 2- to 6-membered heteroalkyl, C₃-C₅ cycloalkyl, or 3- to 5-membered heterocycloalkyl substituted with CH₃S(O)₂ or CH₃S(O)—; (xii) C₁-C₆ alkyl, 2- to 6-membered heteroalkyl, C₃-C₅ cycloalkyl, or 3- to 5-membered heterocycloalkyl substituted with CH₃S(O)₂N(H)—, or CH₃S(O)₂N(CH₃)—; (xiii) phenyl; (xiv) 5-membered heteroaryl; (xv) 6-membered heteroaryl; (xvi) phenyl, 5-membered heteroaryl, and 6-membered heteroaryl, wherein said phenyl, 5-membered heteroaryl, and 6-membered heteroaryl are substituted on a carbon atom with F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, or CH₃S(O)₂N(CH₃)—; (xvii) phenyl, 5-membered heteroaryl, and 6-membered heteroaryl, wherein said phenyl, 5-membered heteroaryl, and 6-membered heteroaryl are substituted on carbon atoms with any two of F, HO—, F₃C—, H₃CO—, F₃CO—, NC—, H₂N—, CH₃—N(H)—, (CH₃)₂—N—, HO₂C—, H₂NC(O)—, CH₃N(H)C(O)—, (CH₃)₂NC(O)—, CH₃C(O)N(H)—, CH₃C(O)N(CH₃)—, CH₃C(O), CH₃C(O)O—, CH₃S(O)₂, CH₃S(O)—, CH₃S(O)₂N(H)—, and CH₃S(O)₂N(CH₃)—; (xviii) 5-membered heteroaryl and 6-membered heteroaryl, wherein said 5-membered heteroaryl and 6-membered heteroaryl are substituted on a carbon atom contiguous to a nitrogen atom with ═O; (xix) wherein each substituent R^{X} on a carbon atom is independently selected from the group consisting of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (C₁-C₆ alkyl)-C(O)O—, (C₁-C₆ alkyl)-C(O)N(H)—, HO, F, Cl, Br, I, and HO₂C; (xx) wherein each substituent R^{X} on a carbon atom is independently selected from the group consisting of any two of: (C₁-C₆ alkyl)-O, (C₁-C₆ alkyl)-S, H₂N, (C₁-C₆ alkyl)-N(H)—, (C₁-C₆ alkyl)₂-N—, (C₁-C₆ alkyl)-C(O), (Cl-C₆ alkyl)-C(O)O—, (C₁-C₆ alkyl)-C(O)N(H)—, HO, F, Cl, Br, I, and HO₂C; (xxi) wherein each substituent R^{X} on a carbon atom is independently selected from the group consisting of F, CH₃O, CH₃S, CH₃S(O), CH₃S(O)₂, CH₃C(O), CH₃C(O)O—, CH₃C(O)N(H)—, F₃C, HO, and HO₂C; (xxii) wherein two substituents R^{X} on the same carbon atom may be taken together with the carbon atom to which they are both bonded to form the group C═O; (xxiii) wherein two adjacent substituents R^{X}, bonded to contiguous carbon atoms, may be taken together to form the diradical group —O—CH₂—O—; (xxiv) each of (i)-(xxii) wherein there is one R^{X}; (xxv) each of (i)-(xxii) wherein there are two substituents R^{X}; and (xxvi) each of (i)-(xxii) wherein there are three substituents R^{X}.

Another aspect of this invention is a compound of Formula I, or a pharmaceutically acceptable salt thereof, wherein
R¹ is phenyl or pyridyl, wherein phenyl and pyridyl are unsubstituted or substituted by F, Cl, 2F, (C₁-C₆ alkyl)-O, substituted C₁-C₆ alkyl, F and (C₁-C₆ alkyl)-O, (C₁-C₆ alkyl)-S, or (C₁-C₆ alkyl)-S(O)₁₋₂;
L¹ is CH₂;
L² is CH₂;
V is phenylene or C₆ cycloalkyl, wherein the radicals are 1,4 to each other;
L³ is absent; and
R² is CO₂H, CH₂N(H)S(O)₂—(C₁-C₆ alkyl), or tetrazol-5-yl.

Another aspect of this invention is a compound of Formula I, or a pharmaceutically acceptable salt thereof, wherein
R¹ is phenyl or pyridyl, wherein phenyl and pyridyl are unsubstituted or substituted by F, Cl, 2F, CH₃O, CF₃, F and CH₃O, CH₃S, CH₃S(O), or CH₃S(O)₂;
L¹ is CH₂;
L² is CH₂;
V is phenylene or C₆ cycloalkyl, wherein the radicals are 1,4 to each other;
L³ is absent; and
R² is CO₂H, CH₂N(H)S(O)₂—CH₃, or tetrazol-5-yl.

Another aspect of this invention is a compound of Formula I, or a pharmaceutically acceptable salt thereof, wherein
R¹ is phenyl or pyridyl, wherein phenyl and pyridyl are unsubstituted or substituted by F, Cl, 2F, (C₁-C₆ alkyl)-O, (C₁-C₆ alkyl)-S, (C₁-C₆ alkyl)-S(O)₁₋₂, substituted C₁-C₆ alkyl, or F and CH₃O;
L¹ is CH₂;
L² is CH₂;
V is phenylene or C₆ cycloalkyl, wherein the radicals are 1,4 to each other and further are oriented trans to each other on C₆ cycloalkyl;
L³ is absent; and
R² is CO₂H or tetrazol-5-yl.

Another aspect of this invention is a compound of Formula I, or a pharmaceutically acceptable salt thereof, wherein
R¹ is phenyl or pyridyl, wherein phenyl and pyridyl are unsubstituted or substituted by F, Cl, 2F, (C₁-C₆ alkyl)-O, (C₁-C₆ alkyl)-S, (C₁-C₆ alkyl)-S(O)₁₋₂, CF₃, or F and CH₃O;
L¹ is CH₂;
L² is CH₂;
V is phenylene or C₆ cycloalkyl, wherein the radicals are 1,4 to each other and further are oriented cis to each other on C₆ cycloalkyl;
L³ is absent; and
R² is CO₂H or tetrazol-5-yl.

Another aspect of this invention is a compound of Formula I, or a pharmaceutically acceptable salt thereof, wherein
R¹ is phenyl or pyridyl, wherein phenyl and pyridyl are unsubstituted or substituted by F, Cl, 2F, (C₁-C₆ alkyl)-O, (C₁-C₆ alkyl)-S, (C₁-C₆ alkyl)-S(O)₁₋₂, CF₃, or F and CH₃O;
L¹ is CH₂;
L² is CH₂;
V is phenylene or C₆ cycloalkyl, wherein the radicals are 1,4 to each other and further are oriented trans to each other on C₆ cycloalkyl;
L³is absent; and
R² is CO₂H.

Another aspect of this invention is a compound of Formula I, or a pharmaceutically acceptable salt thereof, selected from the group consisting of a compound of any one of the Compound Examples described below, or a pharmaceutically acceptable salt thereof, or crystal form thereof.

Another aspect of this invention is a compound selected from the group consisting of:
4-[6-(3-fluoro-4-methoxy-benzyl-carbamoyl)-4-oxo-4H-pyrido-[3,4-d]pyrimidin-3-ylmethyl]-benzoic acid;
3-[4-(methane-sulfonylamino-methyl)-benzyl]-4-oxo-3,4-dihydro-pyrido[3,4-d]-pyrimidin-6-caboxylic acid 4-methoxy-benzyl-amide;
3-(cyano-benzyl)-4-oxo-3,4-dihydro-pyrido[3,4-d]pyrimidine-6-carboxylic acid 4-methoxy-benzyl-amide;
4-oxo-3-[4-(2H)-tetrazol-5-yl)-benzyl]-3,4-dihydro-pyrido-[3,4-d]pyrimidine-6-carboxylic acid 4-methoxy-benzyl-amide;
4-oxo-3-[4-(2H)-tetrazol-5-yl)-benzyl]-3,4-dihydro-pyrido-[3,4-d]pyrimidine-6-carboxylic acid 3-methoxy-benzyl-amide; or
a pharmaceutically acceptable salt thereof.

Another aspect of this invention is a compound selected from the group consisting of:
4-[6-(4-methoxy-benzylcarbamoyl)-4-oxo-4H-pyrido[3,4-d]pyrimidin-3-ylmethyl]-benzoic acid glucosamine salt.

Another aspect of this invention is a compound selected from the group consisting of:
4-[6-(4-methoxy-benzylcarbamoyl)-4-oxo-4H-pyrido[3,4-d]pyrimidin-3-ylmethyl]-benzoic acid ⅓ H₃PO₄;
4-[6-(4-methoxy-benzylcarbamoyl)-4-oxo-4H-pyrido[3,4-d]pyrimidin-3-ylmethyl]-benzoic acid monohydrochloride;
4-[6-(4-methoxy-benzylcarbamoyl)-4-oxo-4H-pyrido[3,4-d]pyrimidin-3-ylmethyl]-benzoic acid mono hydrobromide;
4-[6-(4-methoxy-benzylcarbamoyl)-4-oxo-4H-pyrido[3,4-d]pyrimidin-3-ylmethyl]-benzoic acid ½ H₂SO₄;
4-[6-(4-methoxy-benzylcarbamoyl)-4-oxo-4H-pyrido[3,4-d]pyrimidin-3-ylmethyl]-benzoic acid mesylate;
4-[6-(4-methoxy-benzylcarbamoyl)-4-oxo-4H-pyrido[3,4-d]pyrimidin-3-ylmethyl]-benzoic acid besylate;
4-[6-(4-methoxy-benzylcarbamoyl)-4-oxo-4H-pyrido[3,4-d]pyrimidin-3-ylmethyl]-benzoic acid camsylate; and
4-[6-(4-methoxy-benzylcarbamoyl)-4-oxo-4H-pyrido[3,4-d]pyrimidin-3-ylmethyl]-benzoic acid edisylate.

Another aspect of this invention is a compound selected from the group consisting of:
4-Oxo-3-[1-(5-oxo-4,5-dihydro-[1,2,4]oxadiazol-3-ylmethyl)-1H-pyrrol-3-yloxymethyl]-3,4-dihydro-pyrido[3,4-d]pyrimidine-6-carboxylic acid (pyridin-3-ylmethyl)-amide, which has the structure: 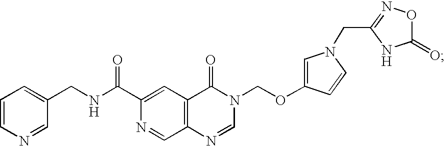
3-[2-(4-Methanesulfonylaminocarbonyl-benzo[b]thiophen-7-yl)-ethyl]-4-oxo-3,4-dihydro-pyrido[3,4-d]pyrimidine-6-carboxylic acid (1H-benzoimidazol-5-yloxymethyl)-amide, which has the structure: 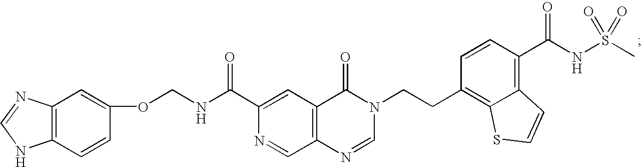
4-Oxo-3-{[3-(5-thioxo-4,5-dihydro-[1,2,4]oxadiazol-3-ylmethoxy)-cyclopentylamino]-methyl}-3,4-dihydro-pyrido[3,4-d]pyrimidine-6-carboxylic acid (naphthalen-2-ylaminomethyl)-amide, which has the structure: 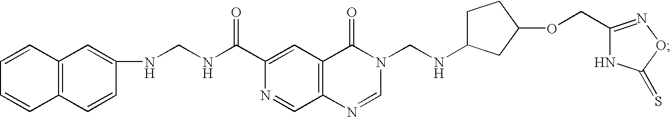
4-Oxo-3-[1-(2,2,2-trifluoro-acetylsulfamoyl)-piperidin-4-ylmethanesulfonyl]-3,4-dihydro-pyrido[3,4-d]pyrimidine-6-carboxylic acid (isothiazole-3-sulfinylmethyl)-amide, which has the structure: 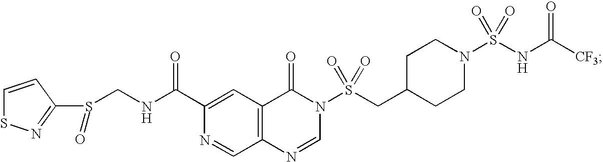
Phosphoric acid mono-{4-[4-oxo-6-(2-quinolin-3-yl-ethylcarbamoyl)-4H-pyrido[3,4-d]pyrimidine-3-sulfonyl]-adamantan-1-yl}ester, which has the structure: 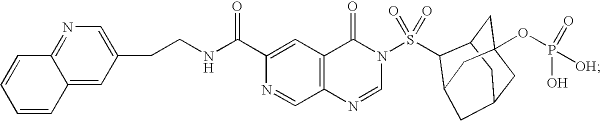
1-[4-Oxo-6-(phenylsulfanylmethyl-carbamoyl)-4H-pyrido[3,4-d]pyrimidin-3-ylmethanesulfonyl]-octahydro-indole-3-sulfonic acid, which has the structure: 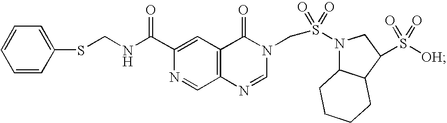
4-Oxo-3-[6-(5-oxo-4,5-dihydro-[1,2,4]thiadiazol-3-yl)-naphthalen-2-yloxymethyl]-3,4-dihydro-pyrido[3,4-d]pyrimidine-6-carboxylic acid (indole-1-sulfonylmethyl)-amide, which has the structure: 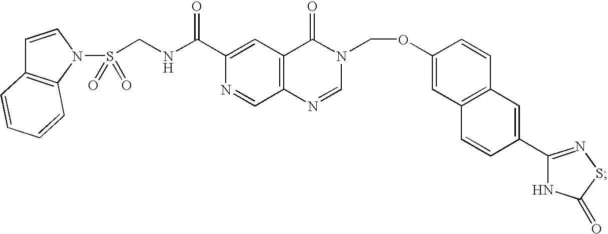
4-Oxo-3-[5-(2-oxo-2,3-dihydro-214-[1,2,3,5]oxathiadiazol-4-yl)-pyridin-3-ylmethyl]-3,4-dihydro-pyrido[3,4-d]pyrimidine-6-carboxylic acid (isoxazole-5-sulfinylmethyl)-amide, which has the structure: 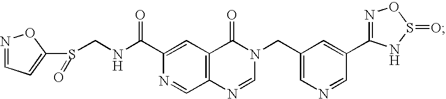
N-[3-(4-Hydroxy-phenylmethanesulfinyl)-4-oxo-3,4-dihydro-pyrido[3,4-d]pyrimidine-6-carbonyl]-C-phenyl-methanesulfonamide, which has the structure: 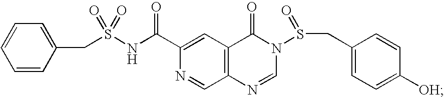
3-(2-Hydroxy-1,1,3-trioxo-2,3-dihydro-1H-116-benzo[d]isothiazol-5-ylmethyl)-4-oxo-3,4-dihydro-pyrido[3,4-d]pyrimidine-6-carboxylic acid (pyridazin-3-ylmethyl)-amide, which has the structure: 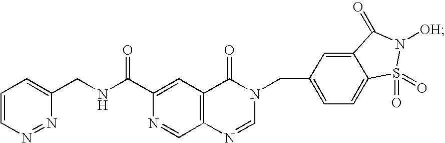
4-Oxo-3-(1,1,3-trioxo-2,3-dihydro-1H-116-benzo[d]isothiazol-6-ylmethyl)-3,4-dihydro-pyrido[3,4-d]pyrimidine-6-carboxylic acid (benzo[1,3]dioxol-5-ylmethyl)-amide, which has the structure: 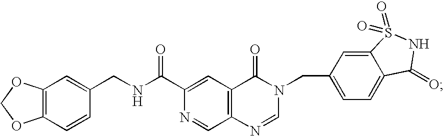
3-(1H-Benzotriazol-5-ylmethyl)-4-oxo-3,4-dihydro-pyrido[3,4-d]pyrimidine-6-carboxylic acid 4-methyl-benzylamide, which has the structure: 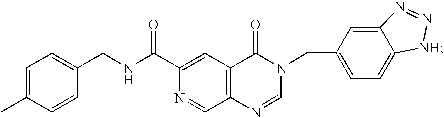
3-(2-Hydroxy-1,3-dioxo-1,2,3,4-tetrahydro-isoquinolin-6-ylmethyl)-4-oxo-3,4-dihydro-pyrido[3,4-d]pyrimidine-6-carboxylic acid (1,3-dimethyl-2,3-dihydro-1H-benzoimidazol-5-ylmethyl)-amide, which has the structure: 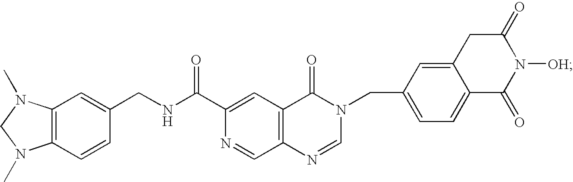
3-[Difluoro-(1,1,3-trioxo-1,2,3,4-tetrahydro-116-benzo[e][1,2]thiazin-7-yl)-methyl]-4-oxo-3,4-dihydro-pyrido[3,4-d]pyrimidine-6-carboxylic acid 4-acetyl-benzylamide, which has the structure: 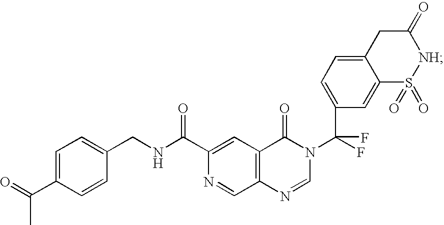
3-(2-Hydroxy-1,1,3-trioxo-2,3-dihydro-1H-116-benzo[d]isothiazole-6-carbonyl)-4-oxo-3,4-dihydro-pyrido[3,4-d]pyrimidine-6-carboxylic acid (3-oxo-3H-isoxazol-2-ylmethyl)-amide, which has the structure: 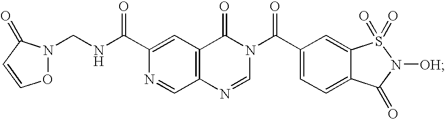
   and
Pyrazine-2-carboxylic acid [4-oxo-3-(1,1,3-trioxo-2,3-dihydro-1H-116-benzo[d]isothiazol-5-ylmethyl)-3,4-dihydro-pyrido[3,4-d]pyrimidine-6-carbonyl]-amide, which has the structure: 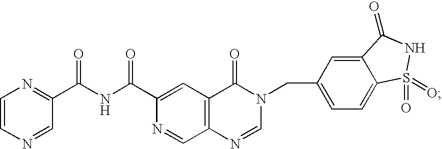
or a pharmaceutically acceptable salt thereof.

Another aspect of this invention is a crystal form of a compound of Formula I, or a pharmaceutically acceptable salt thereof. Another aspect of this invention is a crystal form of a compound of any one of Formulas II-XI, or a pharmaceutically acceptable salt thereof. In another aspect of this invention, said crystal form is Crystal Form 1 of 4-[6-(4-methoxy-benzylcarbamoyl)-4-oxo-4H-pyrido[3,4-d]pyrimidin-3-ylmethyl]-benzoic acid.

Another aspect of this invention is a combination, comprising a compound of Formula I, or a pharmaceutically acceptable salt thereof, together with another pharmaceutically active component as described herein. Another aspect of this invention is a combination, comprising any one aspect of a compound of Formula I described herein, or a pharmaceutically acceptable salt thereof, or any one aspect of a crystal form described herein, or a pharmaceutically acceptable salt thereof, together with another pharmaceutically active component as described herein. The pharmaceutically active components of said combinations may be administered together or separately. Said combinations may, or may not, be administered as part of a pharmaceutical formulation.

Another aspect of this invention is a combination, comprising any one aspect of a compound of Formula I described herein, or a pharmaceutically acceptable salt thereof, or any one aspect of a crystal form described herein, or a pharmaceutically acceptable salt thereof, together with a COX-2 inhibitor, or a pharmaceutically acceptable salt thereof, selected from the group consisting of:
ABT-963;
Valdecoxib;
BMS-347070;
Celecoxib;
Tilacoxib;

The compound of formula (B) 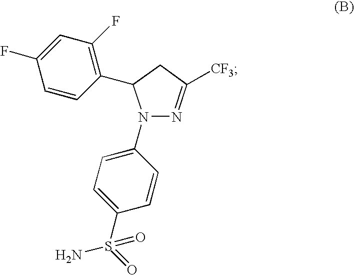
CS-502 [Chemical Abstracts Service Registry Number (“CAS Reg. No.”) 176429-82-6];
(6aR,10aR)-3-(1,1-dimethylheptyl)-6a,7,10,10a-tetrahydro-1-hydroxy-6,6-dimethyl-6H-dibenzo[b,d]pyran-9-carboxylic acid (“CT-3”);
   CV-247;
   2(5H)-Furanone, 5,5-dimethyl-3-(1-methylethoxy)-4-[4-(methylsulfonyl)phenyl]-(“DFP”);
   Etoricoxib (tradename ARCOXIA® by MERCK & CO., Inc., Whitehouse Station, N.J.);
   GW-406381;
   Tiracoxib;
   Meloxicam;
   Nimesulide;
2-(Acetyloxy)benzoic acid, 3-[(nitrooxy)methyl]phenyl ester (“NCX-4016”);
Parecoxib (trade name application pending for DYNASTAT® by G. D. Searle & Co., Skokie, Ill.);
   P54 (CAS Reg. No. 130996-28-0);
Rofecoxib (tradename VIOXX® by MERCK & CO., Inc., Whitehouse Station, N.J.);
   R [symbol] ylMiD;
2,6-Bis(1,1-dimethylethyl)-4-[(E)-(2-ethyl-1,1-dioxo-5-isothiazolidinylidene)methyl]phenol (“S-2474”);
   5(R)-Thio-6-sulfonamide-3(2H)-benzofuranone (“SVT-2016”); and
   N-[3-(Formylamino)-4-oxo-6-phenoxy-4H-1-benzopyran-7-yl]-methanesulfonamide (“T-614”); or
   a pharmaceutically acceptable salt thereof.

The invention also provides a combination, comprising any one aspect of a compound of Formula I described herein, or a pharmaceutically acceptable salt thereof, or any one aspect of a crystal form described herein, or a pharmaceutically acceptable salt thereof, together with methotrexate or leflunomide (e.g., ARAVA®).

The invention also provides a combination, comprising any one aspect of a compound of Formula I described herein, or a pharmaceutically acceptable salt thereof, or any one aspect of a crystal form described herein, or a pharmaceutically acceptable salt thereof, together with a biologic therapeutic agent selected from the group consisting of: CP-870, etanercept, infliximab, methotrexate, and adalimumab.

Still another aspect of this invention is any one of said combinations wherein the compound of Formula I, or a pharmaceutically acceptable salt thereof, is a compound of any one of Formulas II-XI, or a pharmaceutically acceptable salt thereof.

Another aspect of this invention is a pharmaceutical composition, comprising a compound of Formula I, or a pharmaceutically acceptable salt thereof, together with a pharmaceutically acceptable carrier, diluent, or excipient. In another aspect of this invention, said pharmaceutical composition comprises any one aspect of said compound of Formula I, or said pharmaceutically acceptable salt thereof, described herein.

Another aspect of this invention is a pharmaceutical composition, comprising a crystal form of a compound of Formula I, or a pharmaceutically acceptable salt thereof, together with a pharmaceutically acceptable carrier, diluent, or excipient. In another aspect of this invention, said pharmaceutical composition comprises Crystal Form 1 of 4-[6-(4-methoxy-benzylcarbamoyl)-4-oxo-4H-pyrido[3,4-d]pyrimidin-3-ylmethyl]-benzoic acid, together with a pharmaceutically acceptable carrier, diluent, or excipient.

Another aspect of this invention is said pharmaceutical composition as described below in Formulations A to E. The formulations are not to be construed as limiting the invention in any respect.

FORMULATION A

| Tablet Formulation: | |
|---|---|
| Ingredient | Amount (mg) |
| Said invention compound, or said salt thereof | 25 |
| Lactose | 50 |
| Cornstarch (for mix) | 10 |
| Cornstarch (paste) | 10 |
| Magnesium stearate (1%) | 5 |
| Total | 100 |

Said invention compound, or said salt thereof, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered from one to four times a day to a mammal, including a human, suffering from, or predicted to suffer from, a disease mediated by an MMP-13 enzyme.

### Formulation B

Injection vials:

The pH of a solution of 500 g of said invention compound, or said salt thereof, and 5 g of disodium hydrogen phosphate is adjusted to pH 6.5 in 3 L of double-distilled water using 2 M hydrochloric acid. The solution is sterile filtered, and the filtrate is filled into injection vials, lyophilized under sterile conditions, and aseptically sealed. Each injection vial contains 25 mg of said invention compound or said salt thereof.

### Formulation C

Capsules:

2 kg of said invention compound, or said salt thereof, are filled into hard gelatin capsules in a customary manner such that each capsule contains 25 mg of said invention compound, or said salt thereof.

The following Formulation D illustrates the invention pharmaceutical compositions containing an invention combination in a single formulation with a pharmaceutically acceptable carrier, diluent, or excipient.

FORMULATION D

| Tablet Formulation: | |
|---|---|
| Ingredient | Amount (mg) |
| Said invention compound, or said salt thereof | 25 |
| A COX-2 inhibitor | 20 |
| Lactose | 50 |
| Cornstarch (for mix) | 10 |
| Cornstarch (paste) | 10 |
| Magnesium stearate (1%) | 5 |
| Total | 120 |

Said invention compound, or said salt thereof, and the COX-2 inhibitor, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered from one to four times a day to a mammal, including a human, suffering from, or predicted to suffer from, a disease mediated by an MMP-13 enzyme and a disease mediated by a COX-2 enzyme.

While it may be desirable to formulate said invention compound, or said salt thereof, and another pharmaceutically active ingredient such as a COX-2 inhibitor together in one capsule, tablet, ampoule, solution, and the like, for simultaneous administration, it is not necessary for the purposes of practicing the invention methods of treating. For example, said invention compound, or said salt thereof, and said COX-2 inhibitor alternatively can each be formulated independently in any form such as, for example, those of any one Formulations A to C, and administered to a patient either simultaneously or at different times.

The following Formulation E illustrates the invention pharmaceutical compositions containing discrete formulations of the active components of an invention combination and a pharmaceutically acceptable carrier, diluent, or excipient.

### Formulation E

Capsule formulation of said invention compound, or salt thereof, is prepared according to the method of Formulation C.

Coated Tablet Formulation of a COX-2 Inhibitor:

| Ingredient | Amount (mg) |
|---|---|
| COX-2 inhibitor | 25 |
| Lactose | 50 |
| Cornstarch (for mix) | 10 |
| Cornstarch (paste) | 10 |
| Magnesium stearate (1%) | 5 |
| Total | 100 |

The COX-2 inhibitor, lactose, and cornstarch (for mix) are blended to uniformity. The cornstarch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. The resulting tablets are coated in a customary manner with a coating of sucrose, potato starch, talc, tragacanth, and colorant.

Such coated tablets containing the COX-2 inhibitor can be orally administered one or two times a day to a mammal, including a human, suffering from a disease mediated by a COX-2 enzyme such as osteoarthritic pain, and the capsules containing said invention compound, or said salt thereof, can be orally administered from 1 to 4 times per day to a mammal, including a human, suffering from a disease mediated by an MMP-13 enzyme such as osteoarthritic cartilage damage. The administrations may be performed substantially simultaneously or at different times.

Still another aspect is any one of said pharmaceutical compositions wherein the compound of Formula I, or a pharmaceutically acceptable salt thereof, is a compound of any one of Formulas II-XI, or a pharmaceutically acceptable salt thereof.

Another aspect of this invention is a method of inhibiting an MMP-13 enzyme in a mammal in need thereof comprising administering to the mammal an MMP-13 inhibiting amount of a compound of Formula I, or a pharmaceutically acceptable salt thereof. Another aspect of this invention is a method of inhibiting an MMP-13 enzyme in a mammal in need thereof, comprising administering to the mammal an MMP-13 inhibiting amount of a crystal form of a compound of Formula I, or a pharmaceutically acceptable salt thereof. Another aspect of this invention is a method of inhibiting an MMP-13 enzyme in a mammal in need thereof, comprising administering to the mammal an MMP-13 inhibiting amount of an invention pharmaceutical composition.

Still another aspect is any one of said methods of inhibiting an MMP-13 enzyme in a mammal in need thereof, wherein the compound of Formula I, or a pharmaceutically acceptable salt thereof, is a compound of any one of Formulas II-XI, or a pharmaceutically acceptable salt thereof.

Another aspect of this invention is a method of treating a disease mediated by an MMP-13 enzyme in a mammal in need thereof, comprising administering to the mammal a therapeutically effective amount of a compound of Formula I, or a pharmaceutically acceptable salt thereof.

Another aspect of this invention is a method of treating a disease mediated by an MMP-13 enzyme in a mammal in need thereof, comprising administering to the mammal a therapeutically effective amount of a combination, comprising a compound of Formula I, or a pharmaceutically acceptable salt thereof, together with another pharmaceutically active component as described herein.

Another aspect of this invention is a method of treating a disease mediated by an MMP-13 enzyme in a mammal in need thereof, comprising administering to the mammal a pharmaceutical composition, comprising a compound of Formula I, or a pharmaceutically acceptable salt thereof, together with a pharmaceutically acceptable carrier, diluent, or excipient.

Another aspect of this invention is any one of said methods of treating a disease mediated by an MMP-13 enzyme in a mammal in need thereof, wherein said disease is selected from the group consisting of osteoarthritis, rheumatoid arthritis, joint cartilage damage, heart failure abdominal aortic aneurysms, skin ulcers, and a cancer selected from the group consisting of: ovarian cancer, squamous carcinoma, head carcinoma, neck carcinoma, fibrosarcoma, chondrosarcoma, basal cell carcinoma of the skin, and breast cancer. Still another aspect of this invention is any one of said methods of treating a disease mediated by an MMP-13 enzyme in a mammal in need thereof, wherein said disease is selected from the group consisting of reactive arthritis, infectious arthritis, gouty arthritis, psoriatic arthritis, ankylosing spondylitis, multiple sclerosis, inflammatory bowel disease, age-related macular degeneration, chronic obstructive pulmonary disease, asthma, periodontal diseases, psoriasis, atherosclerosis, and osteoporosis.

Another aspect of this invention is any one of said methods of treating a disease mediated by an MMP-13 enzyme in a mammal in need thereof, wherein said disease is osteoarthritis.

Another aspect of this invention is any one of said methods of treating a disease mediated by an MMP-13 enzyme in a mammal in need thereof, wherein said disease is rheumatoid arthritis.

Another aspect of this invention is any one of said methods of treating a disease mediated by an MMP-13 enzyme in a mammal in need thereof, wherein said disease is joint cartilage damage.

Another aspect of this invention is any one of said methods of treating a disease mediated by an MMP-13 enzyme in a mammal in need thereof, wherein said disease is heart failure.

Still another aspect of this invention is any one of said methods of treating a disease mediated by an MMP-13 enzyme in a mammal in need thereof, wherein said disease is selected from the group consisting of osteoarthritis, rheumatoid arthritis, psoriatic arthritis, juvenile arthritis, reactive arthritis, Lyme arthritis, and infectious arthritis.

Still another aspect of this invention is a method of treating a joint disorder selected from the group consisting of joint pain, joint inflammation, joint edema, and impaired joint function in a mammal in need thereof, comprising administering to the mammal a therapeutically effective amount of a compound of Formula I, or a pharmaceutically acceptable salt thereof.

Still other aspects of this invention is a method of alleviating joint pain selected from the group consisting of acute joint pain, chronic joint pain, osteoarthritic joint pain, rheumatoid arthritic joint pain, post-operative joint pain, perioperative joint pain, and inflammatory joint pain in a mammal in need thereof, comprising administering to the mammal a therapeutically effective amount of a compound of Formula I, or a pharmaceutically acceptable salt thereof.

Still another aspect of this invention is a method of treating joint cartilage damage in a mammal in need thereof, comprising administering to the mammal a therapeutically effective amount of a compound of Formula I, or a pharmaceutically acceptable salt thereof.

Still another aspect of this invention is a method of treating fibromyalgia or a fibromyalgic symptom selected from the group consisting of fibromyalgic pain, sleep disturbance, and fatigue in a mammal in need thereof, comprising administering to the mammal a therapeutically effective amount of a compound of Formula I, or a pharmaceutically acceptable salt thereof.

Still another aspect of this invention is a method of treating an inflammatory skin disease or disorder selected from the group consisting of: psoriasis, eczema, atopic dermatitis, contact dermatitis, discoid lupus, pemphigus vulgaris, bullous pemphigoid, and alopecia areata in a mammal in need thereof, comprising administering to the mammal a therapeutically effective amount of a compound of Formula I, or a pharmaceutically acceptable salt thereof.

Still another aspect of this invention is a method of treating a skin ulcer or wound in a mammal in need thereof, comprising administering to the mammal a therapeutically effective amount of a compound of Formula I, or a pharmaceutically acceptable salt thereof.

Still another aspect of this invention is a method of alleviating pain selected from the group consisting of migraine, spinal pain, fibromyalgic pain, osteoarthritic pain, rheumatoid arthritic pain, and inflammatory pain in a mammal in need thereof, comprising administering to the mammal a therapeutically effective amount of a compound of Formula I, or a pharmaceutically acceptable salt thereof.

Another aspect of this invention is any one of said methods of treating a disease mediated by an MMP-13 enzyme in a mammal in need thereof, wherein the compound of Formula I, or a pharmaceutically acceptable salt thereof, is a crystal form of a compound of Formula I, or a pharmaceutically acceptable salt thereof. Still another aspect is any one of said methods of treating a disease mediated by an MMP-13 enzyme in a mammal in need thereof, wherein the compound of Formula I, or a pharmaceutically acceptable salt thereof, is a compound of any one of Formulas II-XI, or a pharmaceutically acceptable salt thereof. Still another aspect is any one of said methods of treating a disease mediated by an MMP-13 enzyme in a mammal in need thereof, wherein the compound of Formula I, or a pharmaceutically acceptable salt thereof, or any one of Formulas II-XI, or a pharmaceutically acceptable salt thereof, comprises a pharmaceutical composition.

Still further, it should be appreciated that the invention methods comprising administering an invention combination to a mammal to treat diseases or disorders listed above may be used to treat different diseases simultaneously. For example, administration of a COX-2 inhibitor in accordance with an invention combination may be carried out to treat, for example, inflammation, colon cancer, pain associated with menstrual cramping, or migraines, while said invention compound, or said salt thereof, or said crystal form, or said salt thereof, may be administered to treat, for example, cartilage damage due to osteoarthritis, heart failure, or abdominal aortic aneurysm.

Another aspect of this invention is a method of preparing a compound of Formula I, or a pharmaceutically acceptable salt thereof, a crystal form of a compound of Formula I, or a pharmaceutically acceptable salt thereof, an invention combination, or a pharmaceutical composition, comprising said compound of Formula I, or the pharmaceutically acceptable salt thereof, or said crystal form of a compound of Formula I, or a pharmaceutically acceptable salt thereof, and a pharmaceutically acceptable diluent, carrier, or excipient, as described herein.

Another aspect of this invention is a process for preparing a compound of Formula I 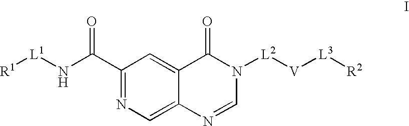
or a pharmaceutically acceptable salt thereof, wherein R¹, L¹, L², V, L³, and R² are as defined above,
comprising deprotecting a compound of Formula Ia 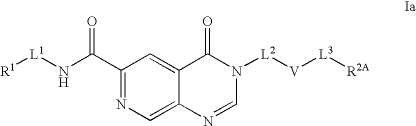
or a pharmaceutically acceptable salt thereof, and optionally converting the compound of Formula I produced thereby to a pharmaceutically acceptable salt thereof,
wherein:
R^{2A} is a radical independently selected from the group consisting of —SO₃R^{PG}, —PO₃(R^{PG})₂, —(CH₂)_{0 or 1}—N(R^{PG})-G-R, —C(O)N(R^{PG})-G-R, -G-N(R^{PG})—C(O)—R, and a 5-membered heterocycle radical selected from the group consisting of: 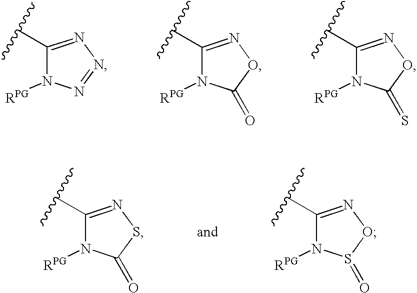
   and
R^{2A} may further be a radical independently selected from the group consisting of —CO₂R^{PG}, when L³ is absent or is a diradical independently selected from the group consisting of CH₂, CH₂CH₂, OCH₂, N(H)CH₂, SCH₂, S(O)CH₂, S(O)₂CH₂,
wherein said L³ diradicals are unsubstituted or substituted with 1 or 2 substituents R^{X}; and
wherein R^{PG} is a suitable protecting group and R¹, L¹, L², V, L³, and R^{X} are as defined above for Formula I.

Still another aspect of this invention is said process for preparing the compound of Formula I, or a pharmaceutically acceptable salt thereof, wherein when R^{2A} is —SO₃R^{PG}, —P₃(R^{PG})₂, —(CH₂)_{0 or 1}—N(R^{PG})-G-R, —C(O)N(R^{PG})-G-R, —G-N(R^{PG})—C(O)—R, or 5-membered heterocycle radical, R^{PG} is methyl, tertiary butyl, trityl, diphenylmethyl, benzyl, or 4-methoxybenzyl; and
wherein when R^{2A} is —CO₂R^{PG}, R^{PG} is C₁-C₁₀ alkyl, benzyl, diphenylmethyl, or trityl, wherein said C₁-C₁₀ alkyl, benzyl, diphenylmethyl, or trityl are unsubstituted or substituted by from 1 to 3 substituents R^{X} and wherein C₁-C₁₀ alkyl is an acyclic hydrocarbon radical containing from 1 to 10 carbon atoms in a straight or branched configuration.

Still another aspect of this invention is a process for preparing a compound of Formula II 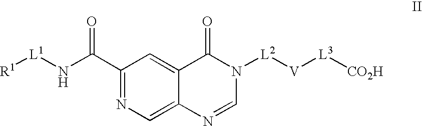
or a pharmaceutically acceptable salt thereof, comprising deprotecting a compound of formulas IIa or IIIb 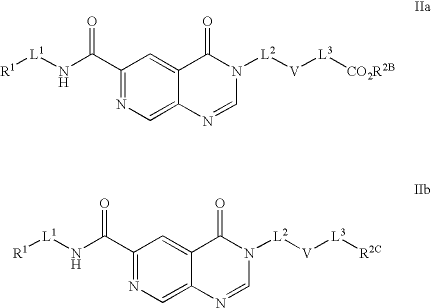
or a pharmaceutically acceptable salt thereof, and optionally converting the compound of Formula I produced thereby to a pharmaceutically acceptable salt thereof,
wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, wherein said L³ diradicals are unsubstituted or substituted with 1 or 2 substituents R^{X}, wherein R^{X} is as defined above for Formula I;
R^{2B} is a carboxylic acid protecting group selected from the group consisting of C₁-C₁₀ alkyl, benzyl, (C₁-C₁₀ alkyl)₃Si, allyl, and cinnamyl, wherein said C₁-C₁₀ alkyl, C₃-C₁₀ cycloalkyl, benzyl, (C₁-C₁₀ alkyl)₃Si, allyl, and cinnamyl are unsubstituted or substituted with from 1 to 3 substituents selected from the group consisting of F, Cl, Br, I, NO₂, CH₃, (C₁-C₆ alkyl)-O, phenyl, 4-methoxyphenyl, (C₁-C₆ alkyl)₃Si, phenylsulfonyl 4-methylphenylsulfonyl, and 4-nitrobenzylsulfonyl; and 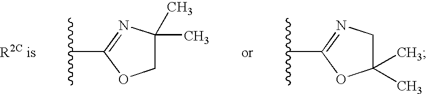
wherein C₁-C₁₀ alkyl is an acyclic hydrocarbon radical containing from 1 to 10 carbon atoms in a straight or branched configuration and C₃-C₁₀ cycloalkyl is a carbocyclic radical containing from 3 to 10 carbon atoms.

Still another aspect of this invention is said process for preparing the compound of Formula II, or a pharmaceutically acceptable salt thereof, wherein R^{2B} is selected from the group consisting of unsubstituted C₁-C₁₀ alkyl, unsubstituted C₃-C₁₀ cycloalkyl, 2,2,2-trichloroethyl, 2-trimethylsilyl-ethyl, 2-(di(normal-butyl)methylsilyl)ethyl, 2-(para-toluenesulfonyl)-ethyl, 2-(4-nitrobenzylsulfonyl)-ethyl, benzyl, 4-nitrobenzyl, 2-, 3-, and 4-methoxybenzyl, 2,3-, 2,4-, 2,5- 2,6- 3,4-, and 3,5-dimethoxybenzyl, 2,4,6-trimethoxybenzyl, 2-, 3-, and 4-methylbenzyl, 2,3-, 2,4-, 2,5- 2,6- 3,4-, and 3,5-dimethylbenzyl, 2,4,6-trimethylbenzyl, 2,3,4,6-tetramethylbenzyl, 2,3,4,5,6-pentamethylbenzyl, 3,4-methylenedioxybenzyl, benzhydryl, diphenylmethyl, 4-methoxydiphenylmethyl, 4,4′-dimethoxydiphenylmethyl, trityl, 4-methoxytrityl, 4,4′-dimethoxytrityl, 4,4′,4″-trimethoxytrityl, 2-phenyl-prop-2-yl, trimethylsilyl, tertiary-butyl-dimethylsilyl, allyl, cinnamyl, and 1-(trimethylsilylmethyl)-prop-1-en-3-yl, and the like.

Still another aspect of this invention is said process for preparing the compound of Formula II, or a pharmaceutically acceptable salt thereof, wherein R^{2B} is selected from the group consisting of methyl, ethyl, propyl, iso-propyl, normal-butyl, secondary-butyl, iso-butyl, tertiary-butyl, normal-pentyl, secondary-pentyl, 3-pentyl, 1,1-dimethylpropyl, normal-hexyl, normal-heptyl, normal-octyl, normal-nonyl, normal-decyl, 2,2,2-trichloroethyl, 2-trimethylsilyl-ethyl, 2-(di(normal-butyl)methylsilyl)ethyl, 2-(para-toluenesulfonyl)-ethyl, 2-(4-nitrobenzylsulfonyl)-ethyl, benzyl, 4-nitrobenzyl, 2-, 3-, and 4-methoxybenzyl, 2,3-, 2,4-, 2,5- 2,6- 3,4-, and 3,5-dimethoxybenzyl, 2,4,6-trimethoxybenzyl, 2-, 3-, and 4-methylbenzyl, 2,3-, 2,4-, 2,5- 2,6- 3,4-, and 3,5-dimethylbenzyl, 2,4,6-trimethylbenzyl, 2,3,4,6-tetramethylbenzyl, 2,3,4,5,6-pentamethylbenzyl, 3,4-methylenedioxybenzyl, benzhydryl, diphenylmethyl, 4-methoxydiphenylmethyl, 4,4′-dimethoxydiphenylmethyl, trityl, 4-methoxytrityl, 4,4′-dimethoxytrityl, 4,4′,4″-trimethoxytrityl, 2-phenyl-prop-2-yl, trimethylsilyl, tertiary-butyl-dimethylsilyl, allyl, cinnamyl, and 1-(trimethylsilylmethyl)-prop-1-en-3-yl, and the like.

Still another aspect of this invention is said process for preparing the compound of Formula II, or a pharmaceutically acceptable salt thereof, wherein R^{2B} is selected from the group consisting of methyl, ethyl, propyl, iso-propyl, normal-butyl, secondary-butyl, iso-butyl, tertiary-butyl, normal-pentyl, secondary-pentyl, 3-pentyl, 1,1-dimethylpropyl, 2,2,2-trichloroethyl, 2-trimethylsilyl-ethyl, 2-(di(normal-butyl)methylsilyl)ethyl, 2-(para-toluenesulfonyl)-ethyl, 2-(4-nitrobenzylsulfonyl)-ethyl, benzyl, 4-nitrobenzyl, 2-, 3-, and 4-methoxybenzyl, 2,3-, 2,4-, 2,5- 2,6- 3,4-, and 3,5-dimethoxybenzyl, 2,4,6-trimethoxybenzyl, 2-, 3-, and 4-methylbenzyl, 2,3-, 2,4-, 2,5- 2,6- 3,4-, and 3,5-dimethylbenzyl, 2,4,6-trimethylbenzyl, 3,4-methylenedioxybenzyl, benzhydryl, diphenylmethyl, 4-methoxydiphenylmethyl, 4,4′-dimethoxydiphenylmethyl, trityl, 4-methoxytrityl, 4,4′-dimethoxytrityl, 4,4′,4″-trimethoxytrityl, 2-phenyl-prop-2-yl, trimethylsilyl, tertiary-butyl-dimethylsilyl, allyl, cinnamyl, and 1-(trimethylsilylmethyl)-prop-1-en-3-yl, and the like.

Still another aspect of this invention is any one of said processes for preparing the compound of Formula II, or a pharmaceutically acceptable salt thereof, wherein the deprotection step comprises an acid catalyzed hydrolysis reaction. Still another aspect of this invention is any one of said processes for preparing the compound of Formula II, or a pharmaceutically acceptable salt thereof, wherein the deprotection step comprises an acid-catalyzed cleavage reaction. Still another aspect of this invention is any one of said processes for preparing the compound of Formula II, or a pharmaceutically acceptable salt thereof, wherein the deprotection step comprises a hydroxide base catalyzed hydrolysis reaction. Still another aspect of this invention is any one of said processes for preparing the compound of Formula II, or a pharmaceutically acceptable salt thereof, wherein the deprotection step comprises a hydrogenolysis reaction. Still another aspect of this invention is any one of said processes for preparing the compound of Formula II, or a pharmaceutically acceptable salt thereof, wherein the deprotection step comprises a fluoride ion catalyzed cleavage reaction. Still another aspect of this invention is any one of said processes for preparing the compound of Formula II, or a pharmaceutically acceptable salt thereof, wherein the deprotection step comprises a reductive cleavage reaction, especially wherein the reducing reagents comprise zinc metal in aqueous acetic acid. Still another aspect of this invention is any one of said processes for preparing the compound of Formula II, or a pharmaceutically acceptable salt thereof, wherein the deprotection step comprises a base catalyzed 1,2-elimination reaction. Suitable acid catalysts include hydrogen chloride, trifluoroacetic acid, acetic acid, propanoic acid, sulfuric acid, phosphoric acid, hydrochloric acid, and the like. Suitable solvents include acetonitrile, tetrahydrofuran, dioxane, ethyl ether, ethyl acetate, dichloromethane, dichloroethane, methanol, ethanol, propanol, isopropanol, acetone, cyclohexanone, dimethylformamide, dimethylsulfoxide, acetic acid, water, and the like, and mixtures thereof.

Still another aspect of this invention is any one of said processes for preparing the compound of Formula II, or a pharmaceutically acceptable salt thereof, wherein a compound of Formula IIa wherein R^{B} is tertiary-butyl is deprotected with an acid comprising trifluoroacetic acid in a solvent comprising acetonitrile.

Still another aspect of this invention is a process for preparing a compound of Formula II 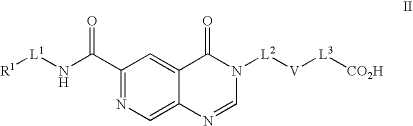
or a pharmaceutically acceptable salt thereof, comprising hydrolyzing a compound of formula IIc 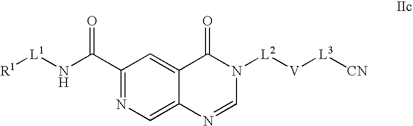
or a pharmaceutically acceptable salt thereof, and optionally converting the compound of Formula I produced thereby to a pharmaceutically acceptable salt thereof,
wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, wherein said L³ diradicals are unsubstituted or substituted with 1 or 2 substituents R^{X}, wherein R^{X} is as defined above for Formula I.

Still another aspect of this invention is a process for preparing a compound of Formula II 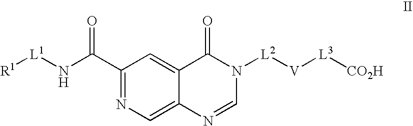
or a pharmaceutically acceptable salt thereof, comprising coupling a compound of formula (1) 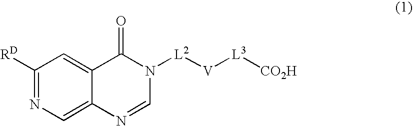
or a pharmaceutically acceptable salt thereof,
with carbon monoxide and a compound of formula (2)
   R¹-L¹-NH₂ (2)
   in the presence of a carbonylation catalyst and a non-nucleophilic base in a suitable solvent; and optionally converting the compound of Formula I produced thereby to a pharmaceutically acceptable salt thereof,
wherein R¹, L¹, L², and V, are as defined above for Formula I and L³ is absent or is a diradical independently selected from the group consisting of CH₂, CH₂CH₂, OCH₂, N(H)CH₂, SCH₂, S(O)CH₂, S(O)₂CH₂, wherein said L³ diradicals are unsubstituted or substituted with 1 or 2 substituents R^{X}, wherein R^{X} is as defined above for Formula I; and
R^{D} is Cl or Br.

Still another aspect of this invention is said process for preparing the compound of Formula II, or a pharmaceutically acceptable salt thereof, wherein the carbonylation catalyst is 1,1′-bis(diphenylphosphino)ferrocene dichloropalladium(II) or palladium acetate 1,3-bis(diphenylphosphino)propane, the non-nucleophilic base is triethylamine, and the solvent is tetrahydrofuran.

Still another aspect of this invention is said processes for preparing the compound of Formulas I or II, or a pharmaceutically acceptable salt thereof, wherein the compound of Formulas I and II is 4-[6-(4-methoxy-benzylcarbamoyl)-4-oxo-4H-pyrido[3,4-d]pyrimidin-3-ylmethyl]-benzoic acid, or a pharmaceutically acceptable salt thereof.

Another aspect of this invention is a process for preparing a compound of Formula I 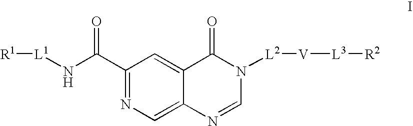
or a pharmaceutically acceptable salt thereof, comprising the step coupling a compound of formula (1) 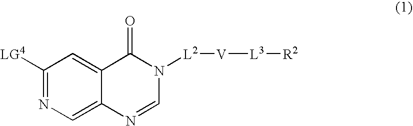
with carbon monoxide and a compound of formula (2)
   R¹-L¹-NH₂ (2)
   in the presence of a carbonylation catalyst, and optionally converting the compound of Formula I produced thereby to a pharmaceutically acceptable salt thereof, wherein R¹, L¹, L², V, L³, and R² are as defined above for Formula I.

Still another aspect of this invention is said process for preparing the compound of Formula I, or a pharmaceutically acceptable salt thereof, wherein the carbonylation catalyst is a 1,1′-bis(diphenylphosphino)ferrocene dichloropalladium(II) or palladium acetate 1,3-bis(diphenylphosphino)propane, and the solvent is tetrahydrofuran.

Still another aspect of this invention is said processes for preparing the compound of Formula I, or a pharmaceutically acceptable salt thereof, wherein the compound of Formula I is 4-[6-(4-methoxy-benzylcarbamoyl)-4-oxo-4H-pyrido[3,4-d]pyrimidin-3-ylmethyl]-benzoic acid, or a pharmaceutically acceptable salt thereof.

Another aspect of this invention is a process for preparing a compound of Formula I 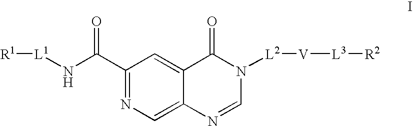
or a pharmaceutically acceptable salt thereof, comprising the step coupling a compound of formula (1) 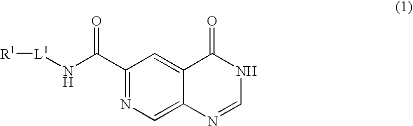
with a compound of formula (2)
   R¹-L²-V-L³-R² (2)
   in the presence of a suitable base, and optionally converting the compound of Formula I produced thereby to a pharmaceutically acceptable salt thereof,
wherein R^{E} is a leaving group or R^{E}-L²- are taken together to form an imine selected from the group consisting of CH₂═N— or CH(R^{X})═N— and R¹, L¹, L², V, L³, and R² are as defined above for Formula I.

Still another aspect of this invention is said process for preparing the compound of Formula I, or a pharmaceutically acceptable salt thereof, wherein R^{E} is selected from the group consisting of Cl, Br, I, CH₃SO₃, CF₃SO₃, and 4-R-phenyl-SO₃—, wherein R is H, CH₃, Br, CH₃O, and the like.

Still another aspect of this invention is said processes for preparing the compound of Formula I, or a pharmaceutically acceptable salt thereof, wherein the compound of Formula I is 4-[6-(4-methoxy-benzylcarbamoyl)-4-oxo-4H-pyrido[3,4-d]pyrimidin-3-ylmethyl]-benzoic acid, or a pharmaceutically acceptable salt thereof.

Another aspect of this invention is a method of determining the pharmacologic effect of a compound of Formula I, or a pharmaceutically acceptable salt thereof, or a crystal form of a compound of Formula I, or a pharmaceutically acceptable salt thereof, an invention combination, or an invention pharmaceutical composition, in a laboratory mammal, comprising administering to the mammal an MMP-13 enzyme inhibiting amount of a compound of Formula I, or a pharmaceutically acceptable salt thereof, or a crystal form of a compound of Formula I, or a pharmaceutically acceptable salt thereof, or a said invention combination. Another aspect of this invention is a method of determining the pharmacologic effect of a combination of a Formula I, or a pharmaceutically acceptable salt thereof, and a COX-2 inhibitor in a laboratory mammal, comprising administering to the laboratory mammal a therapeutically effective amount of said combination. Another aspect of this invention is a method of determining the pharmacologic effect of a combination of a Formula I, or a pharmaceutically acceptable salt thereof, and a biologic therapeutic agent selected from the group consisting of CP-870, etanercept, infliximab, methotrexate, and adalimumab in a laboratory mammal, comprising administering to the laboratory mammal a therapeutically effective amount of said combination.

Another aspect of this invention is an MMP-13 inhibitor selected from the group consisting of:
4-[6-(4-methoxy-benzylcarbamoyl)-4-oxo-4H-pyrido[3,4-d]pyrimidin-3-ylmethyl]-benzoic acid tert-butyl ester;
4-[6-(3-methoxy-benzylcarbamoyl)-4-oxo-4H-pyrido[3,4-d]pyrimidin-3-ylmethyl]-benzoic acid tert-butyl ester;
4-{4-oxo-6-[(pyridin-3-ylmethyl)-carbamoyl]-4H-pyrido[3,4-d]pyrimidin-3-ylmethyl}-benzoic acid tert-butyl ester;
4-[6-(4-chloro-benzylcarbamoyl)-4-oxo-4H-pyrido[3,4-d]pyrimidin-3-ylmethyl]-benzoic acid tert-butyl ester;
4-{4-oxo-6-[(pyridin-4-ylmethyl)-carbamoyl]-4H-pyrido[3,4-d]pyrimidin-3-ylmethyl}-benzoic acid tert-butyl ester;
4-{6-[(2-methoxy-pyridin-4-ylmethyl)-carbamoyl]-4-oxo-4H-pyrido[3,4-d]pyrimidin-3-ylmethyl}-benzoic acid tert-butyl ester;
4-[6-(4-methylsulfanyl-benzylcarbamoyl)-4-oxo-4H-pyrido[3,4-d]pyrimidin-3-ylmethyl]-benzoic acid tert-butyl ester;
4-[6-(4-fluoro-benzylcarbamoyl)-4-oxo-4H-pyrido[3,4-d]pyrimidin-3-ylmethyl]-benzoic acid tert-butyl ester;
4-[6-benzylcarbamoyl)-4-oxo-4H-pyrido[3,4-d]pyrimidin-3-ylmethyl]-benzoic acid tert-butyl ester;
4-[6-(3-chloro-benzylcarbamoyl)-4-oxo-4H-pyrido[3,4-d]pyrimidin-3-ylmethyl]-benzoic acid tert-butyl ester;
4-[6-(3-fluoro-benzylcarbamoyl)-4-oxo-4H-pyrido[3,4-d]pyrimidin-3-ylmethyl]-benzoic acid tert-butyl ester;
4-[4-oxo-6-(4-trifluoromethyl-benzylcarbamoyl)-4H-pyrido[3,4-d]pyrimidin-3-ylmethyl]-benzoic acid tert-butyl ester;
4-[4-oxo-6-(3-trifluoromethyl-benzylcarbamoyl)-4H-pyrido[3,4-d]pyrimidin-3-ylmethyl]-benzoic acid tert-butyl ester;
4-6-(3,4-difluoro-benz ylcarbamoyl)-4-oxo-4H-pyrido[3,4-d]pyrimidin-3-ylmethyl]-benzoic acid tert-butyl ester;
4-[6-(4-hydroxy-3-methoxy-benzylcarbamoyl)-4-oxo-4H-pyrido[3,4-d]pyrimidin-3-ylmethyl]-benzoic acid tert-butyl ester; and
4-[6-(4methoxy-benzylcarbamoyl)-4-oxo-4H-pyrido-[3,4-d]pyrimidin-3-ylmethyl]-cyclohexanecarboxylic acid methyl ester; or
a pharmaceutically acceptable salt thereof.

Another aspect of this invention is an intermediate selected from the group consisting of:
4-(6-chloro-4-oxo-4H-pyrido[3,4-d]pyrimidin-3-ylmethyl)-benzoic acid tert-butyl ester;
3-(4-tert-butoxycarbonyl-benzyl)-4-oxo-3,4 dihydro-pyrido[3,5-d]pyrimidine-6-carboxylic acid methyl ester; and
4-oxo-3,4-dihydro-pyrido[3,4-d]pyrimidine-6-carboxylic acid 4-methoxy-benzylamide; or
a pharmaceutically acceptable salt thereof.

### BRIEF DESCRIPTION OF THE DRAWINGS

FIG. 1 is a powder X-ray diffractogram of an powder x-ray diffraction pattern that was collected with Crystal Form 1 of 4-[6-(4-methoxy-benzylcarbamoyl)-4-oxo-4H-pyrido[3,4-d]pyrimidin-3-ylmethyl]-benzoic acid using a Bruker D8 powder X-ray diffractometer utilizing a copper target.

FIG. 2 is a powder X-ray diffractogram of an powder x-ray diffraction pattern that was collected with Crystal Form 1 of 4-[6-(4-methoxy-benzylcarbamoyl)-4-oxo-4H-pyrido[3,4-d]pyrimidin-3-ylmethyl]-benzoic acid using a Rigaku powder X-ray diffractometer utilizing a copper target.

### DETAILED DESCRIPTION OF THE INVENTION

As mentioned above, one aspect of this invention is a compound of Formula I 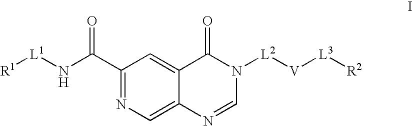
or a pharmaceutically acceptable salt thereof,
wherein R¹, L¹, L², V, L³, and R² are as defined above.
   Definitions:

As defined above, a compound of Formula I includes 5- and 6-membered heteroaryl, 9- and 10-membered heterobiaryl, phenylene, naphthylene, 5- and 6-membered heteroarylene, 9- and 10-membered heterobiarylene, C₃-C₆ cycloalkylene, 3- to 6-membered heterocycloalkylene, C₆-C₁₀ bicycloalkylene, 6- to 10-membered heterobicycloalkylene, C₁-C₆ alkyl, 2- to 6-membered heteroalkyl, C₃-C₅ cycloalkyl, and 3- to 5-membered heterocycloalkyl groups, which may be unsubstituted or substituted. A compound of Formula Ia also includes C₁-C₁₀ alkyl groups.

Illustrative examples of C₁-C₆ alkyl groups include methyl, ethyl, 1-propyl, 2-propyl, 1-butyl, 2-butyl, 2,2-dimethylethyl, 1-pentyl, 2-pentyl, 2,2-dimethylpropyl, 1-hexyl, and the like. Illustrative examples of substituted C₁-C₆ alkyl groups include CF₃, CH₂OH, CF₂OH, CH₂C(CH₃)₂CO₂CH₃, CF₃, C(O)CF₃, C(O)—CH₃, (CH₂)₄—S—CH₃, CH(CO₂H)CH₂CH₂C(O)NMe₂, (CH₂)₅NH—C(O)—NH₂, CH₂—CH₂—C(H)—(4-fluorophenyl), CH(OCH₃)CH₂CH₃, CH₂SO₂NH₂, CH(CH₃)CH₂CH₂OC(O)CH₃, and the like.

Illustrative examples of C₁-C₁₀ alkyl groups include the C₁-C₆ alkyl groups recited above as well as the following illustrative C₇-C₁₀ alkyl groups: 1-heptyl, 2-octyl, 5-nonyl, and 3,3-diethyl-hex-1-yl.

Illustrative examples of 2- to 6-membered heteroalkyl groups include CH₃N(H), NH₂CH₂, CH₃OCH₂CH₂, (CH₃)₃CSCH₂, (CH₃)₂C(H)OCH₂the like. Illustrative examples of substituted 2- to 6-membered heteroalkyl groups include CH₃N(CH₃), NC—NHCH₂, CH₃OC(O)CH₂, (CH₃)₃CS(O)C(H)—C(O)N(CH₃)₂, (CF₃)₂C(H)OCH₂N(H), CH₃S(O)₂, and the like.

Illustrative examples of C₃-C₅ cycloalkyl include cyclopropyl, cyclobutyl, cyclopentyl, cyclopenten-1-yl, cyclopenten-4-yl, and the like. Illustrative examples of substituted C₃-C₅ cycloalkyl include 1-fluorocyclopropyl, 3-carboxycyclobutyl, 2-oxocyclopentyl, 2-dimethylaminocyclopenten-1-yl, 4-hydroxycyclopenten-4-yl, and the like.

Illustrative examples of 3- to 5-membered heterocycloalkyl include aziridin-2-yl, 3-thiacyclobutyl, tetrahydrofuran-2-yl, 2-azacyclopenten-1-yl, 4,5-dihydroisoxazol-3-yl, and the like. Illustrative examples of substituted 3- to 5-membered heterocycloalkyl include 2-oxoaziridin-1-yl, 2,2-difluoro-3-thiacyclobutyl, 2-carboxypyrrollidin-1-yl, 4-oxo-3-azacyclopenten-1-yl, 4-acetoxy-4,5-dihydroisoxazol-3-yl, 1,1-dioxo-tetrahydrothien-2-yl, and the like.

Illustrative examples of a 5-membered heteroaryl include thiophen-2-yl, furan-2-yl, pyrrol-3-yl, pyrrol-1-yl, imidazol-4-yl, isoxazol-3-yl, oxazol-2-yl, thiazol-4-yl, tetrazol-1-yl, 1,2,4-oxadiazol-3-yl, 1,2,4-triazol-1-yl, pyrazol-3-yl, and the like. Illustrative examples of a substituted 5-membered heteroaryl include 5-carboxy-thiophen-2-yl, 3-chloro-furan-2-yl, 2-hydroxy-oxoazol-4-yl, 5-chloro-thiophen-2-yl, 1-methylimidazol-5-yl, 1-propyl-pyrrol-2-yl, 1-acetyl-pyrazol-4-yl, 1-methyl- 1,2,4-triazol-3-yl, 2-hexyl-tetrazol-5-yl, and the like.

Illustrative examples of a 6-membered heteroaryl include pyridin-2-yl, pyridin-4-yl, pyrimidin-2-yl, pyridazin-4-yl, pyrazin-2-yl, and the like. Illustrative examples of substituted 6-membered heteroaryt groups include 4-acetyl-pyridin-2-yl, 3-fluoro-pyridin-4-yl, 5-carboxy-pyrimidin-2-yl, 6-tertiary butyl-pyridazin-4-yl, 5-hdyroxymethyl-pyrazin-2-yl, and the like.

Additional illustrative examples of 5- and 6-membered heteroaryl groups include, isothiazolyl, isoxazolyl, oxadiazolyl, oxazolyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl; isothiazolyl, isoxazolyl, oxadiazolyl, oxazolyl, purinyl, pyrazinyl, pyridazinyl, pyridinyl, pyrimidinyl, pyrazolyl, quinazolinyl, quinolinyl, quinoxalinyl, tetrazolyl, thiazolyl, thiadiazolyl, thienyl, triazinyl, and triazolyl; oxazolyl, purinyl, pyrazinyl, pyridazinyl, pyridinyl, pyrimidinyl, pyrazolyl, pyrrolyl, quinazolinyl, quinolinyl, quinoxalinyl, tetrazolyl, thiazolyl, thiadiazolyl, thienyl, triazinyl, and triazolyl; isothiazolyl, oxadiazolyl, purinyl, pyrazinyl, pyridazinyl, pyridinyl, pyrazolyl, tetrazolyl, thiazolyl, thiadiazolyl, triazinyl, and triazolyl; isothiazolyl, isoxazolyl, and oxadiazolyl; oxazolyl and purinyl; isoxazolyl and oxadiazolyl; tetrazolyl; thiazolyl; thiadiazolyl; thienyl; and triazolyl.

Illustrative examples of a 9-membered heterobiaryl include indol-2-yl, indol-6-yl, iso-indol-2-yl, benzimidazol-2-yl, benzimidazol-1-yl, benztriazol-1-yl, benztriazol-5-yl, benzoxazol-2-yl, benzothiophen-5-yl, benzofuran-3-yl, and the like. Illustrative examples of substituted 9-membered heterobiaryl include 3-(2-aminomethyl)-indol-2-yl, 2-carboxy-indol-6-yl, 1-(methanesulfonyl)-iso-indol-2-yl, 5-trifluorometyl-6,7-difluoro-4-hydroxymethyl-benzimidazol-2-yl, 4-(3-methylureido)-2-cyano-benzimidazol-1-yl, 1-methylbenzimidazol-6-yl, 1-acetylbenztriazol-7-yl, 1-methanesulfonyl-indol-3-yl, 1-cyano-6-aza-indol-5-yl, 1-(2,6-dichlorophenylmethyl)-benzpyrazol-3-yl, and the like.

Illustrative examples of a 10-membered heterobiaryl include quinolin-2-yl, isoquinolin-7-yl, benzopyrimidin-2-yl, and the like. Illustrative examples of substituted 10-membered heterobiaryl include 5,7-dichloro-quinolin-2-yl, isoquinolin-7-yl-1-carboxylic acid ethyl ester, 3-bromo-benzopyrimidin-2-yl, and the like.

Illustrative examples of a 5-membered heteroarylene include thiophen-2,4-diyl, furan-2,5-diyl, pyrrol-1,3-diyl, imidazol-1,4-diyl, pyrazol-3,5-diyl, and the like. Illustrative examples of a substituted 5-membered heteroarylene include 5-trifluoromethyl-thiophen-2,4-diyl, 4-methyl-furan-2,5-diyl, and the like.

Illustrative examples of a 6-membered heteroarylene include pyridin-2,4-diyl, pyridin-3,5-diyl, pyrimidin-2,5-diyl, pyridazin-3,6-diyl, pyrazin-2,5-di-yl, and the like. Illustrative examples of substituted 6-membered heteroarylene groups include 3-acetyl-pyridin-2,4-diyl, 2-fluoro-pyridin-3,5-diyl, 3-carboxy-pyrimidin-2,5-diyl, 5-tertiary butyl-pyridazin-3,6-diyl, 3-hydroxymethyl-pyrazin-2,5-di-yl, and the like.

Illustrative examples of a 9-membered heterobiarylene include indol-2,5-diyl, indol-1,6-diyl, iso-indol-2,5-diyl, benzimidazol-2,6-diyl, benzimidazol-1,3-diyl, benztriazol-1,4-diyl, benzoxazol-2,5-diyl, benzothiophen-4,7-diyl, benzofuran-3,5-diyl, and the like. Illustrative examples of substituted 9-membered heterobiarylene include 3-(2-aminomethyl)-indol-2,5-diyl, 2-carboxy-indol-1,6-diyl, 1-(methanesulfonyl)-iso-indol-2,5-diyl, 5,7-difluoro-4-hydroxymethyl-benzimidazol-2,6-diyl, 4-(3-methylureido)-2-cyano-benzimidazol-1,3-diyl, 2-trifluoromethylbenzothiophen-4,7-diyl, and the like.

Illustrative examples of a 10-membered heterobiarylene include quinolin-2,7-diyl, isoquinolin-3,6-diyl, isoquinolin-1,4-diyl, quinazolin-3,6-diyl, and the like. Illustrative examples of substituted 10-membered heterobiarylene include 3-fluoro-quinolin-2,7-diyl, 1-methoxy-isoquinolin-3,6-diyl, 3-hydroxyisoquinolin-1,4-diyl, 2-methyl-7-fluoroquinazolin-3,6-diyl, and the like.

Illustrative examples of C₃-C₆ cycloalkylene include cycloprop-1,2-diyl, cyclobut-1,3-diyl, cyclopent-1,3-diyl, cyclopenten-1,3-diyl, cyclohexen-1,4-diyl, and the like. Illustrative examples of substituted C₃-C₅ cycloalkylene include 1-fluoro-cycloprop-1,2-diyl, 3-carboxy-cyclobut-1,3-diyl, 2-oxo-cyclopent-1,3-diyl, 2-dimethylamino-cyclopenten-1,3-diyl, 3-hydroxy-cyclohexen-1,4-diyl, and the like.

Illustrative examples of 3- to 6-membered heterocycloalkylene include aziridin-1,2-diyl, 1-oxa-cyclobutan-1,3-diyl, tetrahyrdofuran-3,5-diyl, morpholin-2,4-diyl, 2-thiacyclohex-1,3-diyl, 2-oxo-2-thiacyclohex-1,4-diyl, 2,2-dioxo-2-thiacyclohex-1,5-diyl, 4-methyl-piperazin-2,5-diyl, and the like. Illustrative examples of substituted 3- to 6-membered heterocycloalkylene include 2-oxo-piperidin-1,4-diyl, 2,4-dihydro-pyrazol-3-one-1,4-diyl, and the like.

Illustrative examples of C₆-C₁₀ bicycloalkylene include bicyclo[2.2.0]hexan-2,5-diyl, bicyclo[3.2.0]heptan-2,4-diyl, bicyclo[3.3.0]octan-2,5-diyl, bicyclo[4.2.0]octan-2,4-diyl, bicyclo[4.3.0]nonan-2,6-diyl, bicyclo[4.4.0]decan-2,7-diyl, bicyclo[2.1.1]hexan-2,5-diyl, bicyclo[2.2.1]heptan-2,4-diyl, bicyclo[2.2.2]octan-2,5-diyl, bicyclo[3.2.2]nonan-2,6-diyl, adamantan-1,4-diyl, and the like. Illustrative examples of substituted C₆-C₁₀ bicycloalkylene include 1-fluoro-bicyclo[2.2.0]hexan-2,5-diyl, 5-oxo-bicyclo[2.1.1]hexan-2,4-diyl, and the like.

Illustrative examples of 6- to 10-membered heterobicycloalkylene 1-azabicyclo[2.2.0]hexan-2,5-diyl, 6-oxabicyclo[2.1.1]hexan-2,5-diyl, any one of the illustrative examples of C₆-C₁₀ bicycloalkylene described above wherein a CH₂ (sp³ carbon atom) is replaced with a heteroatom selected from the group consisting of O, S, S(O), S(O)₂, and N(H), any one of the illustrative examples of C₆-C₁₀ bicycloalkylene described above wherein a bridgehead CH is replaced with a N, and the like.

Illustrative examples of substituted phenylene include 2-fluoro-1,3-phenylene, 2-methoxy-1,4-phenylene, and the like.

Illustrative examples of substituted naphthylene include 2-fluoro-naphthylen-1,3-diyl, 5-methoxy-naphthylen-1,4-diyl, 7-trifluoromethyl-naphthylen-2,6-diyl, 6-hydroxy-naphthylen-2,7-diyl, and the like.

The term “heteroatom” includes O, S, S(O), S(O)₂, N, and N(H). Any N(H) may be substituted on the nitrogen with a group R^{X}, wherein R^{X} is as defined above as a nitrogen atom substituent.

It should be appreciated that a compound of Formula I does not contain contiguous oxygen and/or sulfur atoms.

It should be appreciated that phrases such as “from 1 to 4 heteroatoms independently selected from the group consisting of 1 O, 1 S, 1 N(H), and 3 N,” “from 1 to 4 heteroatoms independently selected from the group consisting of 1 O, 1 S, 1 N(H), and 4 N,” and the like mean independently 1, 2, 3, or 4 heteroatoms that form any combination selected from the group consisting of 1O, 1S, 1N(H), N, N, and N or 1O, 1S, 1N(H), N, N, N, and N, respectively, and the like. For example, a group of 4 heteroatoms may include 1O, 1S, and 2N; 1N(H) and 3N; 4N in the case of the second phrase; 1O, 1S, 1N(H), and 1N; and the like.

It should also be appreciated that any substituted group that has been defined as having a certain number of substituents R^{X} has a maximum number of substituents R^{X} equal to either the maximum number defined for said substituted group or the maximum number of protons in a corresponding unsubstituted group, whichever is lower.

It should be appreciated that the illustrative examples described above are not to be construed as limiting the invention in any aspect.

A fused bicyclic group is a group comprising two rings wherein the two ring systems share two, and only two, atoms.

A bridged bicyclic group is a group comprising two rings wherein the two ring systems share three or more atoms.

The term “oxo” means=O. Oxo is attached at a carbon atom unless otherwise noted. Oxo, together with the carbon atom to which it is attached forms a carbonyl group (i.e., C═O).

The term “halo” includes fluoro, chloro, bromo, and iodo.

The term “naphthyl” includes 1-naphthyl and 2-napthyl.

The term “leflunomide” includes the product marketed under the tradename ARAVA® registered to Hoechst Aktiengesellschaft, Frankfurt, Federal Republic of Germany.

The term “etanercept” includes a tumor necrosis factor alpha (“TNF-alpha”) receptor immunoglobulin molecule marketed under the tradenames ENBREL® and ENBREL ENTANERCEPT® registered to Immunex Corporation, Seattle, Wash.

The term “infliximab” includes an anti-TNF-alpha chimeric IgG 1K monoclonal antibody marketed under the tradename REMICADE® registered to Centocor, Inc., Malvern, Pa.

The term “methotrexate” includes the product marketed under the tradename RHEUMATREX® registered to American Cyanamid Company, Wayne, N.J.

The term “adalimumab” includes a human monoclonal anti-TNF-alpha antibody marketed under the tradename HUMIRA® registered to Abbott Laboratories, Abbott Park, Ill.

The phrase “pharmaceutical composition” means a composition suitable for administration to a mammal in any medical or veterinary use, not limited to those uses described herein.

The term “mammal” includes humans, companion animals such as cats, dogs, and the like, primates such as monkeys, chimpanzees, and the like, livestock animals such as horses, cows, pigs, sheep, and the like, and laboratory animals such as cats, dogs, rats, mice, guinea pigs, hamsters, rabbits, monkeys, pigs, and the like. A mammal includes wild type mammals and transgenic variants thereof.

The phrase “livestock animals” as used herein refers to domesticated quadrupeds, which includes those being raised for meat and various byproducts, e.g., a bovine animal including cattle and other members of the genus Bos, a porcine animal including domestic swine and other members of the genus Sus, an ovine animal including sheep and other members of the genus Ovis, domestic goats and other members of the genus Capra; domesticated quadrupeds being raised for specialized tasks such as use as a beast of burden, e.g., an equine animal including domestic horses and other members of the family Equidae, genus Equus, or for searching and sentinel duty, e.g., a canine animal including domestic dogs and other members of the genus Canis; and domesticated quadrupeds being raised primarily for recreational purposes, e.g., members of Equus and Canis, as well as a feline animal including domestic cats and other members of the family Felidae, genus Felis.

For the purposes of this invention, the term “arthritis”, which is synonymous with the phrase “arthritic condition”, includes osteoarthritis, rheumatoid arthritis, degenerative joint disease, spondyloarthropathies, gouty arthritis, systemic lupus erythematosus, juvenile arthritis, and psoriatic arthritis.

The phrase “MMP-13 inhibiting amount” means an amount of invention compound that is sufficient to achieve, upon intravenous administration of the compound to a mammal, a concentration of the compound in the mammal's blood, sampled at any time point, that is equal to or greater than the IC₅₀ concentration for the compound with human full-length MMP-13 when determined according to the method of Biological Example 1. In a human or other mammal, said MMP-13 inhibiting amount can be determined experimentally in a laboratory or clinical setting.

The term “IC₅₀” means the concentration of a compound, usually expressed as micromolar or nanomolar, required to inhibit an enzyme's catalytic activity by 50%.

The terms “ED₄₀” and “ED₃₀” mean the concentration of a compound, usually expressed as micromolar or nanomolar, required to treat a disease in about 40% and 30%, respectively, of a patient group.

As used herein, the phrase “cartilage damage” means a disorder of hyaline cartilage and subchondral bone characterized by hypertrophy of tissues in and around the involved joints, which may or may not be accompanied by deterioration of hyaline cartilage surface.

The phrase “impaired joint function” means less than full range of motion of a joint or less than normal weight bearing capacity of a joint. The phrase “joint function” relates to any one or more of the clinical assessments of joint function, including stiffness, range of movement, flexibility, and movement-related symptoms (e.g., altered gait, pain, warmth, or inflammation), in a patient suffering from any one of the diseases and disorders being improved, including, but not limited the diseases of rheumatoid arthritis and osteoarthritis. A clinician may use the Western Ontario and McMaster Universities Osteoarthritis Index (“WOMAC”) to assess joint function.

The phrase “treating”, which is related to the terms “treat” and “treated”, means administration of an invention combination as defined above that alleviates, inhibits the progress, prevents further progress, or reverses progression, in part or in whole, of any one or more pathologies or symptoms of any one of the diseases and disorders listed above.

The phrase “a mammal in need thereof” means a mammal currently afflicted with, or predicted to be afflicted with in the future, the disease for which the mammal is in need of treatment. Another aspect of this invention is preventing any single disease disclosed above, wherein said prevention is by prophylactic administration of an invention compound or pharmaceutical composition.

The term “patient” means a mammal as defined herein.

An invention combination or pharmaceutical composition may thus be administered prophylactically to prevent or inhibit, for example, the onset of osteoarthritis, rheumatoid arthritis, loss of joint function, cartilage damage, or any pain in an asymptomatic patient (mammal). It should be appreciated that an asymptomatic patient at risk for the disease or disorder being prevented may be identified by analysis of genetic risk factors (inherited or spontaneous mutation diseases and disorders), family medical history, occupation, participation in athletic activities, general medical screening, and the like.

The phrase “alleviating pain” means decreasing the severity, intensity, or longevity of the pain being alleviated.

The phrase “joint pain” means any pain in a joint.

The phrase “osteoarthritic pain” means joint pain in an osteoarthritic joint.

The phrase “rheumatoid arthritic pain” means joint pain in a rheumatoid arthritic joint.

The phrase “inflammatory pain” means pain in a tissue that also exhibits edema or swelling, including inflammatory joint pain. Inflammatory joint pain includes rheumatoid arthritic joint pain.

The phrase “acute pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, and dynamic allodynia, that lasts from 1 minute to 91 days, 1 minute to 31 days, 1 minute to 7 days, 1 minute to 5 days, 1 minute to 3 days, 1 minute to 2 days, 1 hour to 91 days, 1 hour to 31 days, 1 hour to 7 days, 1 hour to 5 days, 1 hour to 3 days, 1 hour to 2 days, 1 hour to 24 hours, 1 hour to 12 hours, or 1 hour to 6 hours, per occurrence if left untreated. Acute pain includes, but is not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, acute joint pain, acute osteoarthritic pain, acute rheumatoid arthritic pain, acute inflammatory pain, acute headache, acute menstrual pain, acute back pain, and acute pain from fibromyalgia. Acute pain may be selected from the group consisting of acute joint pain, acute osteoarthritic pain, acute rheumatoid arthritic pain, acute inflammatory pain, acute headache, acute menstrual pain, and acute back pain. Acute pain may be selected from the group consisting of acute joint pain, acute osteoarthritic pain, acute rheumatoid arthritic pain, and acute inflammatory pain. Acute pain may be selected from the group consisting of acute joint pain, acute osteoarthritic pain, and acute rheumatoid arthritic pain. Acute pain may be selected from the group consisting of acute joint pain and acute osteoarthritic pain.

It should be appreciated that alleviating acute pain means having an appreciable pain alleviating effect within 91, 31, 7, 5, 3, or 2 days, or 24, 12, 6, 3, 2, 1, 0.5, 0.25, 0.20. 0.17, or 0.10 hours after administering the first dose of an active ingredient.

The phrase “chronic pain” means any pain, including, but not limited to, joint pain, osteoarthritic pain, rheumatoid arthritic pain, inflammatory pain, pain from a burn, pain from a cut, surgical pain, pain from fibromyalgia, bone cancer pain, menstrual pain, back pain, headache, static allodynia, dynamic allodynia, that lasts longer than 91 days, 6 months, 1 year, 5 years, or 10 years per occurrence if left untreated. Chronic pain may be selected from the group consisting of chronic joint pain, chronic osteoarthritic pain, chronic rheumatoid arthritic pain, chronic inflammatory pain, chronic headache, chronic menstrual pain, chronic back pain, and chronic pain from fibromyalgia. Chronic pain may be selected from the group consisting of chronic joint pain, chronic osteoarthritic pain, chronic rheumatoid arthritic pain, chronic inflammatory pain, chronic headache, chronic menstrual pain, and chronic back pain. Chronic pain may be selected from the group consisting of chronic joint pain, chronic osteoarthritic pain, chronic rheumatoid arthritic pain, and chronic inflammatory pain. Chronic pain may be selected from the group consisting of chronic joint pain, chronic osteoarthritic pain, and chronic rheumatoid arthritic pain. Chronic pain may be selected from the group consisting of chronic joint pain and chronic osteoarthritic pain.

It should be appreciated that alleviating chronic pain means having an appreciable pain alleviating effect within 91, 60, 31, 28, 21, 14, 7, 3, or 2 days or 24, 12, 6, 3, 2, 1, 0.5, 0.25, 0.20. 0.17, or 0.10 hours after administering the first dose of active ingredient.

With respect to the assessment of a patients need for, or response to, treatment of the aforementioned pain states and abdominal aortic aneurysm pain, skin ulcer pain, or cancer pain, the physician may apply a pain assessment scale such as the Visual Analog Scale (“VAS”), wherein a patient is asked to indicate a point on a 100 millimeter line, having a left anchor of no pain and a right anchor of worst possible pain, corresponding to their degree of pain or the Likert score, wherein a patient is asked to categorize their pain or a numerical scale of from 0 (no pain) to 10 (worst possible pain).

In a clinical setting, a physician may assess a patients need for, or response to, treatment of osteoarthritis, rheumatoid arthritis, impaired joint function, pain, including osteoarthritic pain, rheumatoid arthritic pain, acute pain, joint pain, chronic pain, inflammatory pain, pain by administering a standard assessment questionnaire such as WOMAC or the Patient Global Impression of Change (“PGIC”).

Human patients in need of treatment with an invention compound may be identified by a medical practitioner using conventional means. For example, patients at risk of having asymptomatic joint cartilage damage (e.g., osteoarthritis patients) may be identified clinically by assaying synovial fluid from an asymptomatic, at-risk mammal for the presence of breakdown products from the extracellular matrix (for example, proteoglycans, type II cartilage, or hydroxyproline), specialized X-ray techniques, or nuclear magnetic resonance imaging (“MRI”) techniques. Human asymptomatic persons at-risk for cartilage damage or osteoarthritis include elite athletes, laborers such as foundry workers, bus drivers, or coal miners, persons with above-normal C-reactive protein levels, and persons with a family history of osteoarthritis. Further, persons presenting clinically with joint stiffness, joint pain, loss of joint function, or joint inflammation may be examined for joint cartilage damage using the above methods.

It should be appreciated that any invention method can be employed prophylactically to prevent or inhibit the onset of a disease or symptom thereof mediated by an MMP-13 enzyme. Patients who would benefit from prophylactic treatment include persons at risk for developing joint cartilage damage and persons who have developed joint cartilage damage but do not present clinically with secondary symptoms such as joint pain, joint stiffness, or in some cases, joint inflammation. These patients may be identified as described above.

The phrase “invention compound” means any compound of Formula I, or a pharmaceutically acceptable salt thereof, any crystal form thereof, or a pharmaceutically acceptable salt thereof, including solvates, stereoisomers, tautomers, etc. thereof, as defined herein.

The term “drugs”, which is synonymous with the phrases “active components”, “active compounds”, and “active ingredients”, includes any compound of Formula I, or a pharmaceutically acceptable salt thereof, any crystal form thereof, or a pharmaceutically acceptable salt thereof, as defined above, and may further include one or two of the other therapeutic agents described above.

The term “Thr245” means threonine 245 of an MMP-13 enzyme.

The term “Thr247” means threonine 247 of an MMP-13 enzyme.

The term “Met253” means methionine 253 of an MMP-13 enzyme.

The term “His251” means histidine 251 of an MMP-13 enzyme.

It should be appreciated that the matrix metalloproteinases include, but are not limited to, the following enzymes:
MMP-1, also known as interstitial collagenase, collagenase-1, or fibroblast-type collagenase;
MMP-2, also known as gelatinase A or 72 kDa Type IV collagenase;
MMP-3, also known as stromelysin or stromelysin-1;
MMP-7, also known as matrilysin or PUMP-1;
MMP-8, also known as collagenase-2, neutrophil collagenase or polymorphonuclear-type (“PMN-type”) collagenase;
MMP-9, also known as gelatinase B or 92 kDa Type IV collagenase;
MMP-10, also known as stromelysin-2;
MMP-11, also known as stromelysin-3;
MMP-12, also known as metalloelastase;
MMP-13, also known as collagenase-3;
MMP-14, also known as membrane-type (“MT”) 1-MMP or MT1-MMP;
MMP-15, also known as MT2-MMP;
MMP-16, also known as MT3-MMP;
MMP-17, also known as MT4-MMP;
MMP-18; and
MMP-19.

Other known MMPs include MMP-26 (Matrilysin-2).

The term “NSAID” is an acronym for the phrase “nonsteroidal anti-inflammatory drug”, which means any compound that inhibits cyclooxygenase-1 (“COX-1”) and cyclooxygenase-2. Most NSAIDs fall within one of the following five structural classes: (1) propionic acid derivatives, such as ibuprofen, naproxen, naprosyn, diclofenac, and ketoprofen; (2) acetic acid derivatives, such as tolmetin and sulindac; (3) fenamic acid derivatives, such as mefenamic acid and meclofenamic acid; (4) biphenylcarboxylic acid derivatives, such as diflunisal and flufenisal; and (5) oxicams, such as piroxim, peroxicam, sudoxicam, and isoxicam. Other useful NSAIDs include aspirin, acetominophen, indomethacin, and phenylbutazone. Selective inhibitors of cyclooxygenase-2 as described above may be considered to be NSAIDs also.

The phrases “effective amount” and “therapeutically effective amount” are synonymous and mean a sufficiently nontoxic amount of a compound of the present invention, a pharmaceutically acceptable salt thereof, or a solvate thereof, sufficient to effect an improvement of the condition (i.e., at least improvement of any single related pathology, sign, or symptom) being treated when administered to a mammal suffering from a disease that is mediated by MMP-13, or predicted to suffer from said disease in the future. A sufficiently nontoxic, therapeutically effective amount is an amount that does not cause a degree of toxicity in the target population that would be unacceptable to a drug regulatory authority such as the United States Food and Drug Administration (“FDA”), or equivalent foreign agency. For example in a human or other mammal, therapeutically effective amount can be determined experimentally in a laboratory or clinical setting, or it may be the amount required by the guidelines of the FDA for the particular mammal or mammalian population being treated. For example, the term “nontoxic” means the efficacious dose is 10 times or greater than the dose at which a toxic effect is observed in 10% or more of a patient population.

Other aspects of the present invention is an invention compound that is ≧10, ≧20, ≧50, ≧100, or ≧1000 times more potent versus MMP-13 than versus at least two of any other MMP enzyme or TACE. Still other aspects of the present invention are compounds of Formula I, or a pharmaceutically acceptable salt thereof, that are selective inhibitors of MMP-13 versus 2, 3, 4, 5, 6, or 7 other MMP enzymes, or versus TACE and 1, 2, 3, 4, 5, 6, or 7 other MMP enzymes.

Another aspect of the present invention is an invention compound that is selective inhibitors of MMP-13 versus MMP-1 or MMP-14. Still another aspect of this invention is an invention compound that is ≧10X more potent in vitro versus human MMP-13 full-length or catalytic domain than versus at least 5 other matrix metalloproteinase enzyme selected from the group consisting of human MMP-1 full-length, human MMP-2 full-length, human MMP-3 catalytic domain, human MMP-7 full-length, human MMP-8 full-length, human MMP-9 full-length, human MMP-12 catalytic domain, human MMP-14 catalytic domain, and human MMP-17 catalytic domain.

It should be appreciated that selectivity of an invention compound is a multidimensional characteristic that includes the number of other MMP enzymes and TACE over which selectivity for MMP-13 inhibition is present and the degree of selectivity of inhibition of MMP-13 over another particular MMP or TACE, as measured by, for example, the IC₅₀ in micromolar concentration of the compound for the inhibition of the other MMP enzyme or TACE divided by the IC₅₀ in micromolar concentration of the compound for the inhibition of MMP-13.

The invention provides a compound of Formula I, or a pharmaceutically acceptable salt thereof, which has an IC₅₀ with human MMP-13 catalytic domain that is less than or equal to 50 micromolar. Another aspect of this invention is a compound of Formula I, or a pharmaceutically acceptable salt thereof, which has an IC₅₀ with human MMP-13 catalytic domain that is less than or equal 10 micromolar, 1 micromolar, or 100 nanomolar.

It should be appreciated that until recently, the S1′ site of MMP-13 was previously thought to be a grossly linear channel which contained an opening at the top that allowed an amino acid side chain from a substrate molecule to enter during binding, and was closed at the bottom. The S1′ site is actually composed of an S1′ channel angularly connected to a newly discovered pocket which applicant calls the S1″ site. The S1″ site is open to solvent at the bottom, which can expose a functional group of an invention compound to solvent. For illustrative purposes, the S1′ site of the MMP-13 enzyme can now be thought of as being like a sock with a hole in the toes, wherein the S1′ channel is the region from approximately the opening to the ankle, and the S1″ site is the foot region below the ankle, which foot region is angularly connected to the ankle region.

More particularly, the S1′ channel is a specific part of the S1′ site and is formed largely by Leu218, Val219, His222 and by residues from Leu239 to Tyr244. The S1″ binding site is defined by residues from Tyr246 to Pro255. The S1″ site contains at least two hydrogen bond donors and aromatic groups which may interact with an invention compound.

Without wishing to be bound by any particular theory, the S1″ site could be a recognition site for triple helix collagen, the natural substrate for MMP-13. It is possible that the conformation of the S1″ site is modified only when an appropriate compound binds to MMP-13, thereby interfering with the collagen recognition process. This pattern of binding offers the possibility of greater selectivity than what is achievable with the binding pattern of known selective inhibitors of MMP-13, wherein the known binding pattern requires ligation of the catalytic zinc atom at the active site and occupation the S1′ channel, but not the S1″ site.

It should be appreciated that many invention compounds are amphoteric, and are thus capable of further forming pharmaceutically acceptable salts, including, but not limited to, acid addition and base addition salts. All pharmaceutically acceptable salt forms of the invention compounds are included within the scope of the present invention.

Pharmaceutically acceptable acid addition salts of an invention compound include salts derived from inorganic acids such as hydrochloric, nitric, phosphoric, sulfuric, hydrobromic, hydroiodic, hydrofluoric, phosphorous, and the like, as well salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like. Also contemplated are salts of amino acids such as arginate and the like and gluconate, galacturonate (see, for example, Berge S. M. et al., “Pharmaceutical Salts,” J. of Pharma. Sci., 1977;66: 1).

An acid addition salt of an invention compound is prepared by contacting the free base form of the compound with a sufficient amount of a desired acid to produce the salt in a conventional manner. The acid addition salt may be converted back to the free base form of the invention compound by contacting the acid addition salt with a base, and isolating the free base form of the compound in a conventional manner. The free base forms of the invention compounds differ from their respective acid addition salt forms somewhat in certain physical properties such as solubility, dissolution rate, crystal structure, hygroscopicity, and the like, but otherwise the free base forms of the compounds and their respective acid addition salt forms are equivalent for purposes of the present invention.

A pharmaceutically acceptable base addition salt of an invention compound may be prepared by contacting the free acid form of the compound with a sufficient amount of a desired base containing a metal cation such as an alkali or alkaline earth metal cation, or with an amine, especially an organic amine, to produce the salt in the conventional manner. Examples of suitable metal cations include sodium cation (Na⁺), potassium cation (K⁺), magnesium cation (Mg²⁺), calcium cation (Ca²⁺), and the like. Examples of suitable amines are N,N′-dibenzylethylenediamine, chloroprocaine, choline, diethanethanolamine, dicyclohexylamine, ethylenediamine, N-methylglucamine, and procaine (see, for example, Berge, supra., 1977).

A base addition salt of an invention compound may be converted back to the free acid form of the compound by contacting the base addition salt with an acid, and isolating the free acid of the invention compound in a conventional manner. The free acid forms of the invention compounds differ from their respective base addition salt forms somewhat in certain physical properties such as solubility, dissolution rate, crystal structure, hygroscopicity, and the like, but otherwise the base addition salts are equivalent to their respective free acid forms for purposes of the present invention.

The invention compounds can exist in unsolvated forms as well as solvated forms, including hydrated forms. In general, solvated forms, including hydrated forms, are equivalent to unsolvated forms and are included within the scope of the present invention. The present invention includes any unsolvated or solvated form of an invention compound.

Certain invention compounds can exist as amorphous solids. All amorphous solid forms of invention compounds are encompassed within the scope of the present invention.

Certain invention compounds can exist as crystalline solids. Each invention compound capable of existing as a crystalline solid may crystallize in one or more polymorphic forms depending on the conditions used for crystallization or storage. All polymorphic forms of crystalline invention compounds are encompassed within the scope of the present invention.

Some invention compounds possess chiral centers, and each center may exist in the (R) or (S) configuration. The present invention includes any stereoisomer of a compound of Formula I, or a pharmaceutically acceptable salt thereof, including any diastereomeric, enantiomeric, or epimeric form of the invention compounds, as well as mixtures thereof.

Some compounds of the present invention have alkenyl groups, which may exist as entgegen or zusammen conformations, in which case all geometric forms thereof, both entgegen (E) and zusammen (Z), cis and trans, and mixtures thereof, are within the scope of the present invention.

Some compounds of the present invention have cycloalkyl groups, which may be substituted at more than one carbon atom, in which case all geometric forms thereof, both cis and trans, and mixtures thereof, are within the scope of the present invention.

Certain invention compounds can exist as two or more tautomeric forms. Tautomeric forms of the invention compounds are forms that may interchange by shifting of the position of a hydrogen atom and a bond(s), for example, via enolization/de-enolization, 1,2-hydride, 1,3-hydride, or 1,4-hydride shifts, and the like. Tautomeric forms of an invention compound are isomeric forms of the invention compound that exist in a state of equilibrium, wherein the isomeric forms of the invention compound have the ability to interconvert by isomerization in situ, including in a reaction mixture, in an in vitro biological assay, or in vivo. An example of tautomeric forms is a 5-membered heteroaryl that is 1H- or 2H-tetrazol-5-yl. An invention compound includes any tautomeric form of the compound, as well as mixtures thereof.

The invention compounds also include isotopically-labelled compounds, which are identical to those recited above, but for the fact that one or more atoms are replaced by an atom having an atomic mass or mass number different from the atomic mass or mass number usually found in nature. Examples of isotopes that can be incorporated into the invention compounds include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. The invention compounds and their pharmaceutically acceptable salts that contain the aforementioned isotopes and/or other isotopes of other atoms are within the scope of this invention.

Certain isotopically labelled invention compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution of atoms in invention compounds with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of those described above in this invention can generally be prepared by art recognized procedures, or by carrying out the procedures incorporated by reference below, or procedures disclosed in the Schemes and/or in the Examples and Preparations, if any, below, by substituting a readily available isotopically labelled reagent for a non-isotopically labelled reagent.

As related above, the compounds of the present invention may be combined with other therapeutic agents for the treatment of certain diseases. For example for the treatment of rheumatoid arthritis, the compounds of the present invention may be combined with agents such as TNF-α inhibitors such as (i) anti-TNF monoclonal antibodies such as adalimumab, which is known in the United States by the trade name HUMIRA® and infliximab, which is marketed in the United States under the trade name REMICADE® for the treatment of moderately to severely active Crohn's disease for reduction of signs and symptoms in patients who do not adequately respond to conventional therapies and treatment of patients with fistulizing Crohn's disease for the reduction in the number of draining enterocutaneous fistula(s); (ii) TNF receptor immunoglobulin molecules such as etanercept, which is marketed in the United States under the trade name Enbrel® for the treatment of rheumatoid arthritis, juvenile rheumatoid arthritis, and psoriatic arthritis; (iii) low dose methotrexate; (iv) lefunimide, (v) hydroxychloroquine; (vi) d-penicillamine; (vii) auranofin; (viii) or parenteral or oral gold.

The compounds of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as those recited below, and intraarticular therapies such as corticosteroids and hyaluronic acids such as hyalgan and synvisc.

As mentioned above, the invention compounds can also be used in combination with existing therapeutic agents for the prevention or treatment of arthritis, including osteoarthritis, joint inflammation, and joint pain. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, selective COX-2 inhibitors such as those recited below, and the like, analgesics and intraarticular therapies such as corticosteroids and hyaluronic acids such as hyalgan and synvisc.

This invention also relates to a method of or a pharmaceutical composition for inhibiting joint cartilage damage and treating inflammatory processes and diseases comprising administering an invention compound to a mammal, including a human, cat, livestock or dog, wherein said joint cartilage damage and inflammatory processes and diseases are defined as above and said inhibitory compound is used in combination with one or more other therapeutically active agents under the following conditions:
A.) where a joint has become seriously inflamed as well as infected at the same time by bacteria, fungi, protozoa and/or virus, said inhibitory combination is administered in combination with one or more antibiotic, antifungal, antiprotozoal and/or antiviral therapeutic agents;
B.) where a multi-fold treatment of pain and inflammation is desired, said inhibitory combination is administered in combination with inhibitors of other mediators of inflammation, comprising one or more members independently selected from the group consisting essentially of:
(1) NSAIDs;
(2) H₁-receptor antagonists;
(3) kinin-B₁- and B₂-receptor antagonists;
(4) prostaglandin inhibitors selected from the group consisting of PGD-, PGF- PGI₂- and PGE-receptor antagonists;
(5) thromboxane A₂ (TXA₂-) inhibitors;
(6) 5-, 12- and 15-lipoxygenase inhibitors;
(7) leukotriene LTC₄-, LTD₄/LTE₄- and LTB₄-inhibitors;
(8) PAF-receptor antagonists;
(9) gold in the form of an aurothio group together with one or more hydrophilic groups;
(10) immunosuppressive agents selected from the group consisting of cyclosporine, azathioprine and methotrexate;
(11) anti-inflammatory glucocorticoids;
(12) penicillamine;
(13) hydroxychloroquine;
(14) anti-gout agents including colchicine; xanthine oxidase inhibitors including allopurinol; and uricosuric agents selected from the group consisting of probenecid, sulfinpyrazone and benzbromarone;
C. where older mammals are being treated for disease conditions, syndromes and symptoms found in geriatric mammals, said inhibitory combination is administered in combination with one or more members independently selected from the group consisting essentially of:
(1) cognitive therapeutics to counteract memory loss and impairment;
(2) anti-hypertensives and other cardiovascular drugs intended to offset the consequences of atherosclerosis, hypertension, myocardial ischemia, angina, congestive heart failure and myocardial infarction, selected from the group consisting of:
a. diuretics;
b. vasodilators;
c. β-adrenergic receptor antagonists;
d. angiotensin-II converting enzyme inhibitors (ACE-inhibitors), alone or optionally together with neutral endopeptidase inhibitors;
e. angiotensin II receptor antagonists;
f. renin inhibitors;
g. calcium channel blockers;
h. sympatholytic agents;
i. α₂-adrenergic agonists;
j. α-adrenergic receptor antagonists; and
k. HMG-CoA-reductase inhibitors (anti-hypercholesterolemics);
(3) antineoplastic agents selected from the group consisting of:
a. antimitotic drugs selected from the group consisting of:
i. vinca alkaloids selected from the group consisting of:
[1] vinblastine and
[2] vincristine;
(4) growth hormone secretagogues;
(5) strong analgesics;
(6) local and systemic anesthetics; and
(7) H₂-receptor antagonists, proton pump inhibitors and other gastroprotective agents.

The invention compounds may be administered in combination with inhibitors of other mediators of inflammation, comprising one or more members selected from the group consisting essentially of the classes of such inhibitors and examples thereof which include, matrix metalloproteinase inhibitors, aggrecanase inhibitors, TACE inhibitors, leukotriene receptor antagonists, IL-1 processing and release inhibitors, ILra, H₁-receptor antagonists; kinin-B₁- and B₂-receptor antagonists; prostaglandin inhibitors such as PGD-, PGF- PGI₂- and PGE-receptor antagonists; thromboxane A₂ (TXA2-) inhibitors; 5- and 12-lipoxygenase inhibitors; leukotriene LTC₄-, LTD₄/LTE₄- and LTB₄-inhibitors; PAF-receptor antagonists; MEK inhibitors; IKK inhibitors; MKK inhibitors; gold in the form of an aurothio group together with various hydrophilic groups; immunosuppressive agents, e.g., cyclosporine, azathioprine and methotrexate; anti-inflammatory glucocorticoids; penicillamine; hydroxychloroquine; anti-gout agents, e.g., colchicine, xanthine oxidase inhibitors, e.g., allopurinol and uricosuric agents, e.g., probenecid, sulfinpyrazone and benzbromarone.

The invention compounds may also be used in combination with anticancer agents such as endostatin and angiostatin or cytotoxic drugs such as adriamycin, daunomycin, cis-platinum, etoposide, taxol, taxotere and alkaloids, such as vincristine and antimetabolites such as methotrexate.

The invention compounds may also be used in combination with anti-hypertensives and other cardiovascular drugs intended to offset the consequences of atherosclerosis, including hypertension, myocardial ischemia including angina, congestive heart failure and myocardial infarction, selected from the group consisting of vasodilators such as hydralazine, β-adrenergic receptor antagonists such as propranolol, calcium channel blockers such as nifedipine, α₂-adrenergic agonists such as clonidine, α-adrenergic receptor antagonists such as prazosin and HMG-CoA-reductase inhibitors (anti-hypercholesterolemics) such as lovastatin or atorvastatin.

The invention compounds may also be administered in combination with one or more antibiotic, antifungal, antiprotozoal, antiviral or similar therapeutic agents.

The invention compounds may also be used in combination with CNS agents such as antidepressants (such as sertraline), anti-Parkinsonian drugs (such as L-dopa, requip, mirapex, MAOB inhibitors such as selegine and rasagiline, comP inhibitors such as Tasmar, A-2 inhibitors, dopamine reuptake inhibitors, NMDA antagonists, nicotine agonists, dopamine agonists and inhibitors of neuronal nitric oxide synthase) and anti-Alzheimer's drugs such as donepezil, tacrine, COX-2 inhibitors, propentofylline or metryfonate.

The invention compounds may also be used in combination with osteoporosis agents such as roloxifene, lasofoxifene, droloxifene or fosomax and immunosuppressant agents such as FK-506 and rapamycin.

Other mammalian diseases and disorders which are treatable by administration of an invention compound alone, an invention combination, or a pharmaceutical composition comprising the compound or combination as defined below, may include: rheumatic diseases such as arthritis, inflammatory skin diseases such as psoriasis, eczema, atopic dermatitis, discoid lupus, contact dermatitis, bullous pemphigoid, vulgaris, and alopecia areata, fever (including rheumatic fever and fever associated with influenza and other viral infections), fibromyalgia, sleep disorders, common cold, dysmenorrhea, menstrual cramps, inflammatory bowel disease, Crohn's disease, emphysema, acute respiratory distress syndrome, asthma, bronchitis, chronic obstructive pulmonary disease, Alzheimer's disease, organ transplant toxicity, cachexia, allergic reactions, allergic contact hypersensitivity, cancer (such as solid tumor cancer including colon cancer, breast cancer, lung cancer and prostrate cancer; hematopoietic malignancies including leukemias and lymphomas; Hodgkin's disease; aplastic anemia, skin cancer and familiar adenomatous polyposis), tissue ulceration, peptic ulcers, gastritis, regional enteritis, ulcerative colitis, diverticulitis, recurrent gastrointestinal lesion, gastrointestinal bleeding, coagulation, anemia, synovitis, gout, ankylosing spondylitis, restenosis, periodontal disease, epidermolysis bullosa, osteoporosis, loosening of artificial joint implants, atherosclerosis (including atherosclerotic plaque rupture), aortic aneurysm (including abdominal aortic aneurysm and brain aortic aneurysm), periarteritis nodosa, congestive heart failure, myocardial infarction, stroke, cerebral ischemia, head trauma, spinal cord injury, neuralgia, neuro-degenerative disorders (acute and chronic), autoimmune disorders, Huntington's disease, Parkinson's disease, migraine, depression, peripheral neuropathy, pain (including low back and neck pain, headache, toothache, and neuropathic pain), gingivitis, cerebral amyloid angiopathy, nootropic or cognition enhancement, amyotrophic lateral sclerosis, multiple sclerosis, ocular angiogenesis, corneal injury, macular degeneration, conjunctivitis, abnormal wound healing, muscle or joint sprains or strains, tendonitis, skin disorders (such as psoriasis, eczema, scleroderma and dermatitis), myasthenia gravis, polymyositis, myositis, bursitis, burns, diabetes (including types I and II diabetes, diabetic retinopathy, neuropathy and nephropathy), tumor invasion, tumor growth, tumor metastasis, corneal scarring, scleritis, immunodeficiency diseases (such as AIDS in humans and FLV, FIV in cats), sepsis, premature labor, hypoprothrombinemia, hemophilia, thyroiditis, sarcoidosis, Behcet's syndrome, hypersensitivity, kidney disease, Rickettsial infections (such as Lyme disease, Erlichiosis), Protozoan diseases (such as malaria, giardia, coccidia), reproductive disorders (preferably in livestock), epilepsy, convulsions, and septic shock.

All that is required to practice a method of this invention is to administer to a patient a compound of Formula I, or a pharmaceutically acceptable salt thereof, in a sufficiently nontoxic amount that is therapeutically effective for preventing, inhibiting, or reversing the condition being treated. The invention compound can be administered directly or as part of a pharmaceutical composition.

Determination of proper dosage forms, dosage amounts, and routes of administration for treatment or prophylactic administration is within the level of ordinary skill in the pharmacentical medical or veterinarian arts. In one aspect of this invention, a therapeutically effective amount, or, simply, effective amount, of an invention compound will generally be from about 1 to about 300 mg/kg of subject body weight of the compound of Formula I, or a pharmaceutically acceptable salt thereof. Typical doses will be from about 10 to about 5000 mg/day for an adult mammal of normal weight. In a clinical setting, regulatory agencies such as, for example, the Food and Drug Administration (“FDA”) in the U.S. may require a particular therapeutically effective amount.

In determining what constitutes a nontoxic effective amount or a therapeutically effective amount of an invention compound for treating, preventing, or reversing one or more symptoms of any one of the diseases and disorders described above that are being treated according to the invention methods, a number of factors will generally be considered by the medical practitioner or veterinarian in view of the experience of the medical practitioner or veterinarian, including the Food and Drug Administration guidelines, or guidelines from an equivalent agency, published clinical studies, the subject's (e.g., mammal's) age, sex, weight and general condition, as well as the type and extent of the disease, disorder or condition being treated, and the use of other medications, if any, by the subject. As such, the administered dose may fall within the ranges or concentrations recited above, or may vary outside them, ie, either below or above those ranges, depending upon the requirements of the individual subject, the severity of the condition being treated, and the particular therapeutic formulation being employed. Generally, treatment may be initiated using smaller dosages of the invention compound that are less than optimum for a particular subject. Thereafter, the dosage can be increased by small increments until the optimum effect under the circumstance is reached. For convenience, the total daily dosage may be divided and administered in portions during the day, if desired.

The present invention also relates to the formulation of a compound of the present invention alone or with one or more other therapeutic agents which are to form the intended combination, including wherein said different drugs have varying half-lives, by creating controlled-release forms of said drugs with different release times which achieves relatively uniform dosing; or, in the case of non-human patients, a medicated feed dosage form in which said drugs used in the combination are present together in admixture in the feed composition. There is further provided in accordance with the present invention co-administration in which the combination of drugs is achieved by the simultaneous administration of said drugs to be given in combination; including co-administration by means of different dosage forms and routes of administration; the use of combinations in accordance with different but regular and continuous dosing schedules whereby desired plasma levels of said drugs involved are maintained in the patient being treated, even though the individual drugs making up said combination are not being administered to said patient simultaneously.

Pharmaceutical compositions of an invention compound or combination may be produced by formulating the invention compound or combination in dosage unit form with a pharmaceutical carrier. Some examples of dosage unit forms are tablets, capsules, pills, powders, aqueous and nonaqueous oral solutions and suspensions, and parenteral solutions packaged in containers containing either one or some larger number of dosage units and capable of being subdivided into individual doses. Alternatively, the invention compounds may be formulated separately.

Some examples of suitable pharmaceutical carriers, including pharmaceutical diluents, are gelatin capsules; sugars such as lactose and sucrose; starches such as corn starch and potato starch; cellulose derivatives such as sodium carboxymethyl cellulose, ethyl cellulose, methyl cellulose, and cellulose acetate phthalate; gelatin; talc; stearic acid; magnesium stearate; vegetable oils such as peanut oil, cottonseed oil, sesame oil, olive oil, corn oil, and oil of theobroma; propylene glycol, glycerin; sorbitol; polyethylene glycol; water; agar; alginic acid; isotonic saline, and phosphate buffer solutions; as well as other compatible substances normally used in pharmaceutical formulations.

The compositions to be employed in the invention can also contain other components such as coloring agents, flavoring agents, and/or preservatives. These materials, if present, are usually used in relatively small amounts. The compositions can, if desired, also contain other therapeutic agents commonly employed to treat any of the above-listed diseases and disorders.

The percentage of the active ingredients of a compound of Formula I, or a pharmaceutically acceptable salt thereof, in the foregoing compositions can be varied within wide limits, but for practical purposes it is preferably present in a total concentration of at least 10% in a solid composition and at least 2% in a primary liquid composition. The most satisfactory compositions are those in which a much higher proportion of the active ingredients are present, for example, up to about 95%.

Preferred routes of administration of an invention compound are oral or parenteral. However, another route of administration may be preferred depending upon the condition being treated. For exampled, topical administration or administration by injection may be preferred for treating conditions localized to the skin or a joint. Administration by transdermal patch may be preferred where, for example, it is desirable to effect sustained dosing.

It should be appreciated that the different routes of administration may require different dosages. For example, a useful intravenous (“IV”) dose is between 5 and 50 mg, and a useful oral dosage is between 20 and 800 mg, of a compound of Formula I, or a pharmaceutically acceptable salt thereof. The dosage is within the dosing range used in treatment of the above-listed diseases, or as would be determined by the needs of the patient as described by the physician.

The invention compounds or combinations may be administered in any form. Preferably, administration is in unit dosage form. A unit dosage form of the invention compound to be used in this invention may also comprise other compounds useful in the therapy of diseases described above. A further description of pharmaceutical formulations useful for administering the invention compounds and invention combinations is provided below.

The active components of the invention combinations, may be formulated together or separately and may be administered together or separately. The particular formulation and administration regimens used may be tailored to the particular patient and condition being treated by a practitioner of ordinary skill in the medical or pharmaceutical arts.

Advantages:

The advantages of using an invention compound in a method of the instant invention include the nontoxic nature of the compounds at and substantially above therapeutically effective doses, their ease of preparation, the fact that the compounds are well-tolerated, and the ease of topical, IV, or oral administration of the drugs.

Another important advantage is the disease modifying properties of the invention compounds, which provide prevention or inhibition of underlying MMP-13 mediated disease pathologies such as cartilage degradation, penetration of the extracellular matrix in cancer metastasis or angiogenesis, and degradation of the extracellular collagens that impart strength and proper form to a heart muscle.

Preparations:

Compounds of this invention may be prepared using synthetic organic chemistry methodology well known to those skilled in the art of organic chemistry. Representative of compounds of this invention are outlined in the schemes and described in the examples below. In the description of the representative syntheses, the following definitions are used:
DCC means 1,3-dicyclohexylcarbodiimide
CDI means N,N′-carbonyldiimidazole
EDC, EDAC, and EDCI mean 1-(3-dimethylaminopropyl)-3-ethylcarbodiimide hydrochloride
BOC means tertiary-butyloxycarbonyl
CBZ means benzyloxycarbonyl
FMOC means fluorenylmethoxycarbonyl
BuLi means a butyl lithium selected from the group consisting of n-BuLi, sec-BuLi, and t-BuLi
n-BuLi means normal-butyl lithium
sec-BuLi means secondary-butyl lithium
t-BuLi means tertiary-butyl lithium
psi means pounds per square inch
Ra Ni means Raney nickel
THF means tetrahydrofuran
BOC₂O means di-(tertiary-butyl)dicarbonate
TMEDA means N,N, N′, N′-tetramethylenediamine
TFA means trifluoroacetic acid
Dppf-PdCl2 means 1,1′-bis(diphenylphosphino)ferrocene dichloropalladium(II), (1:1) complex with dichloromethane
Dppp-Pd(OAc)₂ means palladium acetate 1,3-bis(diphenylphosphino)propane, which may be prepared in situ by combining a solution of palladium acetate and 1,3-bis(diphenylphosphino)propane
Et₃N means triethylamine
DMF means N,N-dimethylformamide
HPLC means high performance liquid chromatography
pXRD means powder X-ray diffractometry
TGA means thermogravimetric analysis
DSC means differential scanning calorimetry

Intermediates for the synthesis of a compound of Formula I, or a pharmaceutically acceptable salt thereof, may be prepared by one of ordinary skill in the art of organic chemistry by adapting various synthetic procedures incorporated by reference above or that are well-known in the art of organic chemistry. These synthetic procedures may be found in the literature in, for example, Reagents for Organic Synthesis, by Fieser and Fieser, John Wiley & Sons, Inc, New York, 2000; Comprehensive Organic Transformations, by Richard C. Larock, VCH Publishers, Inc, New York, 1989; the series Compendium of Organic Synthetic Methods,1989,by Wiley-Interscience; the text Advanced Organic Chemistry, 4^{th} edition, by Jerry March, Wiley-Interscience, New York,1992; or the Handbook of Heterocyclic Chemistry by Alan R. Katritzky, Pergamon Press Ltd, London, 1985, to name a few. Alternatively, a skilled artisan may find methods useful for preparing the intermediates in the chemical literature by searching widely available databases such as, for example, those available from the Chemical Abstracts Service, Columbus, Ohio, or MDL Information Systems GmbH (formerly Beilstein Information Systems GmbH), Frankfurt, Germany.

Preparations of the invention compounds may use starting materials, reagents, solvents, and catalysts that may be purchased from commercial sources or they may be prepared by adapting procedures described or cited in the resources referenced above. Commercial sources of starting materials, reagents, solvents, and catalysts useful in preparing invention compounds include, for example, The Aldrich Chemical Company, and other subsidiaries of Sigma-Aldrich Corporation, St. Louis, Mo., BACHEM, BACHEM A. G., Switzerland, or Lancaster Synthesis Ltd, United Kingdom.

Syntheses of some invention compounds may utilize starting materials, intermediates, or in situ reaction products that contain at least one targeted functional group that is to be transformed by any given reaction step and one or more ancillary functional groups that must remain intact during that reaction step and, perhaps, subsequent reaction steps. However, during any given reaction step, a particular ancillary functional group may itself be vulnerable to side-reacting under the reaction step conditions, even to the extent of being more reactive to the reaction step conditions than is the targeted functional group.

During such chemical reaction steps, a reactive ancillary functional group may be protected from reacting by a protecting group that renders the reactive ancillary functional group substantially inert to the reaction conditions employed. A protecting group is introduced onto a starting material prior to carrying out the reaction step(s) for which a protecting group is needed. Once the reaction step(s) is carried out and the protecting group is no longer needed, the protecting group can be removed.

It is well within the ordinary skill in the art to introduce protecting groups during a synthesis of a compound of Formula I, or a pharmaceutically acceptable salt thereof, and then later remove them. Procedures for introducing and removing protecting groups are known and referenced such as, for example, in Protective Groups in Organic Synthesis, 2^{nd} ed., Greene T. W. and Wuts P. G., John Wiley & Sons, New York: N.Y., 1991, which is hereby incorporated by reference.

Thus, for example, protecting groups such as the following may be utilized to protect amino, hydroxyl, and other groups: carboxylic acyl groups such as, for example, formyl, acetyl, and trifluoroacetyl; alkoxycarbonyl groups such as, for example, ethoxycarbonyl, tert-butoxycarbonyl (BOC), β, β, β-trichloroethoxycarbonyl (TCEC), and β-iodoethoxycarbonyl; aralkyloxycarbonyl groups such as, for example, benzyloxycarbonyl (CBZ), para-methoxybenzyloxycarbonyl, and 9-fluorenylmethyloxycarbonyl (FMOC); trialkylsilyl groups such as, for example, trimethylsilyl (TMS) and tert-butyldimethylsilyl (TBDMS); and other groups such as, for example, triphenylmethyl (trityl), tetrahydropyranyl, vinyloxycarbonyl, ortho-nitrophenylsulfenyl, diphenylphosphinyl, para-toluenesulfonyl (Ts), mesyl, trifluoromethanesulfonyl, and benzyl. Examples of procedures for removal of protecting groups include hydrogenolysis of CBZ groups using, for example, hydrogen gas at 50 psi in the presence of a hydrogenation catalyst such as 10% palladium on carbon, acidolysis of BOC groups using, for example, hydrogen chloride in dichloromethane, trifluoroacetic acid (TFA) in dichloromethane, and the like, reaction of silyl groups with fluoride ions, and reductive cleavage of TCEC groups with zinc metal.

Some syntheses of compounds of the present invention described herein may employ protecting groups while others may not. In any event, syntheses of the compounds of Formula I that may be employed to make the compounds are illustrated below in Schemes 1 to 9 and the examples. In the schemes, it should be appreciated that R¹, L¹ , L², V, L³, and R² are as defined above for Formula I. Further in the schemes, it should be appreciated that one of ordinary skill in the organic chemistry art would know that an acid or base work-up may be required to produce the particular form of a reaction product illustrated therein. These work-ups may or may not be literally recited in the schemes. 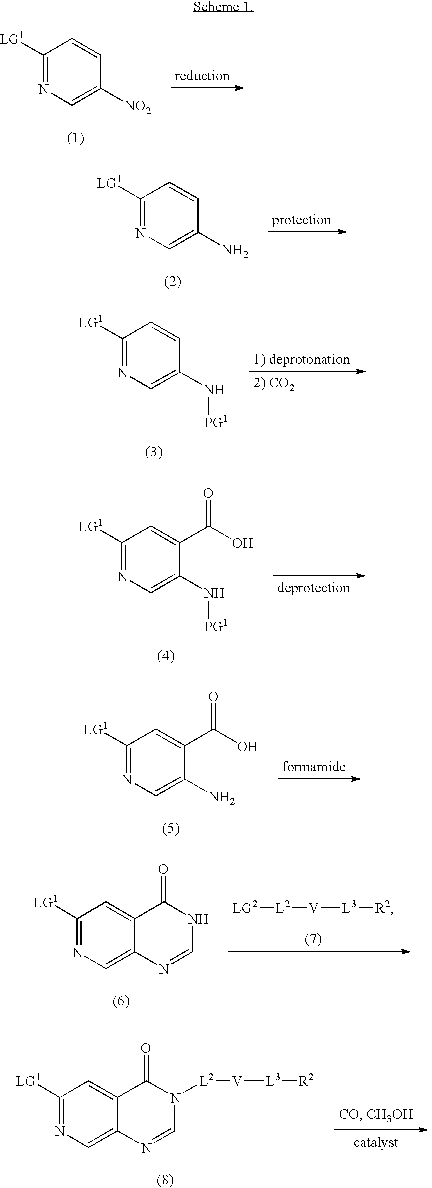 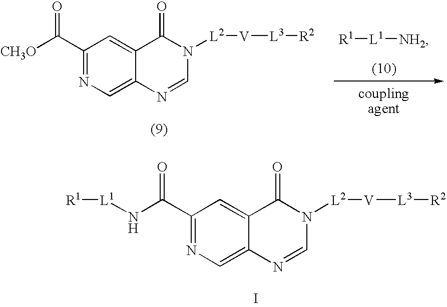
wherein LG¹ and LG² are leaving groups independently selected from the group consisting of Cl, Br, I, CF₃SO₃, and the like, or LG²-L²—are taken together to form an imine selected from the group consisting of CH₂═N— or CH(R^{X})═N—, and PG¹ is an amine protecting group selected from the group consisting of BOC, CBZ, FMOC, and the like. 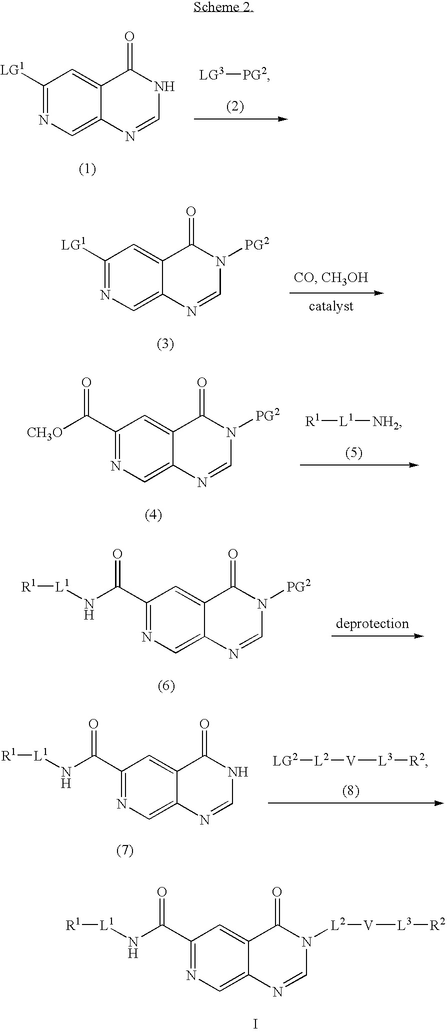
wherein the compound of formula (1) may be prepared as illustrated above in Scheme 1, LG¹, LG², and LG³ are leaving groups independently selected from the group consisting of Cl, Br, I, CF₃SO₃, and the like, or LG²-L^{8 2}—are taken together to form an imine selected from the group consisting of CH₂═N— or CH(R^{X})═N—, and PG² is an amide protecting group selected from the group consisting of benzyl, 4-methoxybenzyl, trityl, 2-chloroethyl, 2-trimethylsilyl-ethyl, and the like. 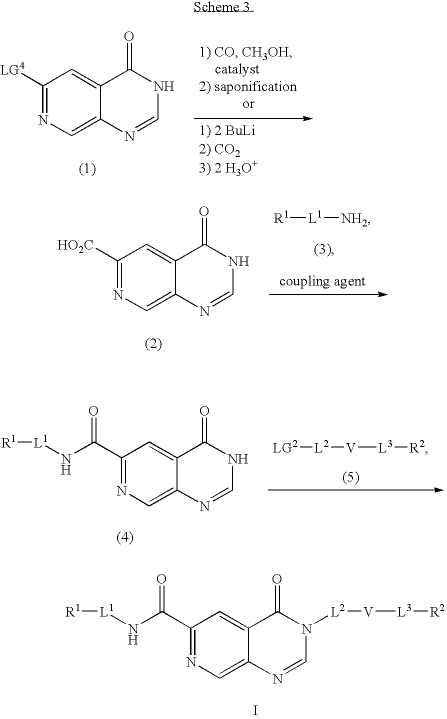
wherein the compound of formula (1) may be prepared by a procedure analogous to the preparation of the compound of formula (6) illustrated above in Scheme 1, LG⁴ is a leaving group selected from the group consisting of Cl, Br, and I, LG² is a leaving group independently selected from the group consisting of Cl, Br, I, CF₃SO₃, and the like, or LG²-L2—are taken together to form an imine selected from the group consisting of CH₂═N— or CH(R^{X})═N—, and coupling agent is useful for coupling an amine with a carboxylic acid such as those selected from the group consisting of DCC, CDI, EDC, and the like. 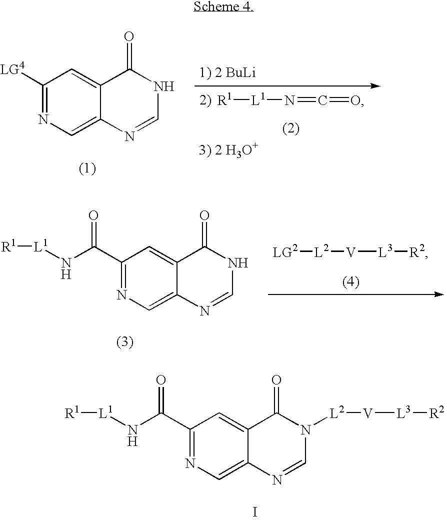
wherein the compound of formula (1) may be prepared as illustrated above in Scheme 3, LG⁴ is a leaving group selected from the group consisting of Cl, Br, and I, LG² is a leaving group independently selected from the group consisting of Cl, Br, I, CF₃SO₃, and the like or LG²-L²—are taken together to form an imine selected from the group consisting of CH₂═N— or CH(R^{X})═N—. 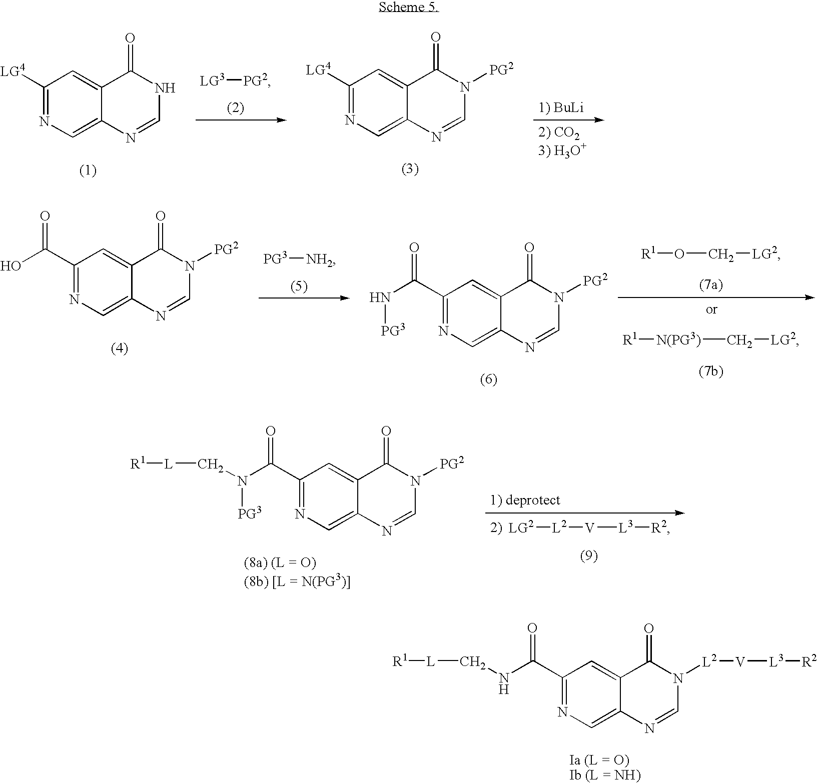
wherein the compound of formula (1) may be prepared as illustrated above in Scheme 3, LG⁴ is a leaving group selected from the group consisting of Cl, Br, and I, each LG² is independently a leaving group independently selected from the group consisting of Cl, Br, I, CF₃SO₃, and the like, LG³ is a leaving group independently selected from the group consisting of Cl, Br, I, CF₃SO₃, and the like, PG² is an amide protecting group selected from the group consisting of benzyl, 4-methoxybenzyl, trityl, 2-chloroethyl, 2-trimethylsilyl-ethyl, and the like, and each PG³ is independently an amine protecting group selected from the group consisting of benzyl, 4-methoxybenzyl, trityl, 2-chloroethyl, 2-trimethylsilyl-ethyl, and the like. 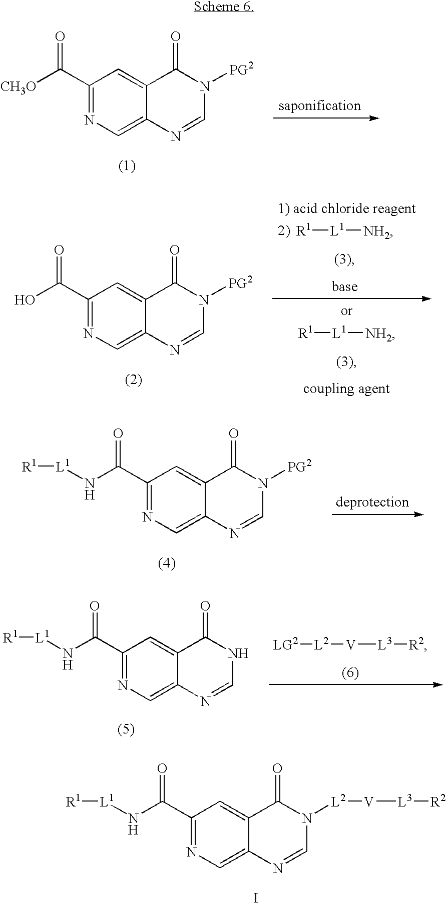
wherein the compound of formula (1) may be prepared as illustrated above in Scheme 2 for the preparation of a compound of formula (4), acid chloride reagent is useful for generating a carboxylic acid chloride from the corresponding carboxylic acid such as those selected from the group consisting of thionyl chloride, oxalyl chloride, and the like, base means a non-nucleophilic base such as Et₃N, K₂CO₃, NaH, and the like that is capable of deprotonating, at least in part, a protonated primary amine, coupling agent is useful for coupling an amine with a carboxylic acid such as those selected from the group consisting of DCC, CDI, EDC, and the like, and PG² is an amide protecting group selected from the group consisting of benzyl, 4-methoxybenzyl, trityl, 2-chloroethyl, 2-trimethylsilyl-ethyl, and the like. 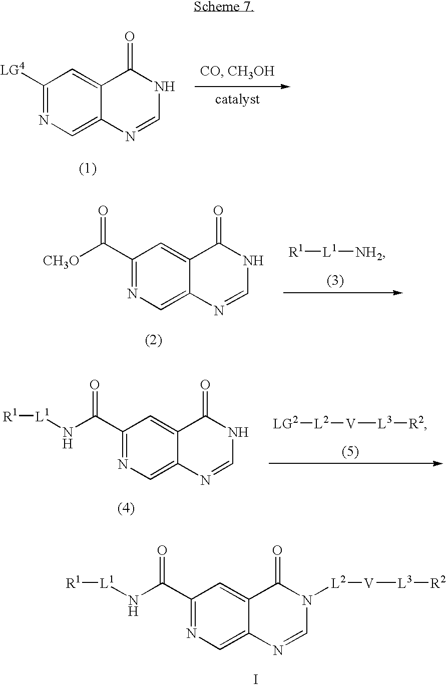
wherein the compound of formula (1) may be prepared as illustrated above in Scheme 3, LG⁴ is a leaving group selected from the group consisting of Cl, Br, and I, LG² is a leaving group independently selected from the group consisting of Cl, Br, I, CF₃SO₃, and the like or LG²-L²—are taken together to form an imine selected from the group consisting of CH₂═N— or CH(R^{X})═N—. 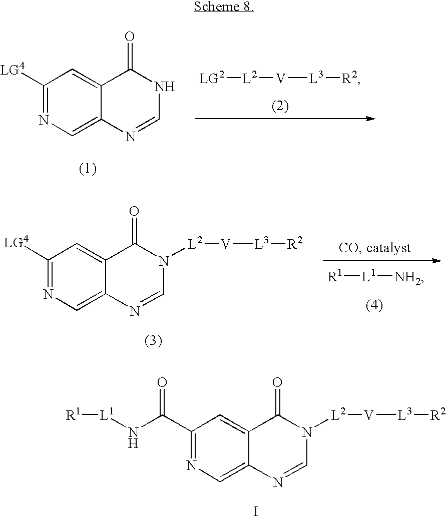
wherein the compound of formula (1) may be prepared as illustrated above in Scheme 3, LG⁴ is a leaving group selected from the group consisting of Cl, Br, and I, LG² is a leaving group independently selected from the group consisting of Cl, Br, I, CF₃SO₃, and the like or LG²-L²—are taken together to form an imine selected from the group consisting of CH₂═N— or CH(R^{X})═N—. 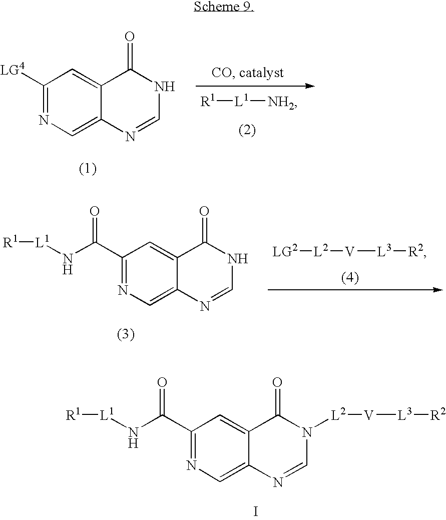
wherein the compound of formula (1) may be prepared as illustrated above in Scheme 3, LG⁴ is a leaving group selected from the group consisting of Cl, Br, and I, LG² is a leaving group independently selected from the group consisting of Cl, Br, I, CF₃SO₃, and the like or LG²-L²—are taken together to form an imine selected from the group consisting of CH₂═N— or CH(R^{X})═N—.

In Scheme 1, a substituted 3-nitro-pyridine of formula (1) is reduced using catalytic conditions such as hydrogenation at hydrogen gas pressure of from about 15 psi to more than 100 psi over a suitable catalyst such as Ra Ni, or hydrazine with 5-20% palladium on carbon catalyst, in a solvent such as THF at a temperature from about room temperature to more than 100° C. to give a substituted 3-amino-pyridine of formula (2). Alternatively, a chemical reduction of compound of formula (1) to compound of formula (2) using, for example, zinc with hydrochloric acid, or sodium borohydride with titanium tetrachloride may be employed. The substituted 3-amino-pyridine of formula (2) is then protected with an amide protecting group selected from the group consisting of BOC, CBZ, FMOC, and the like by reaction with, for example, BOC₂O in a solvent such as dioxane or THF at a temperature from about room temperature to more than 120° C. to give a substituted 3-(protected amino)-pyridine of formula (3). The substituted 3-(protected amino)-pyridine of formula (3) is then deprotonated with a strong base such as n-BuLi, optionally in the presence of a ligand such as TMEDA, at temperatures from about −80° C. to more than room temperature, followed by quenching of the resulting carbanion formed in situ thereby, wherein the quenching is carried by addition of anhydrous carbon dioxide gas, or alternatively by adding the carbanion solution to crushed dry ice, to give a substituted 3-(protected amino)-pyridine-4-carboxylic acid of formula (4). The amino group in the substituted 3-(protected amino)-pyridine-4-carboxylic acid of formula (4) is then deprotected, such as by treatment with TFA in CH₂CL₂ when the protecting group is a BOC group, or by catalytic hydrogenolysis when the protecting group is a CBZ or FMOC, at temperatures from about 0° C. to more than room temperature to give a substituted 3-amino-pyridine-4-carboxylic acid of formula (5). The substituted 3-amino-pyridine-4-carboxylic acid of formula (5) is then condensed with formamide under cyclizing conditions to give a substituted pyrido[3,4-d]pyrimidin-4-one of formula (6). The substituted pyrido[3,4-d]pyrimidin-4-one of formula (6) is then coupled with the compound of formula (7) to give a substituted pyrido[3,4-d]pyrimidin-4-one of formula (8). The substituted pyrido[3,4-d]pyrimidin-4-one of formula (8) is then coupled with CO at a pressure of from about 50 psi to more than 1000 psi in methanol in the presence of a suitable catalyst such as dppf-PdCl2 or Dppp-Pd(OAc)₂ with a suitable aprotic base such as Et₃N at suitable temperatures of from about room temperature to more than 200° C. to give a methyl ester of formula (9). The methyl ester of formula (9) is then condensed with an amine of formula (10) in the presence of a suitable coupling agent such as (CH₃)₃Al in a suitable solvent such as toluene and/or THF at a temperature from about 0° C. to about 100° C. to give the compound of Formula I as described above.

In Scheme 2, a substituted pyrido[3,4-d]pyrimidin-4-one of formula (1), which corresponds to the substituted pyrido[3,4-d]pyrimidin-4-one of formula (6) from Scheme 1, is protected with a suitable amide protecting group of formula (2) to give a 3-protected substituted pyrido[3,4-d]pyrimidin-4-one of formula (3). The 3-protected substituted pyrido[3,4-d]pyrimidin-4-one of formula (3) is then converted to the compound of Formula I as defined above according to the corresponding procedures described above for Scheme 1 and a deprotection step that may comprise catalytic hydrogenolysis as described above for Scheme 1 or detritylation with a suitable nucleophile such as Na₂S in an alcohol.

In Scheme 3, a substituted pyrido[3,4-d]pyrimidin-4-one of formula (1), which is prepared in a manner analogous to that described in Scheme 1 for the preparation of the substituted pyrido[3,4-d]pyrimidin-4-one of formula (6), is coupled with CO at a pressure of from about 50 psi to more than 1000 psi in methanol in the presence of a suitable catalyst such as dppf-PdCl₂ or Dppp-Pd(OAc)₂ with a suitable aprotic base such as Et₃N at suitable temperatures of from about room temperature to more than 200° C. to give a methyl ester, which is saponified to a corresponding substituted 4-oxo-pyrido[3,4-d]pyrimidin-6-carboxylic acid of formula (2). Alternatively, the substituted pyrido[3,4-d]pyrimidin-4-one of formula (1) is twice deprotonated with a strong base such as n-BuLi, optionally in the presence of a ligand such as TMEDA, at temperatures from about −80° C. to more than room temperature, followed by quenching of the resulting dianion formed in situ thereby, wherein the quenching is carried out by addition of anhydrous carbon dioxide gas, or alternatively adding the carbanion solution to crushed dry ice, to give the substituted 4-oxo-pyrido[3,4-d]pyrimidin-6-carboxylic acid of formula (2). The substituted 4-oxo-pyrido[3,4-d]pyrimidin-6-carboxylic acid of formula (2) is then coupled with an amine of formula (3) in the presence of a coupling agent such as DCC, CDI, EDC, and the like in an aprotic solvent such as THF, dioxane, CH₂Cl₂, and the like at temperatures from about 0° C. to more than 100° C. to give the amide of formula (4). The amide of formula (4) is then coupled with a compound of formula (5) to give a compound of Formula I as defined above according to the corresponding procedure described above for Scheme 1.

In Scheme 4, a substituted pyrido[3,4-d]pyrimidin-4-one of formula (1), which is prepared in a manner analogous to that described in Scheme 1 for the preparation of the substituted pyrido[3,4-d]pyrimidin-4-one of formula (6), is twice deprotonated with a strong base such as n-BuLi, optionally in the presence of a ligand such as TMEDA, at temperatures from about −80° C. to more than room temperature, followed by quenching of the resulting dianion formed in situ thereby, wherein the quenching is carried out by addition of an isocyanate of formula (2), prepared by conventional means by reaction of a corresponding amine of formula R¹-L¹-NH₂ with a reagent such as phosgene, triphosgene, and the like, to give the amide of formula (3). The amide of formula (3) is then coupled with a compound of formula (4) to give a compound of Formula I as defined above according to the corresponding procedure described above for Scheme 1.

In Scheme 5, a substituted pyrido[3,4-d]pyrimidin-4-one of formula (1), which is prepared in a manner analogous to that described in Scheme 1 for the preparation of the substituted pyrido[3,4-d]pyrimidin-4-one of formula (6), is protected with a compound of formula (2) to give a protected substituted pyrido[3,4-d]pyrimidin-4-one of formula (3). The protected substituted pyrido[3,4-d]pyrimidin-4-one of formula (3) is then allowed to undergo a lithium-halogen exchange reaction by contact with BuLi, at temperatures from about −80° C. to more than room temperature, followed by quenching of the resulting carbanion formed in situ thereby, wherein the quenching is carried by addition of anhydrous carbon dioxide gas, or alternatively adding the carbanion solution to crushed dry ice, to give a protected substituted 4-oxo-pyrido[3,4-d]pyrimidin-6-carboxylic acid of formula (4). The protected substituted 4-oxo-pyrido[3,4-d]pyrimidin-6-carboxylic acid of formula (4) is then coupled with a protected amine of formula (5) as described above for the corresponding reaction in Scheme 3 to give the protected substituted 4-oxo-pyrido[3,4-d]pyrimidin-6-carboxylic amide of formula (6). The protected substituted 4-oxo-pyrido[3,4-d]pyrimidin-6-carboxylic amide of formula (6) is then coupled with an ether of formula (7a) or a protected amine of formula (7b) to give a protected substituted 4-oxo-pyrido[3,4-d]pyrimidin-6-carboxylic amide of formula (8a) or (8b), respectively. The protected substituted 4-oxo-pyrido[3,4-d]pyrimidin-6-carboxylic amide of formula (8a) or (8b) is then per deprotected as described above for the corresponding reactions in Schemes 1 and 2, and the resulting per deprotected lactam is then coupled with a compound of formula (9) to give a compound of Formula Ia or Ib, respectively, which are compounds of Formula I wherein L¹ is an O—CH₂ or N(H)—CH₂ diradical, according to the corresponding procedure described above for Scheme 1. Alternatively, the PG² protecting group is selectively removed without removing the PG³ protecting group, and the resulting selectively deprotected lactam is coupled with a compound of formula (9), and then the remaining protecting groups PG³ are removed to give the compound of Formula Ia or Ib, respectively.

In Scheme 6, a protected carboxylic ester of formula (1), which is prepared in Scheme 2, is saponified under conventional basic or acidic conditions to give the corresponding protected carboxylic acid of formula (2). The protected carboxylic acid of formula (2) is then allowed to react with an acid chloride reagent such as a reagent selected from the group consisting of thionyl chloride and oxalyl chloride to give a corresponding protected carboxylic acid chloride in situ, which is then coupled with an amine of formula (3) in the presence of an aprotic base such as Et₃N or K₂CO₃ to give a protected carboxylic amide of formula (4). Alternatively, the protected carboxylic acid of formula (2) is coupled with the amine of formula (3) in the presence of a coupling agent such as DCC, CDI, EDC, and the like in an aprotic solvent such as THF, dioxane, CH₂Cl₂, and the like at temperatures from about 0° C. to more than 100° C. to give the amide of formula (4). The protected carboxylic amide of formula (4) is then deprotected and the resulting deprotected carboxylic amide is coupled with a compound of formula (6) as described for Scheme 2 to give a compound of Formula I as defined above.

In Scheme 7, a substituted pyrido[3,4-d]pyrimidin-4-one of formula (1) is coupled with CO at a pressure of from about 50 psi to more than 1000 psi in methanol in the presence of a suitable catalyst such as dppf-PdCl₂ or Dppp-Pd(OAc)₂ with a suitable aprotic base such as Et₃N at suitable temperatures of from about room temperature to more than 200° C. to give a methyl ester of formula (2). The methyl ester of formula (2) is then condensed with an amine of formula (3) in the presence of a suitable catalyst such as (CH₃)₃Al in a suitable solvent such as toluene and/or THF at a temperature from about 0° C. to about 100° C. to give an amide of formula (4). The amide of formula (4) is then coupled with a compound of formula (4) to give a compound of Formula I as defined above according to the corresponding procedure described above for Scheme 1.

In Scheme 8, the substituted pyrido[3,4-d]pyrimidin-4-one of formula (1), which is prepared in a manner analogous to that described in Scheme 1 for the preparation of the substituted pyrido[3,4-d]pyrimidin-4-one of formula (6), is selectively deprotonated with a suitable base such as Et₃N, pyridine, sodium carbonate, 1 mole equivalent of sodium methoxide, and the like in a non-nucleophilic solvent such as DMF, THF, and the like and coupled with the compound of formula (2) to give the N-substituted pyrido[3,4-d]pyrimidin-4-one of formula (3). The N-substituted pyrido[3,4-d]pyrimidin-4-one of formula (3) is then coupled with CO at a pressure of from about 50 psi to more than 1000 psi in a suitable aprotic solvent such as THF in the presence of an amine of formula (4) and a suitable catalyst such as dppf-PdCl₂ or Dppp-Pd(OAc)₂ with a suitable aprotic base such as Et₃N at suitable temperatures of from about room temperature to more than 200° C. to give the compound of Formula I as described above.

In Scheme 9, the substituted pyrido[3,4-d]pyrimidin-4-one of formula (1), which is prepared in a manner analogous to that described in Scheme 1 for the preparation of the substituted pyrido[3,4-d]pyrimidin-4-one of formula (6), is coupled with CO at a pressure of from about 50 psi to more than 1000 psi in a suitable aprotic solvent such as THF in the presence of an amine of formula (2) and a suitable catalyst such as dppf-PdCl₂ or Dppp-Pd(OAc)₂ with a suitable aprotic base such as Et₃N at suitable temperatures of from about room temperature to more than 200° C. to give the amide of formula (3). The amide of formula (3) is selectively deprotonated with a suitable base such as Et₃N, pyridine, sodium carbonate, 1 mole equivalent of sodium methoxide, and the like in a non-nucleophilic solvent such as DMF, THF, and the like and coupled with the compound of formula (4) to give the compound of Formula I as described above.

Preparations of particular invention compounds are described below in the compound examples.

### COMPOUND EXAMPLE 1

### 4-[6-(4-methoxy-benzylcarbamoyl)-4-oxo-4H-pyrido[3,4-d]pyrimidin-3-ylmethyl]-benzoic acid

Preparation Method 1:

### Step (a): 6-chloro-pyridin-3-ylamine

A solution of 2-chloro-5-nitropyridine (50.00 g, 315.5 mmol) in THF (400 mL) was treated with Ra Ni (8.0 g), and the reaction mixture was hydrogenated at 56 psi of hydrogen at 100° C. for 20 hours. The reaction mixture was cooled to room temperature and filtered. The filtrate was evaporated, and the resulting solid was triturated with hexanes/ethyl acetate 9:1. The solids were collected by filtration and dried to give 37.61 g of 6-chloro-pyridin-3-ylamine as a brown solid (92.8% yield).

1H NMR (400 MHz, CHLOROFORM-D) d ppm 3.7 (s, 2H), 6.9 (m, 1H), 7.1 (d, J=9.0 Hz, 1H), 7.8 (s, 1H)

MS (APCI) M+1=129.0

### Step (b): (6-chloro-pyridin-3-yl)-carbamic acid tert-butyl ester

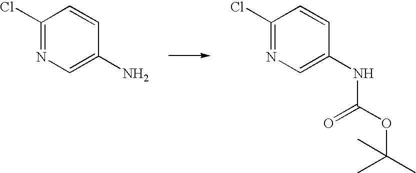

A solution of 6-chloro-pyridin-3-ylamine (37.55 g, 292.1 mmol) in dioxane (150 mL) was treated with di-t-butyldicarbonate (89.0 g, 409 mmol), and the reaction mixture heated at reflux overnight. An additional 3.5 g of di-t-butyldicarbonate was added, and the reaction mixture was heated at reflux for 5 hours. The mixture was cooled to room temperature and evaporated to dryness. The resulting solid was dissolved in methylene chloride, and the solution was passed through a plug of silica gel, eluting with methylene chloride. The eluted solution was evaporated to dryness, and the resulting solid was triturated with hot hexanes, allowed to cool to room temperature, and collected by filtration. The filtercake was washed with hexanes and dried to give 59.25 g of (6-chloro-pyridin-3-yl)-carbamic acid tert-butyl ester as a light pink solid (88.7% yield).

1H NMR (400 MHz, CHLOROFORM-D) d ppm 1.5 (s, 9H), 6.6 (bs, 1H), 7.2 (d, J=8.8 Hz, 1H), 8.0 (d, J=7.3 Hz, 1H), 8.2 (m, 1H)

MS (APCI) M+1=229.1

### Step (c): 5-tert-butoxycarbonylamino-2-chloro-isonicotinic acid

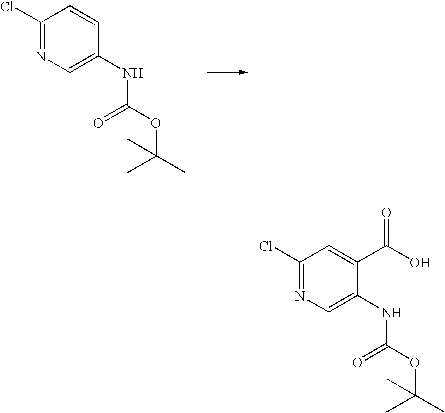

A suspension of (6-chloro-pyridin-3-yl)-carbamic acid tert-butyl ester in 800 mL of ether was treated with TMEDA, and the mixture cooled to −75° C. To this was added 1.6M BuLi in hexanes dropwise while keeping the temperature below −65° C. After the addition was complete, the reaction mixture was allowed to warm to −15° C. to −10° C., and stirred in this temperature range for 2 hours. The reaction mixture was again cooled to −75° C., and dry carbon dioxide was bubbled into the mixture for 3 hours before allowing the reaction to warm to room temperature overnight with continued bubbling of carbon dioxide. The reaction mixture was carefully quenched with 20% aqueous ammonium hydroxide solution (1.8L), the aqueous portion extracted with ether, then acidified to pH 5 using 50% aqueous HCl. The resulting solid was collected by filtration, washed with water, and dried to give 33.07 g (69.3% yield) of 5-tert-butoxycarbonylamino-2-chloro-isonicotinic acid as a light yellow solid.

1H NMR (400 MHz, DMSO-D6) d ppm 1.5 (s, 9H), 7.7 (s, 1H), 9.1 (s, 1H), 10.0 (s, 1H)

MS (APCI) M+1=273.1

### Step (d): 5-amino-2-chloro-isonicotinic acid

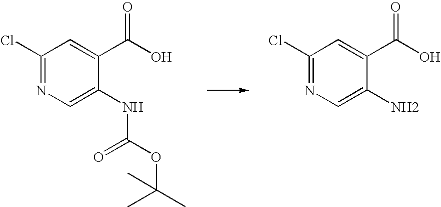

A suspension of 5-tert-butoxycarbonylamino-2-chloro-isonicotinic acid in methylene chloride (600 mL) was treated dropwise at room temperature with TFA until the solid had dissolved into solution (95 mL). The reaction mixture was stirred overnight under nitrogen at room temperature, evaporated to dryness, diluted with water, and the solid collected by filtration. The solid was washed with water, dried under low heat and house vacuum, to afford 19.55 g of 5-amino-2-chloro-isonicotinic acid as a yellow solid (93.6% yield).

1H NMR (400 MHz, DMSO-D6) 8 ppm 7.5 (s, 2H), 8.0 (s, 2H)

MS (APCI) M+1=173.0

### Step (e): 6-chloro-3H-pyrido[3,4-d]pyrimidin-4-one

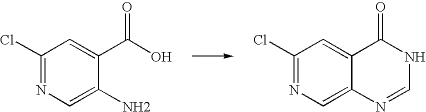

A suspension of 5-amino-2-chloro-isonicotinic acid in formamide (240 mL) was heated at an internal temperature of 140° C. overnight with stirring. The mixture was cooled to room temperature, diluted with water (600 mL), and stirred for 1 hour. The resulting solid was collected by filtration, washed with water, and dried to give 17.20 g of 6-chloro-3H-pyrido[3,4-d]pyrimidin-4-one as a brown solid (83.9% yield).

1H NMR (400 MHz, DMSO-D6) d ppm 7.9 (s, 1H), 8.2 (s, 1H), 8.9 (s, 1H), 12.7 (bs, 1H)

MS (APCI) M+1=182.0

### Step (f): 4-(6-chloro-4-oxo-4H-pyrido[3,4-d]pyrimidin-3-ylmethyl)-benzoic acid tert-butyl ester

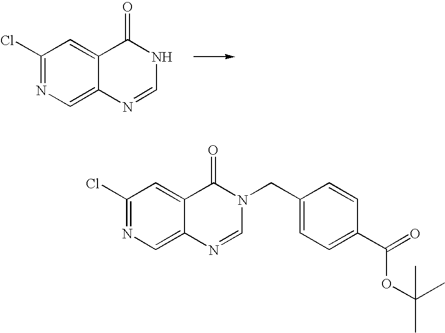

A suspension of 6-chloro-3H-pyrido[3,4-d]pyrimidin-4-one in DMF (230 mL) was treated with cesium carbonate and stirred at room temperature for 1 hour. The mixture was treated with 4-aminomethylbenzoic acid tert-butyl ester (62.2 g, 195 mmol, 0.85 mole equivalents) and reaction mixture solidified almost immediately; an additional 100 mL of DMF was added. The reaction mixture was stirred at room temperature for 2 hours, heated overnight at 60° C., and cooled to room temperature. The mixture was filtered to remove the cesium carbonate, and the filtercake was washed with DMF. Upon standing, a white solid began to form in the filtrate. This solid was collected by filtration, washed with DMF, and then ethyl acetate.

The filtrate was evaporated to dryness, and the resulting solid/oil mixture was treated with ethyl acetate and 1N HCl, giving two layers. The layers were separated, and the organic portion was evaporated to dryness. The residue was triturated with hot hexanes/ethyl acetate 3:1 and cooled to room temperature. The resulting solid was collected by filtration and washed with hexanes/ethyl acetate 3:1. The initial white solid from the cesium carbonate wash was combined with this solid, and the combined material was triturated with hot hexanes/ethyl acetate 3:1, cooled to room temperature, and further cooled in a refrigerator for 45 minutes. The solids were collected by filtration, washed with hexanes/ethyl acetate 4:1, and dried to give 46.32 g of 4-(6-chloro-4-oxo-4H-pyrido[3,4-d]pyrimidin-3-ylmethyl)-benzoic acid tert- butyl ester as a light yellow solid (89.5% yield).

1H NMR (400 MHz, DMSO-D6) δ ppm 1.5 (s, 9H), 3.9 (s, 3H), 5.3 (s, 2H), 7.5 (d, J=8.5 Hz, 2H), 7.8 (d, J=8.5 Hz, 2H), 8.5 (s, 1H), 8.8 (s, 1H), 9.1 (s, 1H)

MS (APCI) M+1=372.1

### Step (g): 3-(4-tert-butoxycarbonyl-benzyl)-4-oxo-3,4 dihydro-pyrido[3,5-d]pyrimidine-6-carboxylic acid methyl ester

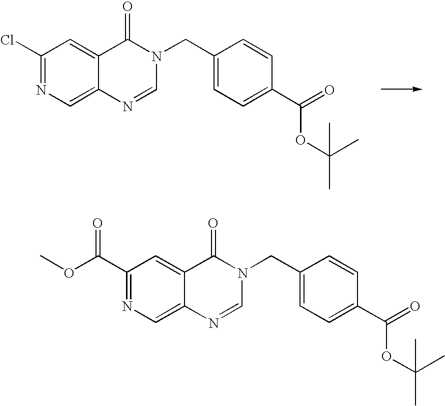

A solution of 4-(6-chloro-4-oxo-4H-pyrido[3,4-d]pyrimidin-3-ylmethyl)-benzoic acid tert- butyl ester in methanol (465mL) was treated with dppf-PdCl2 and triethylamine, then heated at 100° C. at 500 psi of CO for 14.5 hours. The resulting solid was collected by filtration, washed with methanol (100 mL), and washed with hexanes/ethyl acetate 2:1. The resulting filtercake was dried to give 39.29 g of a gray solid.

The filtrate was evaporated onto silica gel, the mesh placed on top of a plug of silica gel, and eluted with ethyl acetate. The filtrate was evaporated, triturated with hexanes/ethyl acetate, the solid collected by filtration and dried to give 5.86 g of a red solid.

The gray and red solids were combined to give 45.15 g of 3-(4-tert-butoxycarbonyl-benzyl)-4-oxo-3,4 dihydro-pyrido[3,5-d]pyrimidine-6-carboxylic acid methyl ester (92.3% yield).

1H NMR (400 MHz, DMSO-D6) δ ppm 1.5 (s, 9H), 3.9 (s, 3H), 5.3 (s, 2H), 7.5 (d, J=8.5 Hz, 2H), 7.8 (d, J=8.5 Hz, 2H), 8.5 (s, 1H), 8.8 (s, 1H), 9.1 (s, 1H)

MS (APCI) M+1=396.1

### Step (h): 4-[6-(4-methoxy-benzylcarbamoyl)-4-oxo-4H-pyrido[3,4-d]pyrimidin-3-ylmethyl]-benzoic acid tert-butyl ester

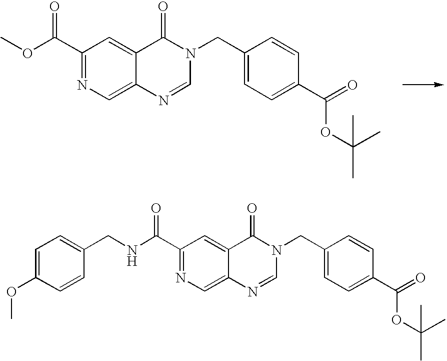

A solution of 4-methoxybenzylamine (17.8 mL) in THF (800 mL) was degassed with nitrogen, and treated with a 2.0M solution of trimethylaluminum in toluene (125 mL) and the solution stirred at room temperature for 7 hours. To this mixture was added 3-(4-tert-butoxycarbonyl-benzyl)-4-oxo-3,4 dihydro-pyrido[3,5-d]pyrimidine-6-carboxylic acid methyl ester in portions over 5 minutes, and the resulting dark solution stirred at room temperature for 4 days. The reaction mixture was carefully quenched with methanol, stirred until gas evolution ceased, then treated with an additional 200 mL of methanol. Stirring then continued for 1 hour. The thick mixture was diluted with THF (300 mL), filtered through a short pad of diatomaceous earth, washed with THF, washed with methanol, and washed with THF again. The filtrate was evaporated to dryness, and the resulting residue was dissolved in hot THF (300 mL) and filtered through a short pad of silica gel by eluting with THF. The filtrate was evaporated, the resulting residue triturated with ether to give a solid. The solid was collected by filtration, washed with ether, and dried to give 50.73 g of 4-[6-(4-methoxy-benzylcarbamoyl)-4-oxo-4H-pyrido[3,4-d]pyrimidin-3-ylmethyl]-benzoic acid tert-butyl ester as an off-white solid (89.0% yield).

1H NMR (400 MHz, DMSO-D6) δ ppm 1.5 (s, 9H), 3.7 (s, 3H), 4.4 (d, J=6.1 Hz, 2H), 5.3 (s, 2H), 6.8 (d, J=8.3 Hz, 2H), 7.3 (d, J=8.5 Hz, 2H), 7.5 (d, J=8.1 Hz, 2H), 7.8 (d, J=8.1 Hz, 2H), 8.5 (s, 1H), 8.8 (s, 1H), 9.1 (s, 1H), 9.3 (t, J=6.1 Hz, 1H)

MS (APCI) M+1=501.2

### Step (i): 4-[6-(4-methoxy-benzylcarbamoyl)-4-oxo-4H-pyrido[3,4-d]pyrimidin-3-ylmethyl]-benzoic acid

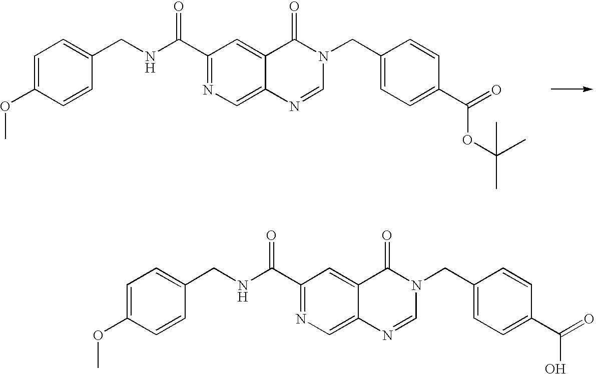

A suspension of 4-[6-(4-methoxy-benzylcarbamoyl)-4-oxo-4H-pyrido[3,4-d]pyrimidin-3-ylmethyl]-benzoic acid tert-butyl ester in 400 mL of methylene chloride was treated dropwise with 125 mL of TFA, then stirred overnight at room temperature. The dark solution was evaporated to dryness, triturated with ether, collected by filtration and dried to give 4-[6-(4-methoxy-benzylcarbamoyl)-4-oxo-4H-pyrido[3,4-d]pyrimidin-3-ylmethyl]-benzoic acid as an amorphous solid with residual TFA and some crystalline product. The solid was chunky. It was crushed with a mortar and pestle, then slurried in 700 mL of acetonitrile at an internal temperature of 50° C. for 1 hour. The suspension was cooled, the solid collected, washed with acetonitrile, and dried to a mud (the filtrate was noted to be deeply colored). The solid was then slurried in 800 mL of methanol, heated to boiling for 5 minutes, then allowed to cool to room temperature. The solid was collected by filtration, washed with methanol, crushed with a mortar and pestle, then dried to give 42.02 g of 4-[6-(4-methoxy-benzylcarbamoyl)-4-oxo-4H-pyrido[3,4-d]pyrimidin-3-ylmethyl]-benzoic acid as an off-white solid (93.6% yield).

1H NMR (400 MHz, DMSO-D6) d ppm 3.7 (s, 3H), 4.4 (d, J=6.3 Hz, 2H), 5.3 (s, 2H), 6.8 (d, J=8.8 Hz, 2H), 7.3 (d, J=8.8Hz, 2H), 7.5 (d, J=8.5 Hz, 2H), 7.9 (d, J=8.5 Hz, 2H), 8.5 (s, 1H), 8.8 (s, 1H), 9.1 (s, 1H), 9.3 (t, J=6.3 Hz, 1H), 12.9 (s, 1H)

MS (APCI) M+1=445.1; mp 245.0-246.0° C.

Preparation Method 2:

It should be appreciated that the starting material and intermediates described above in Preparation Method 1 are also used below in Preparation Method 2. However, reagents, reaction times and temperatures, work-ups, purifications, and the like may differ between Preparation Methods 1 and 2.

### Step (a): Preparation of 6-chloro-pyridin-3-ylamine

A 5 gallon stirred stainless steel pressure reactor was charged with 2-chloro-5-nitropyrimidine (571.8 g, 3.61 moles), 8.5 L of THF, and Raney Nickel (150 g). The vessel was pressurized to 50 psi with hydrogen gas, and the mixture was stirred at room temperature overnight. An aliquot checked by mass spectrometry showed the reaction was complete. The solvent was reduced to about 750 mL and left sitting at room temperature overnight. A first crop of solid had formed in the about 750 mL of solvent remaining. The solid was collected by filtration, and the filtercake was washed 1 time with THF, 2 times with heptane, and dried overnight a vacuum oven at 45° C.

Separately, about 1.5 L of heptane were added to the filtrate from above, and the mixture was refrigerated for 2 hours. A second crop of solids that formed were collected by filtration, and the filtercake was washed 1 time with heptane and dried overnight in the vacuum oven at 45° C.

Meanwhile, the filtrate from the second crop was rotary evaporated, and a residual solid third crop was collected and dried overnight in the vacuum oven at 45° C. The reaction yielded 432 g (93% total yield in 3 crops) of 6-chloro-pyridin-3-ylamine that was sufficiently pure by NMR to carry on in the next reaction without further purification.

δ_{H}(DMSO) 7.64 (1 H, m), 7.03 (1 H, d), 6.93 (1 H, d), 5.44 (2 H, s)

### Step (b): (6-Chloro-pyridin-3-yl)-carbamic acid tert-butyl ester

A 2 L round bottomed flask was charged with 6-chloro-pyridin-3-ylamine (271 g, 2.11 moles), BOC₂O (552 g, 2.53 moles, 1.2 equivalents), and 1 L of dichloroethane. The resulting solution was heated at 65° C. for 10 hours, then allowed to cool to room temperature. The resulting precipitate was collected by filtration, and the filtercake was washed 3 times with dichloroethane. This solid was dried overnight in the vacuum oven at 45° C. Characterization by 1H-NMR indicated this solid was a by-product named N,N′-bis(2-chloro-pyridin-5-yl)-urea. The filtrate was rotary evaporated, and the residual solid was slurried in about 1 L of heptane at 55° C. for 3 hours and then cooled to room temperature. The slurry was filtered, and the filtercake was washed 3 times with heptane and dried over night in the vacuum oven at 45° C. to give 333 g (69% total yield) of a tan solid that was sufficiently pure by NMR to use in the next reaction.

δ_{H} (DMSO) 9.70 (1 H, s), 8.42 (1 H, d), 7.90 (1 H, d), 7.37 (1 H, d), 1.44 (9 H, s)

### Step (c): 5-tert-Butoxycarbonylamino-2-chloro-isonicotinic acid

The reaction was run under Argon. A 20 L jacketed reactor was charged with (6-chloro-pyridin-3-yl)-carbamic acid tert-butyl ester (200 g, 0.875 moles), THF (5 L), and TMEDA (304 mL, 2.3 equivalents, 2 wt % water), and the mixture was cooled to between −70° C. and −75° C. nBuLi (805 mL, 2.5 M in hexanes, 2.3 mole equivalents) was added via dropping funnel at a rate that kept the reaction temperature between −70° C. and −75° C. The resulting brown solution was warmed to −15° C. and stirred for 1 hour. The reaction mixture was then cooled back to −35° C., and a lecture bottle of CO₂ was bubbled through. The reaction mixture was allowed to warm to 20° C. over 2 hours, during which time the solution became an orange slurry. The reaction mixture was stirred at room temperature overnight. The reaction was quenched by the addition of 1.5 L of water. During the quench a precipitate formed in the aqueous layer. The layers were separated, taking the precipitated solid with the aqueous layer. The organic layer was washed once with 1 N NaOH. The aqueous portions were combined, and the pH adjusted to pH 2 with 6 N HCl. The solid was collected by filtration, washed twice with water and dried overnight in the vacuum oven at 45° C. The reaction yielded 143 g (60% total yield) of 5-tert-Butoxycarbonylamino-2-chloro-isonicotinic acid as a tan solid that was sufficiently pure by NMR to use in the next reaction.

δ_{H}(DMSO) 10.06 (1 H, s), 9.07 (1 H, s), 7.71 (1 H, d), 1.42 (9 H, s)

MS [M+H]⁺273

### Step (d): 5-Amino-2-chloro-isonicotinic acid

A 3 L round bottomed flask was charged with 5-tert-butoxycarbonylamino-2-chloro-isonicotinic acid (138 g, 0.51 moles), 1 L of CH₂Cl₂, and 400 mL of TFA. The resulting orange solution was stirred overnight at room temperature. One liter of H₂O was added to the reaction solution, which caused a solid to precipitate out. The solid was collected, washed once with H₂O and dried overnight in the vacuum oven at 45° C. The reaction yielded 69.6 g (80% total yield) of 5-amino-2-chloro-isonicotinic acid as a pale yellow solid, which was pure enough by NMR to use in the next reaction.

δ_{H} (DMSO) 7.99 (1 H, d), 7.45 (1 H, d)

MS [M+H]⁺173

### Step (e) 6-Chloro-3H-pyrido[3,4-d]pyrimidin-4-one

A 1 L round bottomed flask was charged with 5-amino-2-chloro-isonicotinic acid (69.5 g, 0.40 moles), formamidi [symbol] e acetate (84g, 0.81 moles, 2 mole equivalents), and 600 mL of methoxyethanol. The resulting solution was heated at reflux for 18 hours. After cooling to 5° C., a precipitate was collected by filtration, washed twice with methoxyethanol, and dried overnight in the vacuum oven at 45° C. The reaction yielded 67 g (92% total yield) of 6-chloro-3H-pyrido[3,4-d]pyrimidin-4-one as a tan solid that was sufficiently pure by NMR to use in the next reaction.

δ_{H} (DMSO) 12.70 (1 H, s), 8.86 (1 H, d), 8.19 (1 H, s), 7.93 (1 H, d)

MS [M+H]⁺182

### Step (f): 4-(6-Chloro-4-oxo-4H-pyrido[3,4-d]pyrimidin-3-ylmethyl)-benzoic acid tert-butyl ester

A 2 L round bottomed flask was charged with 6-chloro-3H-pyrido[3,4-d]pyrimidin-4-one (61.9 g, 0.34 moles), Cs₂CO₃ (155 g, 0.48 moles, 1.4 mole equivalents), and 900 mL of DMF. The slurry was stirred for 5 minutes, then t-butyl-4-bromomethylbenzoate (129 g, 0.48 moles, 1.4 mole equivalents) was added, and stirring of the resulting thick slurry was continued. After 15 minutes HPLC (C18, 4: 1/CH₃CN: 0.1% TFA, 254 nm, 1 mL/min) showed less than 3% of 6-chloro-3H-pyrido[3,4-d]pyrimidin-4-one remained. After 30 minutes the reaction was complete. Added 450 mL of H₂O to the slurry, and collected the resulting solid by filtration. The solid was washed twice with 2:1/DMF: H₂O, once with H₂O, and dried overnight in the vacuum oven at 45° C. The reaction yielded 124 g (98% total) of 4-(6-chloro-4-oxo-4H-pyrido[3,4-d]pyrimidin-3-ylmethyl)-benzoic acid tert-butyl ester as a white solid that was 99% pure by HPLC.

δ_{H} (DMSO) 8.94 (1 H, d), 8.71 (1 H, s), 7.99 (1 H, d), 7.83 (2 H, d), 7.45 (2 H, d), 5.26 (2 H, s), 1.49 (9 H, s)

MS [M+H]⁺372

HPLC 99.02%, RT 2.90 min; YMC Pack Pro C18 4.6×150 mm, 3 μ; A: 0.05% TFA in H₂O, B: 0.05% TFA in CH₃CN; 10% B to 95% B over 15 minutes, hold for 5 minutes; λ 240 nm, 1 ml/min

### Step (g): 4-(6-Methoxycarbonyl-4-oxo-4H-pyrido[3,4-d]pyrimidin-3-ylmethyl)-benzoic acid tert-butyl ester

A 2 L High Pressure vessel was charged with 4-(6-chloro-4-oxo-4H-pyrido[3,4-d]pyrimidin-3-ylmethyl)-benzoic acid tert-butyl ester (132 g, 0.35 moles), DPPF-PDCL2 (2.89 g, 1 mol %), Et₃N (98 mL, 2 mole equivalents), and 1.1 L of methanol. The vessel was sealed, purged and then pressurized to 500 psi with CO. The reaction mixture was stirred and heated at 100° C. for 14 hours. After cooling to room temperature, mass spectrometry showed the reaction was complete. The resulting precipitate was collected and washed with methanol until the wash came through the filtercake clear. The first crop of solid was dried overnight in the vacuum oven at 45° C. The filtrate and washes were reduced in volume until a thick slurry formed. The solid was collected by filtration, and washed with methanol until the wash came through the filtercake clear. The second crop of solid was dried overnight in the vacuum oven at 45° C. The reaction yielded 124 g (89% total yield) of 4-(6-methoxycarbonyl-4-oxo-4H-pyrido[3,4-d]pyrimidin-3-ylmethyl)-benzoic acid tert-butyl ester as a purple solid (in two crops) that was sufficiently pure by NMR to use in the next step.

δ_{H} (DMSO) 9.11 (1 H, s), 8.80 (1 H, s), 8.49 (1 H, s), 7.80 (2 H, d), 7.44 (2 H, d), 5.26 (2 H, s), 3.87 (3 H, s), 1.46 (9 H, s)

MS [M+H]⁺39

### Step (h): 4-[6-(4-Methoxy-benzylcarbamoyl)-4-oxo-4H-pyrido[3,4-d] pyrimidino-3-ylmethyl]benzoic acid tert-butyl ester

The reaction was run under an argon atmosphere. A 3 L round bottomed flask was charged with 4-methoxybenzylamine (49.4 mL, 0.38 ml, 1.2 mole equivalents) and 250 mL of THF. (CH₃)₃Al (346 mL, 2.2 mole equivalents, 2.0 M in toluene) was added via dropping funnel at a rate to keep the temperature at or below 40° C. The addition took about 45 minutes, after which the resulting solution was stirred for 30 minutes. The 4-(6-methoxycarbonyl-4-oxo-4H-pyrido[3,4-d]pyrimidin-3-ylmethyl)-benzoic acid tert-butyl ester (124 g, 0.31 moles) was dissolved in 1.4 L of THF. An insoluble black solid (assumed to be palladium from the previous reaction) was filtered off. The filtrate solution was added to the reaction mixture via dropping funnel at a rapid rate. Degassing began as soon as the addition began, the brown reaction solution turned black, and the temperature rose to 35° C. After the degassing had ceased, analysis of the reaction mixture by mass spectrometry showed some 4-(6-methoxycarbonyl-4-oxo-4H-pyrido[3,4-d]pyrimidin-3-ylmethyl)-benzoic acid tert-butyl ester remained. After stirring an additional 30 minutes, mass spectrometry showed the reaction was complete. The reaction vessel was placed in an ice bath, and the reaction mixture was quenched using 470 mL of 0.67 M HCl. A precipitate (presumed to be alumina salts) formed in the aqueous layer. The layers were separated and the organic layer was washed twice with 0.67 M HCl, and once with H₂O. The combined aqueous layers were washed twice with EtOAc. The organic portions were combined, dried over MgSO₄, filtered and rotary evaporated. The resulting solid was dried overnight in the vacuum oven at 45° C. The reaction yielded 154 g (98% total yield) of 4-[6-(4-methoxy-benzylcarbamoyl)-4-oxo-4H-pyrido[3,4-d]pyrimidino-3-ylmethyl]benzoic acid tert-butyl ester as an off white solid that was 98.5% pure by HPLC (C18, 90:10/A: B to 15:85/A: B over 15 minutes then hold 2 minutes, A=9:1/H2O: CH3CN with 0.2% perchloric acid, B=CH₃CN, 240 nm, 1 mL/minute). Microanalysis showed palladium present at 16 ppm, aluminum present at 3 ppm.

δ_{H} (DMSO) 9.32 (1 H, t), 9.05 (1 H, d), 8.75 (1 H, s), 8.50 (1 H, d), 7.81 (2 H, d), 7.44 (2 H, d), 7.22 (2 H, d), 6.81 (2H, d), 5.25 (2 H, s), 4.40 (2 H, d), 3.66 (3 H), s), 1.46 (9 H, s)

MS [M+H]⁺501

Microanalysis Theoretical: C, 67.19; H, 5.64; N, 11.19; Found: C, 67.07; H, 5.65; N, 11.06; Pd, 16 ppm; Al, 3 ppm.

HPLC 98.33 %, RT 14.96 min; YMC Pack Pro C18 4.6×150 mm, 3 μ; A: 0.05%

TFA in H₂O, B: 0.05% TFA in CH₃CN; 10% B to 95% B over 15 minutes, hold for 5 minutes; λ 240 nm, 1 ml/min

### Step (i): 4-[6-(4-Methoxy-benzylcarbamoyl)-4-oxo-4H-pyrido[3,4-d]pyrimidino-3-ylmethyl]benzoic acid

A 3L round bottomed flask was charged with 4-[6-(4-methoxy-benzylcarbamoyl)-4-oxo-4H-pyrido[3,4-d]pyrimidino-3-ylmethyl]benzoic acid tert-butyl ester (150 g, 0.30 moles) and 1.5 L of CH₃CN. To the resulting slurry was added TFA (232 ml, 10 mole equivalents). The orange solution was heated to 50° C. After about 15 minutes a precipitate started to form. After 5 hours HPLC (C18, 90:10/A: B to 15:85/A: B over 15 minutes then hold 2 minutes, A=9:1/H₂O: CH3CN with 0.2% perchloric acid, B=CH₃CN, 240 nm, 1 mL/minute) showed the reaction was complete. The slurry was cooled to 5° C., and the solid was collected by filtration, washed twice with CH₃CN, and dried overnight in the vacuum oven at 45° C. The reaction yielded 123 g (92% total yield) of 4-[6-(4-methoxy-benzylcarbamoyl)-4-oxo-4H-pyrido[3,4-d]pyrimidino-3-ylmethyl]benzoic acid as a white solid. Microanalysis showed palladium present at 9 ppm, aluminum at 3 ppm. Powder X-ray Diffraction showed the solid was Crystal Form 1 of 4-[6-(4-methoxy-benzylcarbamoyl)-4-oxo-4H-pyrido[3,4-d]pyrimidino-3-ylmethyl]benzoic acid.

δ_{H} (DMSO) 12.91 (1 H, s), 9.32 (1 H, t), 9.06 (1 H, d), 8.76 (1 H, s), 8.51 (1 H), d), 7.86 (2 H, m), 7.44 (2 H, d), 7.22 (2 H, m), 6.81 (2 H, m), 5.26 (2 H, s), 4.40 (2 H, d), 3.66 (3 H, s)

MS [M+H]⁺445

Microanalysis Theoretical: C, 64.86; H, 4.54; N, 12.61; Found: C, 64.62; H, 4.47; N, 12.62

HPLC 98.83%, RT 10.4 min; YMC Pack Pro C18 4.6×150 mm, 3 μ; A: 0.05%

TFA in H₂O, B: 0.05% TFA in CH₃CN; 10% B to 95% B over 15 minutes, hold for 5 minutes; λ 240 nm, 1 ml/min

The final form of 4-[6-(4-methoxy-benzylcarbamoyl)-4-oxo-4H-pyrido[3,4-d]pyrimidin-3-ylmethyl]-benzoic acid prepared according to the methods of Compound Example 1 Preparation Methods 1 and 2 is a single crystalline form. However, other crystalline forms of 4-[6-(4-methoxy-benzylcarbamoyl)-4-oxo-4H-pyrido[3,4-d]pyrimidin-3-ylmethyl]-benzoic acid are expected.

Powder x-ray diffraction patterns for Crystal Form 1 of 4-[6-(4-methoxy-benzylcarbamoyl)-4-oxo-4H-pyrido[3,4-d]pyrimidin-3-ylmethyl]-benzoic acid were collected using a Rigaku powder X-ray diffractometer utilizing a copper target or a Bruker D8 powder X-ray diffractometer, also utilizing a copper target. Typical scanning parameters for the Rigaku powder X-ray diffractometer were 3°-50° 2-theta at a scanning rate of 1° per minute. Typical scanning parameters for the Bruker D8 powder X-ray diffractometer were 6°-41° 2-theta collected in 60 seconds. The Bruker system is a higher throughput system, but provides lower resolution and a smaller 2-Theta scanning range than the Rigaku system.

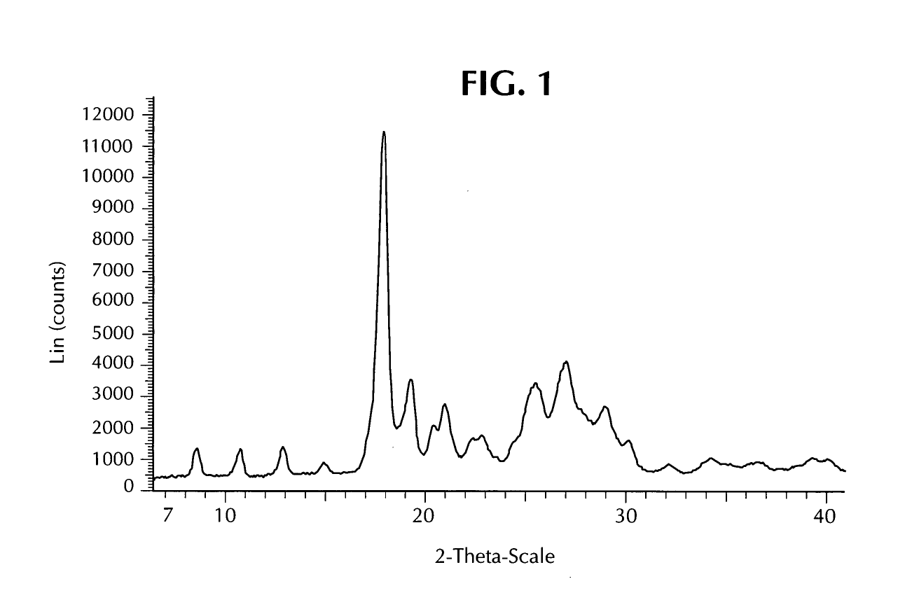

The x-ray powder diffraction pattern (“pXRD”) for Crystal Form 1 of 4-[6-(4-methoxy-benzylcarbamoyl)-4-oxo-4H-pyrido[3,4-d]pyrimidin-3-ylmethyl]-benzoic acid that was collected on the Bruker D8 powder X-ray diffractometer is shown graphically in FIG. 1 as a plot of 2-Theta values, expressed in degrees, on the x-axis versus Linear intensity, expressed as counts, on the y-axis. The pXRD of FIG. 1 was then characterized by the values shown below in pXRD Table 1 in the columns labelled “Peak No.,” “2-Theta,”, “d(A),” “Peak Intensity,” “P %,” “Area,” “Area %,”, and “FWHM”:

PXRD TABLE 1

| Peak | 2-Theta | | Peak | | | | |
|---|---|---|---|---|---|---|---|
| No. | (deg) | d(Å) | Intensity | P % | Area | Area % | FWHM |
| 1 | 8.613 | 10.258 | 1560 | 13.4 | 13.68 | 9.2 | 0.391 |
| 2 | 10.782 | 8.198 | 1504 | 12.9 | 15.18 | 10.2 | 0.405 |
| 3 | 12.953 | 6.829 | 1504 | 12.9 | 16.45 | 11.1 | 0.399 |
| 4 | 15.008 | 5.898 | 1034 | 8.9 | 11.85 | 8.0 | 0.448 |
| 5 | 17.892 | 4.953 | 11637 | 100.0 | 148.4 | 100.0 | 0.534 |
| 6 | 19.287 | 4.598 | 3779 | 32.5 | 51.99 | 35.0 | 0.497 |
| 7 | 20.468 | 4.336 | 2369 | 20.4 | 20.61 | 13.9 | 0.294 |
| 8 | 21.023 | 4.222 | 2951 | 25.4 | 36.2 | 24.4 | 0.418 |
| 9 | 22.85 | 3.888 | 1993 | 17.1 | 48.45 | 32.6 | 0.989 |
| 10 | 25.542 | 3.485 | 3591 | 30.9 | 75.6 | 50.9 | 0.86 |
| 11 | 26.996 | 3.3 | 4361 | 37.5 | 112.6 | 75.9 | 0.767 |
| 12 | 29.05 | 3.071 | 2933 | 25.2 | 53.03 | 35.7 | 0.573 |
| 13 | 30.105 | 2.966 | 1805 | 15.5 | 23.73 | 16.0 | 0.431 |

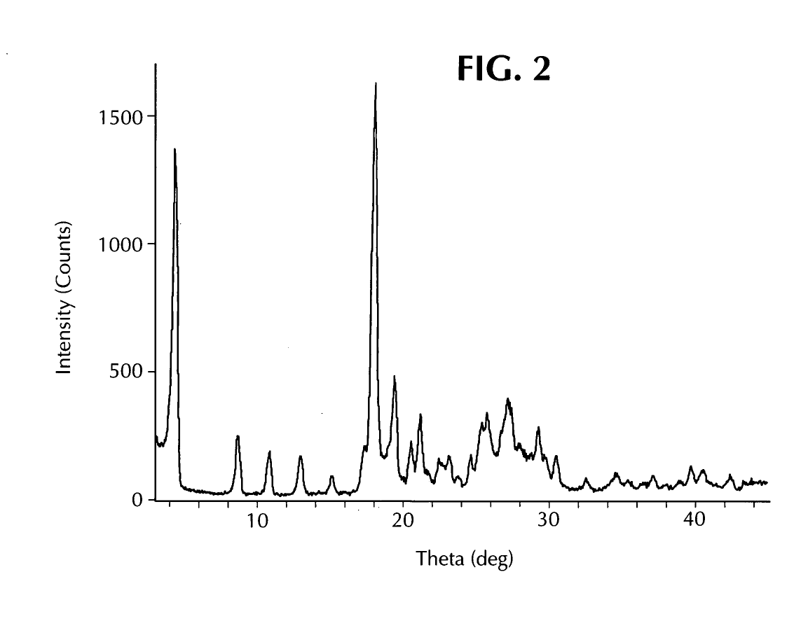

The pXRD of Crystal Form 1 of 4-[6-(4-methoxy-benzylcarbamoyl)-4-oxo-4H-pyrido[3,4-dlpyrimidin-3-ylmethyl]-benzoic acid that was collected on the Rigaku powder X-ray diffractometer is shown graphically in FIG. 2 as a plot of 2-Theta values, expressed in degrees, on the x-axis versus peak intensity, expressed in counts, on the y-axis. The pXRD of FIG. 2 was then characterized by the values shown below in pXRD Table 2 in the columns labelled “Peak No.,” “2-Theta,”, “d(A),” “Peak Intensity,” “P %,” “Area,” “Area %,”, and “FWHM”:

PXRD TABLE 2

| Peak | 2-Theta | | Peak | | | | |
|---|---|---|---|---|---|---|---|
| No. | (deg) | d(Å) | Intensity | P % | Area | Area % | FWHM |
| 1 | 4.363 | 20.2347 | 1337 | 84.9 | 641 | 77.6 | 0.383 |
| 2 | 8.649 | 10.215 | 227 | 14.4 | 89 | 10.7 | 0.31 |
| 3 | 10.84 | 8.1548 | 168 | 10.7 | 59 | 7.1 | 0.28 |
| 4 | 12.927 | 6.8429 | 149 | 9.5 | 63 | 7.6 | 0.338 |
| 5 | 15.09 | 5.8663 | 69 | 4.4 | 26 | 3.1 | 0.3 |
| 6 | 17.36 | 5.1041 | 186 | 11.8 | 96 | 11.6 | 0.411 |
| 7 | 18.001 | 4.9237 | 1575 | 100 | 826 | 100 | 0.419 |
| 8 | 19.439 | 4.5625 | 402 | 25.5 | 165 | 19.9 | 0.327 |
| 9 | 20.595 | 4.3091 | 116 | 7.4 | 28 | 3.3 | 0.187 |
| 10 | 21.202 | 4.187 | 216 | 13.7 | 64 | 7.7 | 0.235 |
| 11 | 22.486 | 3.9508 | 88 | 5.6 | 31 | 3.7 | 0.275 |
| 12 | 23.162 | 3.8369 | 107 | 6.8 | 51 | 6.1 | 0.375 |
| 13 | 24.679 | 3.6044 | 115 | 7.3 | 41 | 4.9 | 0.281 |
| 14 | 25.4 | 3.5037 | 228 | 14.5 | 147 | 17.8 | 0.514 |
| 15 | 25.76 | 3.4556 | 183 | 11.6 | 92 | 11 | 0.399 |
| 16 | 26.801 | 3.3236 | 90 | 5.7 | 31 | 3.8 | 0.276 |
| 17 | 27.16 | 3.2805 | 214 | 13.6 | 141 | 17 | 0.526 |
| 18 | 29.278 | 3.0478 | 135 | 8.6 | 43 | 5.1 | 0.251 |
| 19 | 30.444 | 2.9337 | 128 | 8.1 | 69 | 8.3 | 0.429 |

In pXRD Tables 1 and 2, “Peak No.” means the consecutive number of the peak for which a 2-Theta value is reported, “2-Theta (deg)” means the scanning parameter 2-Theta, expressed in degrees, “d(Å)” means the d-spacing in the crystal lattice, expressed in angstroms, “Peak Intensity” means the peak intensity expressed in counts, “P %” means the peak intensity relative to the most intense peak, expressed as a percentage, “Area” means the integrated area under the peak, “Area %” means the integrated area under the peak relative to the integrated area under the most intense peak, expressed as a percentage, and “FWHM” means full-width/half maximum or the width in degrees of the peak at half of the peak's maximum intensity.

### COMPOUND EXAMPLES 1.1-1.5

Compound Examples 1.1-1.5 are cation salts of the compound of Compound Example 1 that have been prepared according to the general procedure described below.

One mole equivalent of monovalent cation (e.g., Na⁺, K⁺, choline (i.e., [HOCH₂CH₂N(CH₃)₃]⁺) or one half mole equivalent of divalent cation (e.g., Ca⁺² or Mg⁺²) dissolved in water or other suitable solvent such as aqueous DMSO, aqueous DMF, methanol, and the like, was added to a solution of 4-[6-(4-methoxy-benzylcarbamoyl)-4-oxo-4H-pyrido [3,4-d]pyrimidin-3-ylmethyl]-benzoic acid in THF:water (60:40) with vigorous stirring. Stirring was continued for 12-16 hours at 40° C. Any precipitates were collected by filtration and allowed to dry in a vacuum desiccator or in a vacuum oven at 40° C. If after 16 hours the solution remained clear, the salts were isolated by addition of a co-solvent to cause precipitation or by evaporation of the solvent. The salts obtained were analyzed by pXRD, TGA and DSC.

Salts that were prepared according to this procedure are listed below in Compound Table 1 in the column “Salt form.”

Compound Table 1.

| Example | | Example | Salt | Exam- | Salt |
|---|---|---|---|---|---|
| No. | Salt form | No. | form | ple No. | form |
| 1.1 | ½ Ca⁺² | 1.2 | 0.50 | 1.3 | Na⁺ |
| | | | Mg⁺² | | |
| 1.4 | [HOCH₂CH₂N(CH₃)₃]⁺ | 1.5 | K⁺ | | |

### COMPOUND EXAMPLE 1.1

### 4- [6-(4-methoxy-benzylcarbamoyl)-4-oxo-4H-pyrido[3,4-d]pyrimidin-3-ylmethyl]-benzoic acid crystalline hemi calcium salt:

pXRD (Bruker D8 instrument) angle 2-Theta (degrees), d-value (angstrom):

| Angle 2-Theta | d-value |
|---|---|
| (degrees) | (Angstroms) |
| 8.03 | 11.00141 |
| 9.683 | 9.12635 |
| 10.178 | 8.684 |
| 13.212 | 6.69591 |
| 13.805 | 6.40952 |
| 14.75 | 6.00069 |
| 16.044 | 5.51951 |
| 17.649 | 5.02119 |
| 19.463 | 4.55708 |
| 20.754 | 4.27643 |
| 21.575 | 4.11544 |
| 22.817 | 3.89417 |
| 23.485 | 3.78489 |
| 24.146 | 3.68273 |
| 27.926 | 3.19231 |
| 30.302 | 2.94718 |
| 30.886 | 2.89274 |
| 32.581 | 2.746 |
| 35.057 | 2.55753 |
| 36.088 | 2.48681 |
| 37.489 | 2.39703 |

### COMPOUND EXAMPLE 1.2

### 4-[6-(4-methoxy-benzylcarbamoyl)-4-oxo-4H-pyrido[3,4-d]pyrimidin-3-ylmethyl]-benzoic acid crystalline hemi magnesium salt:

pXRD (Bruker D8 instrument) angle 2-Theta (degrees), d-value (angstrom):

| Angle 2-Theta | d-value |
|---|---|
| (degrees) | (Angstroms) |
| 8.202 | 10.77152 |
| 8.707 | 10.1469 |
| 9.376 | 9.42439 |
| 12.437 | 7.11089 |
| 12.998 | 6.80562 |
| 14.571 | 6.07391 |
| 15.376 | 5.75774 |
| 16.311 | 5.42978 |
| 17.43 | 5.08381 |
| 18.437 | 4.80829 |
| 20.131 | 4.40735 |
| 21.38 | 4.15249 |
| 23.649 | 3.75901 |
| 25.33 | 3.5133 |
| 25.991 | 3.42539 |
| 28.006 | 3.18334 |

### COMPOUND EXAMPLE 1.3

### 4-[6-(4-methoxy-benzylcarbamoyl)-4-oxo-4H-pyrido[3,4-d]pyrimidin-3-ylmethyl]-benzoic acid amorphous sodium salt:

### COMPOUND EXAMPLE 1.4

### 4-[6-(4-methoxy-benzylcarbamoyl)-4-oxo-4H-pyrido[3,4-d]pyrimidin-3-ylmethyl]-benzoic acid amorphous cholinie sat, deliquescent:

### COMPOUND EXAMPLE 1.5

### 4- [6-(4-methoxy-benzylcarbamoyl)-4-oxo-4H-pyrido[3,4-d]pyrimidin-3-ylmethyl]-benzoic acid amorphous potassium salt:

### COMPOUND EXAMPLE 2

### 4-[6-(3-methoxy-benzylcarbamoyl)-4-oxo-4H-pyrido[3,4-d]pyrimidin-3-ylmethyl]-benzoic acid

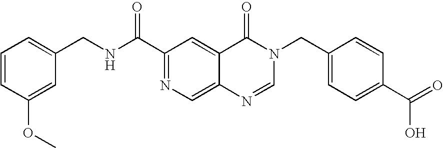

This compound was synthesized in a manner analogous to the procedure described in Compound Example 1 by replacing 4-methoxybenzylamine with 3-methoxybenzylamine.

1H NMR (400 MHz, DMSO-D6) delta (ppm) 3.7 (s, 3H), 4.5 (d, J=6.3 Hz, 2H), 5.3 (s, 2H), 6.8 (d, J=12.0 Hz, 1H), 6.9 (m, 2H), 7.2 (t, J=8.1 Hz, 1H), 7.5 (d, J=8.5 Hz, 2H), 7.9 (d, J=8.5 Hz, 2H), 8.5 (s, 1H), 8.8 (s, 1H), 9.1 (s, 1H), 9.4 (t, J=6.5 Hz, 1H), 12.9 (s, 1H)

mp 202.0-203.0° C.

### COMPOUND EXAMPLE 3

### 4-{4-oxo-6-[(pyridin-3-ylmethyl)-carbamoyl]-4H-pyrido[3,4-d]pyrimidin-3-ylmethyl}-benzoic acid

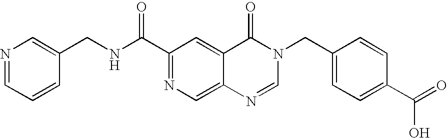

This compound was synthesized in a manner analogous to the procedure described in Compound Example 1 by replacing 4-methoxybenzylamine with pyridin-3-ylmethylamine.

1H NMR (400 MHz, DMSO-D6) delta (ppm) 4.6 (d, J=6.3 Hz, 2H), 5.3 (s, 2H), 7.5 (d, J=8.5 Hz, 2H), 7.7 (dd, J=7.7, 5.5 Hz, 1H), 7.9 (d, J=8.3 Hz, 2H), 8.2 (d, J=8.1 Hz, 1H), 8.5 (s, 1H), 8.6 (d, J=4.9 Hz, 1H), 8.7 (s, 1H), 8.8 (s, 1H), 9.1 (s, 1H), 9.7 (t, J=6.3 Hz, 1H)

mp 156.0-157.0° C.

### COMPOUND EXAMPLE 4

### 4- [6-(4-chloro-benzylcarbamoyl)-4-oxo-4H-pyrido [3,4-d]pyrimidin-3-ylmethyl]-benzoic acid

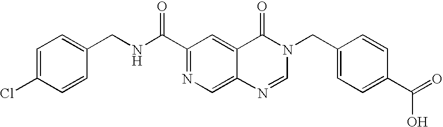

This compound was synthesized in a manner analogous to the procedure described in Compound Example 1 by replacing 4-methoxybenzylamine with 4-chlorobenzylamine.

1H NMR (400 MHz, DMSO-D6) delta (ppm) 4.5 (d, J=6.3 Hz, 2H), 5.3 (s, 2H), 7.3 (s, 4H), 7.5 (d, J=8.5 Hz, 2H), 7.9 (m, J=8.5Hz, 2H), 8.5 (s, 1H), 8.8 (s, 1H), 9.1 (s, 1H), 9.5 (t, J=6.5 Hz, 1H), 12.9 (bs, 1H)

mp 254.0-255.0° C.

### COMPOUND EXAMPLE 5

### 4-{4-oxo-6-[(pyridin-4-ylmethyl)-carbamoyl]-4H-pyrido[3,4-d]pyrimidin-3-ylmethyl}-benzoic acid

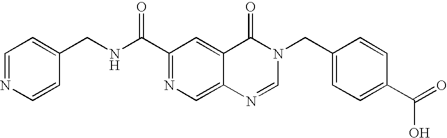

This compound was synthesized in a manner analogous to the procedure described in Compound Example 1 by replacing 4-methoxybenzylamine with pyridin-4-ylmethylamine.

1H NMR (400 MHz, DMSO-D6) delta (ppm) 4.6 (d, J=6.1 Hz, 2H), 5.3 (s, 2H), 7.5 (d, J=8.1 Hz, 2H), 7.6 (d, J=5.4 Hz, 2H), 7.9 (d, J=8.1 Hz, 2H), 8.5 (s, 1H), 8.6 (s, 2H), 8.8 (s, 1H), 9.1 (s, 1H), 9.7 (t, J=6.2 Hz, 1H), 13.0 (bs, 1H) mp>230° C.

MS (APCI) M+1=416.1

### COMPOUND EXAMPLE 6

### 4-{6-[(2-methoxy-pyridin-4-ylmethyl)-carbamoyl]-4-oxo-4H-pyrido [3,4-d]pyrimidin-3-ylmethyl}-benzoic acid

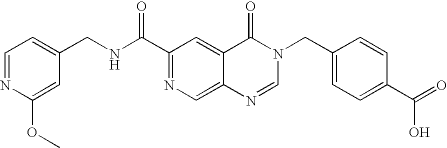

This compound was synthesized in a manner analogous to the procedure described in Compound Example 1 by replacing 4-methoxybenzylamine with (2-methoxypyridin-4-yl)methylamine.

1H NMR (400 MHz, DMSO-D6) delta (ppm) 3.8 (s, 3H), 4.5 (d, J=6.3 Hz, 2H), 5.3 (s, 2H), 6.7 (s, 1H), 6.9 (d, J=5.1 Hz, 1H), 7.5 (d, J=8.3 Hz, 2H), 7.9 (d, J=8.3 Hz, 2H), 8.1 (d, J=5.4 Hz, 1H), 8.5 (s, 1H), 8.8 (s, 1H), 9.1 (s, 1H), 9.6 (t, J=6.2 Hz, 1H), 12.9 (bs, 1H)

mp>230° C.

MS (APCI) M+1=446.1

### COMPOUND EXAMPLE 7

### 4-[6-(4-methylsulfanyl-benzylcarbamoyl)-4-oxo-4H-pyrido[3,4-d]pyrimidin-3-ylmethyl]-benzoic acid

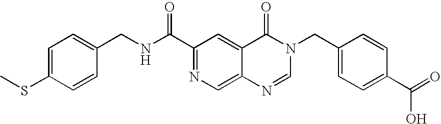

This compound was synthesized in a manner analogous to the procedure described in Compound Example 1 by replacing 4-methylsulfanylbenzylamine with benzylamine.

1H NMR (400 MHz, DMSO-D6) delta (ppm) 2.4 (s, 3H), 4.4 (d, J=6.3 Hz, 2H), 5.3 (s, 2H), 7.2 (d, J=8.1 Hz, 2H), 7.3 (m, 2H), 7.5 (d, J=8.3 Hz, 2H), 7.9 (d, J=8.1 Hz, 2H), 8.5 (s, 1H), 8.8 (s, 1H), 9.1 (s, 1H), 9.5 (m, J=6.2, 6.2 Hz, 1H), 13.0 (bs, 1H)

mp>230° C.

MS (APCI) M+1=461.1

### COMPOUND EXAMPLE 8

### 4-[6-(4-fluoro-benzylcarbamoyl)-4-oxo-4H-pyrido[3,4-d]pyrimidin-3-ylmethyl]-benzoic acid

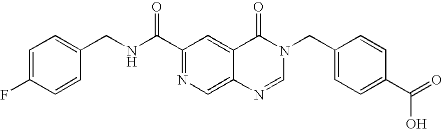

This compound was synthesized in a manner analogous to the procedure described in Compound Example 1 by replacing 4-methoxybenzylamine with 4-fluorobenzylainine.

1H NMR (400 MHz, DMSO-D6) delta (ppm) 4.5 (d, J=6.3 Hz, 2H), 5.3 (s, 2H), 7.1 (t, J=8.8 Hz, 2H), 7.4 (m, 2H), 7.5 (d, J=8.1 Hz, 2H), 7.9 (d, J=8.1 Hz, 2H), 8.5 (s, 1H), 8.8 (s, 1H), 9.1 (s, 1H), 9.5 (t, J=6.3 Hz, 1H), 13.0 (bs, 1H)

mp>230° C.

MS (APCI) M+1=433.1

### COMPOUND EXAMPLE 9

### 4-[6-benzylcarbamoyl)-4-oxo-4H-pyrido[3,4-d]pyrimidin-3-ylmethyl]-benzoic acid

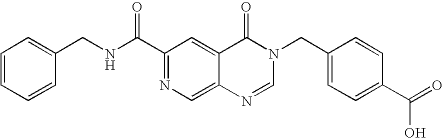

This compound was synthesized in a manner analogous to the procedure described in Compound Example 1 by replacing 4-methoxybenzylamine with benzylamine.

1H NMR (400 MHz, DMSO-D6) delta (ppm) 4.5 (d, J=6.3 Hz, 2H), 5.3 (s, 2H), 7.3 (m, 5H), 7.5 (d, J=8.3 Hz, 2H), 7.9 (d, J=8.3 Hz, 2H), 8.5 (s, 1H), 8.8 (s, 1H), 9.1 (s, 1H), 9.4 (t, J=6.3 Hz, 1H), 12.9 (bs, 1H)

mp=241.0-242.0° C.

MS (APCI) M+1=415.1

### COMPOUND EXAMPLE 10

### 4-[6-(3-chloro-benzylcarbamoyl)-4-oxo-4H-pyrido[3,4-d]pyrimidin-3-ylmethyl]-benzoic acid

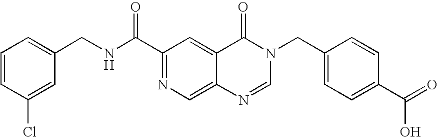

This compound was synthesized as previously described in Compound Example 1, Step (g) using 3-(4-tert-butoxycarbonyl-benzyl)-4-oxo-3,4-dihydro-pyrido[3,5-d]pyrimidine-6-carboxylic acid methyl ester and 3-chlorobenzylamine in place of 4-methoxybenzylamine.

1H NMR (400 MHz, DMSO-D6) delta (ppm) 4.5 (d, J=6.6 Hz, 2H), 5.3 (s, 2H), 7.3 (m, 4H), 7.5 (d, J=8.5 Hz, 2H), 7.9 (d, J=8.5 Hz, 2H), 8.5 (s, 1H), 8.8 (s, 1H), 9.1 (s, 1H), 9.5 (t, J=6.3 Hz, 1H), 12.9 (s, 1H)

mp=227.0-228.0° C.

MS(APCI) M+1=449.1

### COMPOUND EXAMPLE 11

### 4-[6-(3-fluoro-benzylcarbamoyl)-4-oxo-4H-pyrido[3,4-d]pyrimidin-3-ylmethyl]-benzoic acid

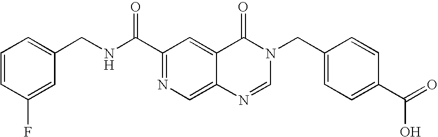

This compound was synthesized as previously described in Compound Example 1, Step (g) using 3-(4-tert-butoxycarbonyl-benzyl)-4-oxo-3,4-dihydro-pyrido[3,5-d]pyrimidine-6-carboxylic acid methyl ester and 3-fluorobenzylamine in place of 4-methoxybenzylamine.

1H NMR (400 MHz, DMSO-D6) delta (ppm) 4.5 (d, J=6.3 Hz, 2H), 5.3 (s, 2H), 7.0 (m, 1H), 7.1 (m, 2H), 7.3 (m, 1H), 7.5 (d, J=8.3 Hz, 2H), 7.9 (d, J=8.5 Hz, 2H), 8.5 (s, 1H), 8.8 (s, 1H), 9.1 (s, 1H), 9.5 (t, J=6.3 Hz, 1H), 12.9 (bs, 1H)

MS(APCI) M+1=433.1.

mp=243.0-244.0° C.

### COMPOUND EXAMPLE 12

### 4-[4-oxo-6-(4-trifluoromethyl-benzylcarbamoyl)-4H-pyrido[3,4-d]pyrimidin-3-ylmethyl]-benzoic acid

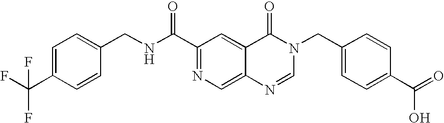

This compound was synthesized as previously described in Compound Example 1, Step (g) using 3-(4-tert-butoxycarbonyl-benzyl)-4-oxo-3,4-dihydro-pyrido[3,5-d]pyrimidine-6-carboxylic acid methyl ester and 4-trifluoromethylbenzylamine in place of 4-methoxybenzylamine.

1H NMR (400 MHz, DMSO-D6) delta (ppm) 4.6 (d, J=6.3 Hz, 2H), 5.3 (s, 2H), 7.5 (d, J=8.5 Hz, 2H), 7.5 (d, 2H), 7.7 (d, J=8.1 Hz, 2H), 7.9 (d, J=8.5 Hz, 2H), 8.5 (s, 1H), 8.8 (s, 1H), 9.1 (s, 1H), 9.6 (t, J=6.5 Hz, 1H)

MS(APCI) M+1=483.1

mp=>260° C.

### COMPOUND EXAMPLE 13

### 4-[4-oxo-6-(3-trifluoromethyl-benzylcarbamoyl)-4H-pyrido[3,4-d]pyrimidin-3-ylmethyl]-benzoic acid

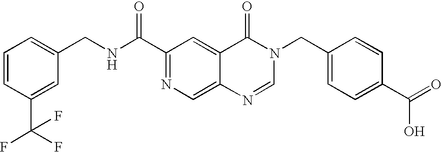

This compound was synthesized as previously described in Compound Example 1, Step (g) using 3-(4-tert-butoxycarbonyl-benzyl)-4-oxo-3,4-dihydro-pyrido[3,5-d]pyrimidine-6-carboxylic acid methyl ester and 3-trifluoromethylbenzylamine in place of 4-methoxybenzylamine.

1H NMR (400 MHz, DMSO-D6) delta (ppm) 4.6 (d, J=6.3 Hz, 2H), 5.3 (s, 2H), 7.5 (d, J=8.3 Hz, 2H), 7.6 (m, 4H), 7.9 (d, J=8.3 Hz, 2H), 8.5 (s, 1H), 8.8 (s, 1H), 9.1 (s, 1H), 9.6 (t, J=6.3 Hz, 1H), 13.0 (bs, 1H)

MS(APCI) M+1=483.1

mp=255.0-256.0° C.

### COMPOUND EXAMPLE 14

### 4-[6-(3,4-difluoro-benzylcarbamoyl)-4-oxo-4H-pyrido[3,4-d]pyrimidin-3-ylmethyl]-benzoic acid

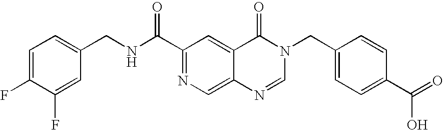

This compound was synthesized as previously described in Compound Example 1, Step (g) using 3-(4-tert-butoxycarbonyl-benzyl)-4-oxo-3,4-dihydro-pyrido[3,5-d]pyriniidine-6-carboxylic acid methyl ester and 3,4-difluorobenzylamine in place of 4-methoxybenzylamine.

1H NMR (400 MHz, DMSO-D6) delta (ppm) 4.5 (d, J=6.3 Hz, 2H), 5.3 (s, 2H), 7.2 (m, J=2.0 Hz, 1H), 7.3 (m, 2H), 7.5 (d, J=8.3 Hz, 2H), 7.9 (d, J=8.3 Hz, 2H), 8.5 (s, 1H), 8.8 (s, 1H), 9.1 (s, 1H), 9.5 (t, J=6.5Hz, 1H), 12.9 (s, 1H)

MS(APCI) M +1=451.1

mp=243.0-244.0° C.

### COMPOUND EXAMPLE 15

### 4-[6-(4-hydroxy-3-methoxy-benzylcarbamoyl)-4-oxo-4H-pyrido[3,4-d]pyrimidin-3-ylmethyl]-benzoic acid

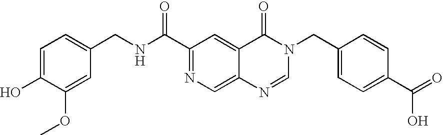

This compound was synthesized as previously described in Example 1, Step (g) using 3-(4-tert-butoxycarbonyl-benzyl)-4-oxo-3,4-dihydro-pyrido[3,5-d]pyrimidine-6-carboxylic acid methyl ester and 4-hydroxy-3-methoxybenzylamine in place of 4-methoxy-benzylamine.

1H NMR (400 MHz, DMSO-D6) delta (ppm) 3.7 (s, 3H), 4.4 (d, J=6.1 Hz, 2H), 5.3 (s, 2H), 6.7 (m, 3H), 6.9 (s, 1H), 7.5 (d, J=8.3 Hz, 2H), 7.9 (d, J=8.3 Hz, 2H), 8.0 (s, 1H), 8.5 (s, 1H), 8.8 (s, 2H), 9.1 (s, 1H), 9.3 (t, 1H), 12.9 (s, 1H)

MS(APCI) M+1=451.1

mp=227.0-228.0° C.

### COMPOUND EXAMPLE 16

### Trans-4-[6-(4-hydroxy-3-methoxy-benzylcarbamoyl)-4-oxo-4H-pyrido[3,4-d]pyrimidin-3-ylmethyl]-cyclohexanecarboxylic acid

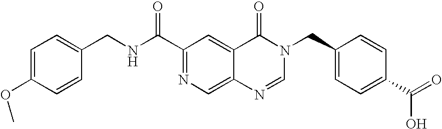

### Step (a): 4-oxo-3,4-dihydro-pyrido[3,4-d]pyridine-6-carboxylic acid methyl ester

A solution of 6-chloro-3H-pyrido[3,4-d]pyrimidin-4-one (20.76 g, 114.3 mmol), in 350 mL of methanol was treated with triethylamine (39.8 mL, 286 mmol), and dppf-PdCl2 (1.87 g, 2.29 mmol), and the mixture was heated at 100° C. under 500 psi of CO for 14 hours. The reaction mixture was cooled to room temperature. The resulting solid was collected by filtration, washed with methanol, washed with ethyl acetate, and dried to give 20.72 g of 4-oxo-3,4-dihydro-pyrido[3,4-d]pyridine-6-carboxylic acid methyl ester as a gray solid (88.3% yield).

1H NMR (400 MHz, DMSO-D6) delta (ppm) 3.9 (s, 3H), 8.3 (s, 1H), 8.5 (s, 1H), 9.1 (s, 1H), 12.8 (bs, 1H)

MS(APCI) M +1=206.1

### Step (b): 4-oxo-3,4-dihydro-pyrido[3,4-d]pyridine-6-carboxylic acid

A suspension of 4-oxo-3,4-dihydro-pyrido[3,4-d]pyridine-6-carboxylic acid methyl ester (2.00 g, 9.75 mmol) in THF/water (60 mL/40 mL) was cooled to 0° C., then treated with LiOH (0.82 g, 19 mmol), and the reaction solution stirred at this temperature for 3 hours. The solution was acidified with 1N HCl, and a precipitated solid was collected by filtration, washed with water and dried to give 1.78 g of 4-oxo-3,4-dihydro-pyrido[3,4-d]pyridine-6-carboxylic acid as gray solid (95.5% yield).

1H NMR (400 MHz, DMSO-D6) delta (ppm) 8.3 (s, 1H), 8.5 (s, 1H,) 9.1 (s, 1H), 12.8 (s, 1H), 13.4 (bs, 1H)

MS(APCI) M−1=190.0

### Step (c): 4-oxo-3,4-dihydro-pyrido[3,4-d]pyridine-6-carboxylic acid 4-methoxy-benzylamide

A suspension of 4-oxo-3,4-dihydro-pyrido[3,4-dlpyridine-6-carboxylic acid (0.80 g, 4.19 mmol) in 30 mL of DMF was treated with EDAC.HCl (1.81 g, 9.42 mmol) and HOBT (1.27 g, 9.42 mmol), then stirred at room temperature for 1 hour. To this mixture was added 4-methoxybenzyl amine (0.86 g, 6.28 mmol) and the reaction mixture stirred overnight at room temperature. The DMF was evaporated, and the resulting residue was diluted with EtOAc and 1 N HCl. The resulting solid was collected by filtration, washed with water, EtOAc, and dried to give 0.58 g of 4-oxo-3,4-dihydro-pyrido[3,4-d]pyridine-6-carboxylic acid 4-methoxy-benzylamide as a gray solid (44.7% yield).

1H NMR (400 MHz, DMSO-D6) delta (ppm) 3.7 (s, 3H), 4.4 (d, J=6.3 Hz, 2H), 6.8 (d, J=8.8 Hz, 2H), 7.3 (d, J=8.5 Hz, 2H), 8.3 (s, 1H), 8.5 (s, 1H), 9.0 (s, 1H), 9.3 (t, J=6.2 Hz, 1H), 12.8 (s, 1H)

MS(APCI)M+1=311.1

### Step (d): Trans-4-[6-(4-methoxy-benzylcarbamoyl)-4-oxo-4H-pyrido[3,4-d]pyrimidin-3-ylmethyl]-cyclohexanecarboxylic acid methyl ester

A solution of 4-oxo-3,4-dihydro-pyrido[3,4-d]pyridine-6-carboxylic acid 4-methoxy-benzylamide (0.55 g, 1.77 mmol) in 7 mL of DMF was treated with cesium carbonate (0.69 g, 2.1 mmol) and the mixture stirred at room temperature for 45 minutes. To this was added 4-methansulfonyloxymethyl-cyclohexanecarboxylic acid methyl ester (0.50 g, 2.1 mmol), and the reaction mixture was heated overnight at 115° C. The reaction mixture was cooled to room temperature, and filtered through a pad of diatomaceous earth, washed with DMF, and the filtrate was evaporated. The resulting residue was diluted with ethyl acetate, washed with 1N HCl, brine, dried over magnesium sulfate, filtered, and evaporated to dryness. This resulting residue was triturated with ether, heated, and allowed to cool to room temperature. The solid was collected by filtration, washed with ether, and dried to give 0.69 g of trans-4-[6-(4-methoxy-benzylcarbamoyl)-4-oxo-4H-pyrido[3,4-d]pyrimidin-3-ylmethyll-cyclohexanecarboxylic acid methyl ester as an off-white solid (83.8% yield).

1H NMR (400 MHz, CHLOROFORM-D) delta (ppm) 1.1 (m, 2H), 1.4 (m, 2H), 1.9 (m, 3H), 2.0 (dd, J=13.9, 3.4 Hz, 2H), 2.3 (m, 1H), 3.6 (s, 3H), 3.8 (s, 3H), 3.9 (d, J=7.1 Hz, 2H), 4.6 (d, J=6.1 Hz, 2H), 6.9 (d, J=8.8 Hz, 2H), 7.3 (m, 2H), 8.1 (s, 1H), 8.3 (t, J=5.9 Hz, 1H), 9.0 (s, 2H)

MS(APCI) M+1=465.3

### Step (e): Trans-4-[6-(4-methoxy-benzylcarbamoyl)-4-oxo-4H-pyrido[3,4-d]pyrimidin-3-ylmethyl]-cyclohexanecarboxylic acid

A solution of trans-4-[6-(4-methoxy-benzylcarbamoyl)-4-oxo-4H-pyrido[3,4-d]pyrimidin-3-ylmethyl]-cyclohexanecarboxylic acid methyl ester (0.19 g, 0.41 mmol) in 40 mL of 6N HCl/MeCN 1:1 was heated at 90° C. for 2 hours, cooled for 30 minutes, then collected by filtration and washed with water. The resulting white solid was dried to give 0.16 g of trans-4-[6-(4-methoxy-benzylcarbamoyl)-4-oxo-4H-pyrido[3,4-d]pyrimidin-3-ylmethyl]-cyclohexanecarboxylic acid.

1H NMR (400 MHz, DMSO-D6) delta (ppm) 1.0 (m, 2H), 1.2 (m, 2H), 1.6 (d, J=12.7 Hz, 2H), 1.7 (m, 1H), 1.9 (d, J=10.7 Hz, 2H), 2.1 (m, 1H), 3.7 (s, 3H), 3.9 (d, J=7.3 Hz, 2H), 4.4 (d, J=6.3 Hz, 2H), 6.8 (d, J=8.8 Hz, 2H), 7.3 (d, J=8.8 Hz, 2H), 8.6 (s, 2H), 9.0 (s, 1H), 9.3 (t, J=6.3 Hz, 1H)

MS(APCI) M+1=451.2

mp=232.0-233.0° C.

Representative invention compounds have been assayed for their abilities to potently inhibit MMP-13 selectively over other MMP enzymes, alleviate pain and inhibit cartilage damage in an arthritic joint, and pass, in sufficient amounts, from the digestive tract into the blood of a mammal and remain in the blood for a time satisfactory for treating a disease as shown below in the biological examples.

An invention compound may be readily identified by one of ordinary skill in the pharmaceutical or medical arts as an inhibitor of MMP-13 by assaying the invention compound for inhibition of MMP-13 as described below in Biological Examples 1 or 2. Such assays are described in detail by Ye et al., in Biochemistry, 1992;31(45):11231-11235, which is incorporated herein by reference. An invention compound may be readily identified by one of ordinary skill in the pharmaceutical or medical arts as an allosteric inhibitor of MMP-13 by assaying the invention compound for inhibition of MMP-13 in the presence of an inhibitor to the catalytic zinc of MMP-13 as described below in Biological Examples 3 or 4.

The assay methods of Biological Examples 1-4 measure the amount by which a test compound reduces the hydrolysis of a thiopeptolide substrate catalyzed by a matrix metalloproteinase enzyme or catalytic domain thereof. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme. The methods described below for the inhibition of MMP-13 may also be adapted and used to determine the ability of the compounds of Formula I to inhibit other matrix metalloproteases such as MMP-1, MMP-2, MMP-3, MMP-7, MMP-8, MMP-9, MMP-12, MMP-14, MMP-17, and the like.

### BIOLOGICAL EXAMPLE 1

Thiopeptolide substrates show virtually no decomposition or hydrolysis at or below neutral pH in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of N-2-hydroxyethylpiperazine-N′-2-ethanesulfonic acid buffer (“HEPES,” pH 7.0), 10 mM CaCl₂, 100 μM thiopeptolide substrate, and 1 mM 5,5′-dithio-bis-(2-nitro-benzoic acid) (DTNB). The thiopeptolide substrate concentration may be varied, for example from 10 to 800 μM to obtain Kₘ and K_{cat} values. The change in absorbance at 405 nm is monitored on a Thermo Max microplate reader (molecular Devices, Menlo Park, Calif.) at room temperature (22° C.). The calculation of the amount of hydrolysis of the thiopeptolide substrate is based on E₄₁₂=13600 M⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Test compounds were evaluated at various concentrations in order to determine their respective IC₅₀ values, the micromolar concentration of compound required to cause a 50% inhibition of catalytic activity of the respective enzyme.

It should be appreciated that the assay buffer used with MMP-3CD was 50 mM N-morpholinoethane sulfonate (“MES”) at pH 6.0 rather than the HEPES buffer at pH 7.0 described above.

### BIOLOGICAL EXAMPLE 2

Some representative compounds of Formula I have been evaluated for their ability to inhibit MMP-13, MMP-1FL, MMP-3CD, MMP-7FL, MMP-8FL, MMP-9FL, MMP-12CD, MMP-14CD, and/or MMP-17CD, wherein FL means full-length enzyme and CD means a catalytic domain of the full-length enzyme. Test compounds can be evaluated at various concentrations in order to determine their respective IC₅₀ values, the micromolar concentration of compound required to cause a 50% inhibition of the hydrolytic activity of the respective enzyme.

The compounds of Formula I, as illustrated by the compounds of Compound Examples 1-16, have been shown to be potent inhibitors of MMP-13 catalytic domain. The compounds of Formula I of Compound Examples 1-16 inhibit MMP-13 catalytic domain as shown below in Biological Table 1 in the column labelled “MMP-13CD IC₅₀ (μM).”

BIOLOGICAL TABLE 1

| | MMP- | | MMP- | | MMP- |
|---|---|---|---|---|---|
| Example | 13CD | Example | 13CD | Example | 13CD IC₅₀ |
| No. | IC₅₀ (μM) | No. | IC₅₀ (μM) | No. | (μM) |
| 1 | 0.0077 | 2 | 0.0013 | 3 | 0.032 |
| 4 | 0.01 | 5 | 0.0050 | 6 | 0.0035 |
| 7 | 0.011 | 8 | 0.0043 | 9 | 0.020 |
| 10 | 0.0029 | 11 | 0.017 | 12 | 0.097 |
| 13 | 0.0026 | 14 | 0.0053 | 15 | 0.020 |
| 16 | 0.043 | | | | |

Certain compounds of Formula I have also been assayed with MMP-1 full-length, MMP-3 catalytic domain, MMP-7 full-length, MMP-8 full-length, MMP-9 full-length, MMP-12 catalytic domain, MMP-14 catalytic domain, and MMP-17 catalytic domain. The IC₅₀'s for the compounds of Compound Examples 1-16 as shown below in Biological Table 2 in the columns labelled “MMP-1FL IC₅₀ (μM),” “MMP-3CD IC₅₀ (μM),” “MMP-7FL IC₅₀ (μM),” “MMP-8FL IC₅₀ (μM),” “MMP-9FL IC₅₀ (μM),” “MMP-12CD IC₅₀ (μM),” “MMP-14CD IC₅₀ (μM),” and “MMP-17CD IC₅₀ (μM),” respectively. In Biological Table 2, the compound example numbers for the Compound Examples are indicated in the column labelled “Ex. No.”

BIOLOGICAL TABLE 2

| | | MMP- | MMP- | MMP- | MMP- | MMP- | MMP- | MMP- |
|---|---|---|---|---|---|---|---|---|
| | MMP- | 3CD | 7FL | 8FL | 9FL | 12CD | 14CD | 17 CD |
| | 1FL IC₅₀ | IC₅₀ | IC₅₀ | IC₅₀ | IC₅₀ | IC₅₀ | IC₅₀ | IC₅₀ |
| Ex. No. | (μM) | (μM) | (μM) | (μM) | (μM) | (μM) | (μM) | (μM) |
| 1 | >100 | 26 | >100 | >100 | >100 | N/a¹ | >100 | >100 |
| 2 | >100 | 14 | >100 | >100 | >100 | N/a | >100 | >100 |
| 3 | >100 | >100 | >100 | >100 | >100 | N/a | >100 | >100 |
| 4 | >100 | >100 | >100 | N/a | >100 | N/a | >100 | >100 |
| 5 | N/a | 81 | N/a | N/a | N/a | >100 | >100 | >100 |
| 6 | N/a | 17 | N/a | N/a | N/a | >100 | >100 | >100 |
| 7 | N/a | >10 | N/a | N/a | N/a | 14 | >30 | >100 |
| 8 | N/a | 33 | N/a | N/a | N/a | >30 | >100 | >100 |
| 9 | N/a | >30 | N/a | N/a | N/a | >30 | >100 | >100 |
| 10 | >100 | 41 | >100 | >100 | >100 | N/a | >100 | >100 |
| 11 | >100 | >100 | >100 | >100 | >100 | N/a | >100 | >100 |
| 12 | >100 | >100 | >100 | >100 | >100 | N/a | >100 | >100 |
| 13 | >100 | 48 | >100 | N/a | >100 | N/a | >100 | >100 |
| 14 | >100 | 42 | >100 | >100 | >100 | N/a | >100 | >100 |
| 15 | >100 | >100 | >30 | >100 | >100 | N/a | >30 | >100 |
| 16 | >100 | >100 | >100 | >100 | >100 | N/a | >100 | >100 |

¹N/a means datum not available

The results shown above in Biological Tables 1 and 2 have established that the compounds of Formula I are potent inhibitors of MMP-13 enzymes, and are especially useful due to their selective inhibition of MMP-13 enzymes over other MMP enzymes. Because of their potent and selective inhibitory activity, the invention compounds are especially useful to treat diseases mediated by an MMP-13 enzyme without side-effects such as musculo-skeletal syndrome (“MSS”) that result from inhibition of other MMP enzymes.

As mentioned above, an invention compound that is an allosteric inhibitor of MMP-13 may be readily identified by assaying the compound for inhibition of MMP-13 according to one of the methods described below in Biological Examples 3 and 4.

### BIOLOGICAL EXAMPLE 3

Fluorigenic peptide-1 substrate based assay for identifying compounds of Formula I as allosteric inhibitors of MMP-13:

Final Assay Conditions:

50 mM HEPES buffer (pH 7.0)
10 mM CaCl₂
10 μM fluorigenic peptide-1 (“FP1”) substrate
0 or 15 mM acetohydroxamic acid (AcNHOH)=1 K_{d}
2% DMSO (with or without inhibitor test compound)
0.5 nM MMP-13CD enzyme
   Stock Solutions:
   1) 10× assay buffer: 500 mM HEPES buffer (pH 7.0) plus 100 mM CaCl₂
   2) 10 mM FP1 substrate: (Mca)-Pro-Leu-Gly-Leu-(Dnp)-Dpa-Ala-Arg-NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Is prepared 10 mM stock by dissolving 5 mg FP1 in 0.457 mL DMSO.
   3) 3 M AcNHOH: Is prepared by adding 4 mL H₂O and 1 mL 10× assay buffer to 2.25 g AcNHOH (Aldrich 15,903-4). Adjusting pH to 7.0 with NaOH. Diluting volume to 10 mL with H₂O. Final solution will contain 3 M AcNHOH, 50 mM HEPES buffer (pH 7.0), and 10 mM CaCl₂.
   4) AcNHOH dilution buffer: 50 mM HEPES buffer (pH 7.0) plus 10 mM CaCl₂
   5) MMP-13CD enzyme: Stock concentration=250 nM.
   6) Enzyme dilution buffer: 50 mM HEPES buffer (pH 7.0), 10 mM CaCl₂, and 0.005% BRIJ 35 detergent (Calbiochem 203728; Protein Grade, 10%)
      Procedure (for One 96-Well Microplate):
      A. Prepared Assay Mixture:

1100 μL 10× assay buffer

[symbol] L 10 mM FP1

55 μL 3 M AcNHOH or 55 μL AcNHOH dilution buffer 8500 μL H₂O

B. Diluted MMP-13CD to 5 nM Working Stock:

22 μL MMP-13CD (250 nM)

1078 μL enzyme dilution buffer

C. Ran Kinetic Assay:

1. Dispense 2 μL inhibitor test sample (in 100% DMSO) into well.
2. Add 88 μL assay mixture and mix well, avoiding bubbles.
3. Initiate reactions with 10 μL of 5 nM MMP-13CD; mix well, avoid bubbles.
4. Immediately measure the kinetics of the reactions at room temperature.

Fluorimeter: Fₘₐₓ Fluorescence Microplate Reader & SOFTMAX PRO Version 1.1 software (Molecular Devices Corporation; Sunnyvale, Calif. 94089).

Protocol menu:

| | |
|---|---|
| excitation: 320 nm | emission: 405 nm |
| run time: 15 min | interval: 29 sec |
| RFU min: −10 | RFU max: 200 |
| Vₘₐₓ points: 32/32 | |

D. Compared % of Control Activity and/or IC₅₀ with Inhibitor Test Compound ±AcNHOH.

Hydrolysis of the fluorigenic peptide-1 substrate, [(Mca)Pro-Leu-Gly-Leu-Dpa-Ala-Arg-NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), is used to screen for MMP-13 catalytic domain (CD) inhibitors. (Dpa may also be abbreviated as “Dnp”.) Reactions (100 μL) contain 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP1, and 0.1 mM to 0.5 nM inhibitor in DMSO (2% final).

After recombinant human MMP-13CD (0.5 nM final) is added to initiate the reaction, the initial velocity of FP1 hydrolysis is determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor is assayed at different concentration values, such as, for example, 100 μM, 10 μM, 1 μM, 100 nM, 10 nM, and 1 nM. Then the inhibitor concentration is plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination is done for experiments done in the presence, and experiments done in the absence, of acetohydroxamic acid. Data are fit to the equation: percent control activity=100/[1+(([I]/IC₅₀)^{slope})], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results may be expressed as an IC₅₀ Ratio (±) ratio, which means a ratio of the IC₅₀ of the inhibitor with MMP-13 and an inhibitor to the catalytic zinc of MMP-13, divided by the IC₅₀ of the inhibitor with MMP-13 without the inhibitor to the catalytic zinc of MMP-13. Invention compounds that are allosteric inhibitors of MMP-13 are expected to have an IC₅₀ Ratio (±) ratio of less than 1, and are expected to be synergistic with the inhibitor to the catalytic zinc of MMP-13 such as, for example, AcNHOH. Invention compounds that are not allosteric inhibitors of MMP-13 will be inactive in the assay or will have an IC₅₀ Ratio (±) of greater than 1, unless otherwise indicated. Results can be confirmed by kinetics experiments that are well known in the biochemical art.

### BIOLOGICAL EXAMPLE 4

Fluorigenic peptide-1 based assay for identifying allosteric inhibitors of matrix metalloproteinase-13 catalytic domain (“MMP-13CD”):

In a manner similar to Biological Example 3, an assay is run wherein 1,10-phenanthroline is substituted for acetohydroxamic acid to identify compounds of Formula ICD.

Testing of the compounds of Compound Examples 1-16 in a method of Biological Example 3 or 4 would establish that the compounds of Formula I, or a pharmaceutically acceptable salt thereof, are allosteric inhibitors of an MMP-13.

Animal models may be used to establish that the instant compounds of Formula I, or a pharmaceutically acceptable salt thereof, would be useful for preventing, treating, and inhibiting damage to extracellular matrix such as cartilage damage, and thus for treating osteoarthritis, for example.

An invention compound having an anti-inflammatory, an analgesic, anti-arthritic, or a cartilage damage inhibiting effect, or any combination of these effects, may be readily identified by one of ordinary skill in the pharmaceutical or medical arts by assaying the invention compound in any number of well known assays for measuring determining the invention compound's effects on cartilage damage, arthritis, inflammation, or pain. These assays include in vitro assays that utilize cartilage samples and in vivo assays in whole animals that measure cartilage degradation, inhibition of inflammation, or pain alleviation.

For example with regard to assaying cartilage damage in vitro, an amount of an invention compound or control vehicle may be administered with a cartilage damaging agent to cartilage, and the cartilage damage inhibiting effects in both tests studied by gross examination or histopathologic examination of the cartilage, or by measurement of biological markers of cartilage damage such as, for example, proteoglycan content or hydroxyproline content. Further, in vivo assays to assay cartilage damage may be performed as follows: an amount of an invention compound or control vehicle may be administered with a cartilage damaging agent to an animal, and the effects of the invention compound being assayed on cartilage in the animal may be evaluated by gross examination or histopathologic examination of the cartilage, by observation of the effects in an acute model on functional limitations of the affected joint that result from cartilage damage, or by measurement of biological markers of cartilage damage such as, for example, proteoglycan content or hydroxyproline content.

Several methods of identifying an invention compound with cartilage damage inhibiting properties are described below. The amount to be administered in an assay is dependent upon the particular assay employed, but in any event is not higher than the well known maximum amount of a compound that the particular assay can effectively accommodate.

Similarly, invention compounds having pain-alleviating properties may be identified using any one of a number of in vivo animal models of pain.

Still similarly, invention compounds having anti-inflammatory properties may be identified using any one of a number of in vivo animal models of inflammation. For example, for an example of inflammation models, see U.S. Pat. No. 6, 329,429, which is incorporated herein by reference.

Still similarly, invention compounds having anti-arthritic properties may be identified using any one of a number of in vivo animal models of arthritis. For example, for an example of arthritis models, see also U.S. Pat. No. 6, 329,429.

Examples of such animal models are described below in Biological Examples 5 and 6.

### BIOLOGICAL EXAMPLE 5

### Monosodium lodoacetate-induced Osteoarthritis in Rat Model of Cartilage Damage (“MIA Rat”):

One end result of the induction of osteoarthritis in this model, as determined by histologic analysis, is the development of an osteoarthritic condition within the affected joint, as characterized by the loss of Toluidine blue staining and formation of osteophytes. Associated with the histologic changes is a concentration-dependent degradation of joint cartilage, as evidenced by affects on hind-paw weight distribution of the limb containing the affected joint, the presence of increased amounts of proteoglycan or hydroxyproline in the joint upon biochemical analysis, or histopathological analysis of the osteoarthritic lesions.

In the MIA Rat model on Day 0, the hind-paw weight differentials between the right arthritic joint and the left healthy joint of male Wistar rats (150 g) were determined with an incapacitance tester, model 2KG (Linton Instrumentation, Norfolk, United Kingdom). The incapacitance tester had a chamber on top with an outwardly sloping front wall that supports a rat's front limbs, and two weight sensing pads, one for each hind paw, that facilitated this determination. Then the rats were anesthetized with isofluorine, and the right, hind leg knee joint was injected with 1.0 mg of mono-iodoacetate (“MIA”) through the infrapatellar ligament. Injection of MIA into the joint resulted in the inhibition of glycolysis and eventual death of surrounding chondrocytes. The rats were further administered either the compound of Compound Example 1 or vehicle (in the instant case, water) daily for 14 days or 28 days. The compound of Compound Example 1 was administered at doses of 1, 3, 10, and 30 milligrams per kilogram of rat per day, but invention compounds may be administered at other doses such as, for example, 60 mg/kg/day, 90-mg/kg/day, or 100 mg/kg/day according to the requirements of the compound being studied. It is well within the level of ordinary skill in the pharmaceutical arts to determine a proper dosage of an invention compound in this model.

Generally, an invention compound may be administered in this model by oral administration, but optionally intravenous administration via an osmotic pump could be employed. After 7 and 14 days for a two-week study, or 7, 14, and 28 days for a four-week study, the hind-paw weight distribution may be determined. Typically, the animals administered vehicle alone placed greater weight on their unaffected left hind paw than on their right hind paw, while animals administered an invention compound showed a more normal (i.e., more like a healthy animal) weight distribution between their hind paws. This change in weight distribution was proportional to the degree of joint cartilage damage. Percent inhibition of a change in hind paw joint function was calculated as the percent change in hind-paw weight distribution for treated animals versus control animals. For example, for a two week study,

Percent inhibition of a change in hind paw weight distribution

$$= \{ 1 - [ \frac{( Δ {W}_{G} )}{( Δ {W}_{C} )} ] \} \times 100$$

wherein: ΔW_{C} is the hind-paw weight differential between the healthy left limb and the arthritic limb of the control animal administered vehicle alone, as measured on Day 14; and

ΔW_{G} is the hind-paw weight differential between the healthy left limb and the arthritic limb of the animal administered an invention compound, as measured on Day 14.

In the present study the compound of Compound Example 1 was administered perorally, and hind-paw weight differentials were determined at both 2 and 4 weeks. Further, in order to detect the presence of erosion of cartilage in the joints, the animals in the above study were sacrificed at 4 weeks, and the presence or absence of cartilage erosion was determined. The proportion of subjects without hind limb erosions was determined via an Exact Sequential Cochran-Armitage Trend test (SAS® Institute, 1999). The Cochran-Armitage Trend test was employed to determine whether the proportion of positive or “Yes” responders increases or decreases with increasing levels of treatment. For this particular study, it was expected that the number of animals without joint erosions increased with increasing dose.

Results:

In MIA at 2-weeks, the compound of Compound Example 1 (i.e., 4-[6-(4-methoxy-benzylcarbamoyl)-4-oxo-4H-pyrido[3,4-d]pyrimidin-3-ylmethyl]-benzoic acid) inhibited in a dose dependent manner cartilage erosion versus vehicle control animals (n=12 rats per group) at doses of 1 mg/kg, 3 mg/kg, 10 mg/kg, and 30 mg/kg, respectively, in rats orally dosed BID. In MIA at 4-weeks, the compound of Compound Example 1 inhibited cartilage erosion in a 2-dimensional sense (i.e., as indicated by surface area of erosion) versus vehicle control animals (n=12 rats per group) by 40.5%, 55.6%, 61.6%, and 32.9% at doses of 1 mg/kg, 3 mg/kg, 10 mg/kg, and 30 mg/kg, respectively, in rats orally dosed BID. Further, in the vehicle control group, all 12 rats exhibited some cartilage erosion in the joint given MIA. However, 5/12, 7/12*, 7/12*, and 4/12 rats in the treatment groups exhibited no cartilage erosion at the doses of 1 mg/kg, 3 mg/kg, 10 mg/kg, and 30 mg/kg, respectively, wherein * means p<0.05 versus vehicle as by the Cochran-Armitage Test adjusted for multiple comparisons.

The MIA Rat data for the compound of Compound Example 1 have established that invention compounds are effective for the inhibition of joint cartilage damage and alleviating joint pain, and thus useful for the treatment of osteoarthritis or rheumatoid arthritis in human, as well as other mammalian joint diseases or disorders mediated by MMP-13. Further, successful treatment following oral administration in the MIA indicates that the invention compounds would be effective for treating other MMP-13 mediated diseases or disorders such as heart failure, cancer metastasis or angiogenesis, and the like. The effectiveness of the compound of Compound Example 1 in the MIA rat model indicates that the invention compounds will have clinically useful effects in preventing and/or treating these diseases or disorders.

Further, a ridit analysis may be used to determine differences in overall erosion severity in a 3-dimensional sense (i.e., both 2-dimensional surface area of erosion and depth of lesion. This analysis takes into account both the erosion grade (0=no erosion, I=erosion extending into the superficial or middle layers, or II=deep layer erosion), and area (small, medium and large, quantified by dividing the area of the largest erosion in each score into thirds) simultaneously. The analysis recognizes that each unit of severity is different, but does not assume a mathematical relationship between units.

Further, in order to measure biochemical or histopathological end points in the MIA Rat model, some of the animals in the above study may be sacrificed, and the amounts of free proteoglycan in both the osteoarthritic right knee joint and the contralateral left knee joint may be determined by biochemical analysis. The amount of free proteoglycan in the contralateral left knee joint provides a baseline value for the amount of free proteoglycan in a healthy joint. The amount of proteoglycan in the osteoarthritic right knee joint in animals administered an invention compound, and the amount of proteoglycan in the osteoarthritic right knee joint in animals administered vehicle alone, are independently compared to the amount of proteoglycan in the contralateral left knee joint. The amounts of proteoglycan lost in the osteoarthritic right knee joints are expressed as percent loss of proteoglycan compared to the contralateral left knee joint control. The percent inhibition of proteoglycan loss, may be calculated as {1-[(proteoglycan loss from joint (%) with vehicle)−(proteoglycan loss from joint (%) with invention compound)]÷(proteoglycan loss from joint (%) with vehicle)}×100. The proteoglycan loss from joint (%) is calculated by conventional means by comparing proteoglycan content of the affected joint to the proteoglycan content of the contralateral joint.

Another animal model for measuring effects of an invention compound on cartilage damage and inflammation and/or pain is described below in Biological Example 6.

### BIOLOGICAL EXAMPLE 6

### Induction of Experimental Osteoarthritis in Rabbit (“EOA in Rabbit”):

Normal rabbits are anaesthetized and anteromedial incisions of the right knees performed. The anterior cruciate ligaments are visualized and sectioned. The wounds are closed and the animals are housed in individual cages, exercised, and fed ad libitum. Rabbits are given either vehicle (water) or an invention compound dosed three times per day with 30-mg/kg/dose or 10-mg/kg/dose. The invention compound may be administered at other doses such as, for example, 3 times 20 mg/kg/day or 3 times 60 mg/kg/day according to the requirements of the invention compound being studied. The rabbits are euthanized 8 weeks after surgery and the proximal end of the tibia and the distal end of the femur are removed from each animal.

Macroscopic Grading

The cartilage changes on the femoral condyles and tibial plateaus are graded separately under a dissecting microscope (Stereozoom, Bausch & Lomb, Rochester, N.Y.). The depth of erosion is graded on a scale of 0 to 4 as follows: grade 0=normal surface; Grade 1=minimal fibrillation or a slight yellowish discoloration of the surface; Grade 2=erosion extending into superficial or middle layers only; Grade 3=erosion extending into deep layers; Grade 4=erosion extending to subchondral bone. The surface area changes are measured and expressed in mm². Representative specimens may also be used for histologic grading (see below).

Histologic Grading

Histologic evaluation is performed on sagittal sections of cartilage from the lesional areas of the femoral condyle and tibial plateau. Serial sections (5 um) are prepared and stained with safranin-O. The severity of OA lesions is graded on a scale of 0-14 by two independent observers using the histologic-histochemical scale of Mankin et al. This scale evaluates the severity of OA lesions based on the loss of safranin-O staining (scale 0-4), cellular changes (scale 0-3), invasion of tidemark by blood vessels (scale 0-1) and structural changes (scale 0-6). On this latter scale, 0 indicates normal cartilage structure and 6 indicates erosion of the cartilage down to the subchondral bone. The scoring system is based on the most severe b [symbol] [symbol] ologic changes in the multiple sections.

Representative specimens of synovial membrane from the medial and lateral knee compartments are dissected from underlying tissues. The specimens are fixed, embedded, and sectioned (5 um) as above, and stained with hematoxylin-eosin. For each compartment, two synovial membrane specimens are examined for scoring purposes and the highest score from each compartment is retained. The average score is calculated and considered as a unit for the whole knee. The severity of synovitis is graded on a scale of 0 to 10 by two independent observers, adding the scores of 3 histologic criteria: synovial lining cell hyperplasia (scale 0-2); villous hyperplasia (scale 0-3); and degree of cellular infiltration by mononuclear and polymorphonuclear cells (scale 0-5): 0 indicates normal structure.

Statistical Analysis

Mean values and SEM is calculated and statistical analysis was done using the Mann-Whitney U-test.

The results of these studies would be expected to show that an invention compound would reduce the size of the lesion on the tibial plateaus, and perhaps the damage in the tibia or on the femoral condyles. In conclusion, these results would show that an invention compound would have significant inhibition effects on the damage to cartilage.

The foregoing EOA rabbit studies would establish that an invention compound is effective for the inhibition of cartilage damage and inflammation and/or alleviating pain, and thus useful for the treatment of osteoarthritis or rheumatoid arthritis in human, and other mammalian diseases or disorders mediated by MMP-13. The effectiveness of an invention compound in this model would indicate that the invention compound would have clinically useful effects in preventing and/or treating these diseases or disorders.

The compound of Compound Example 1 has also been characterized according to its pharmacokinetics properties. Some of those properties are described below in Biological Example 7.

### BIOLOGICAL EXAMPLE 7

A single 5 mg/kg dose of the compound of Compound Example 1 (i.e., 4-[6-(4-methoxy-benzylcarbamoyl)-4-oxo-4H-pyrido[3,4-d]pyrimidin-3-ylmethyl]-benzoic acid) dissolved in 5% N,N-dimethylacetamide/25% propylene glycol/70% 50 mM Tris base was administered intravenously to a group of 3 Sprague-Dawley rats, and the mean clearance rate of the compound, expressed in milliliters per minute per kilogram of rat body weight (“mL/min/kg”), and the compound's half-life, expressed in hours, were determined by conventional means. Further, a single 5 mg/kg oral dose of the compound of Compound Example 1 was administered in a separate experiment to a group of 3 rats, and the total exposure of blood to the compound of Compound Example 1 was determined by conventional means and reported as the area under the time-concentration of compound curve (“AUC”), expressed in nanograms per hour per milliliter (“ng/hr/mL”).

For comparison purposes, a reference compound was separately characterized according to its pharmacokinetics properties except AUC in a similar manner. The reference compound (“Reference Compound 1”) was the compound of Example 188 of PCT International Patent Application Publication number WO 02/064572 A1, which is also described in PCT International Patent Application Publication number WO 02/064080 A2 in Table IVb on page 76, 5^{th} species from the top. The IC₅₀ with MMP-13 for Reference Compound 1 was reported in WO 02/064080 A2 as 0.00074 μM. Reference Compound 1 is named 4-[6-(4-methoxy-benzylcarbamoyl)- 1 -methyl-2,4-dioxo- 1,4-dihydro-2H-pyrido[3,4-d]pyrimidin-3-ylmethyl]-benzoic acid, and has the structure drawn below:

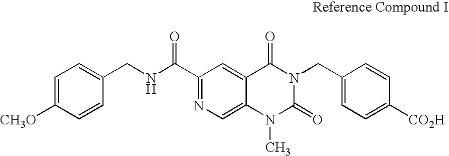

The pharmacokinetics results for the compound of Compound Example 1 and Reference Compound 1 are shown below in Biological Table 3 in the columns labelled “IV CL (mL/min/kg)” for the intravenous clearance rate of the compound from blood, “IV T_{1/2} (hours)” for the intravenous half-life of the compound in blood, and “PO AUC (ng/hr/mL)” for the oral area under the time-concentration of compound curve.

BIOLOGICAL TABLE 3

| Compound | IV CL | IV T_{1/2} | PO AUC |
|---|---|---|---|
| Tested | (mL/min/kg) | (hours) | (ng/hr/mL) |
| Compound of | 7.1 | 2.1 | 3,750 |
| Compound | | | |
| Example 1 | | | |
| Reference | 48.9 | 0.6 | N/D¹ |
| Compound 1 | | | |

¹N/D means not determined

The data in Biological Table 3 show that 4-[6-(4-methoxy-benzylcarbamoyl)-4-oxo-4H-pyrido[3,4-d]pyrimidin-3-ylmethyl]-benzoic acid has phaimacokinetics characteristics that are compatible with its administration as a pharmaceutical in medical and veterinary treatments of mammals suffering from MMP-13 mediated diseases.

Administration according to the invention method of an invention compound as [symbol] armaceutical in medical and veterinary treatments of mammals suffering from MMP-13 mediated diseases listed above is preferably, although not necessarily, accomplished by administering the compound, or a salt thereof, in a pharmaceutical dosage form.

The compounds of Formula I, or a pharmaceutically acceptable salt thereof, can be prepared and administered according to the invention method in a wide variety of oral and parenteral pharmaceutical dosage forms. Thus, the compounds of Formula I, or a pharmaceutically acceptable salt thereof, can be administered by injection, that is, intravenously, intramuscularly, intracutaneously, subcutaneously, intraduodenally, or intraperitoneally. Also, the compounds of Formula I, or a pharmaceutically acceptable salt thereof, can be administered by inhalation, for example, intranasally. Additionally, the compounds of Formula I, or a pharmaceutically acceptable salt thereof, can be administered transdermally. It will be obvious to those skilled in the art that the following dosage forms may comprise as the active component an invention compound. The invention compounds generally are present in a concentration of about 5% to about 95% by weight of the formulation.

For preparing pharmaceutical compositions from the compounds of Formula I, or a pharmaceutically acceptable salt thereof, (i.e., the active component) pharmaceutically acceptable carriers can be either solid or liquid. Solid form preparations are preferred. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or an encapsulating material.

In powders, the carrier is a finely divided solid that is in a mixture with the finely divided active component. Powders suitable for intravenous administration or administration by injection may be lyophilized.

In tablets, the active component is mixed with the carrier having the necessary binding properties in suitable proportions and compacted in the shape and size desired.

The powders and tablets preferably contain from about 5% to about 70%, total, of the active component. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active component with encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogenous mixture is then poured into convenient sized molds, allowed to cool, and thereby to solidify.

Liquid form preparations include solutions, suspensions, and emulsions, for example, water or water propylene glycol solutions. For parenteral injection, liquid preparations can be formulated in solution in aqueous polyethylene glycol solution.

Aqueous solutions suitable for oral use can be prepared by dissolving the active component in water or oil such as migylol, and adding suitable colorants, flavors, stabilizing, and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, falvors, stabilizers, buffers, artificial and natural sweeteners, dispersants, thickeners, solubilizing agents, and the like.

Also included are spray-dried dispersions of an invention compound with a suitable polymer such as hydroxypropylmethyl cellulose (“HPMC”), and hot melt dispersions of an invention compound with a suitable polymer such as polyvinylpyrollidone (“PVP”).

The pharmaceutical preparation is preferably in unit dosage form. In such form, the preparation is subdivided into unit doses containing an appropriate quantity of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.01 to 1000 mg, preferably 1 to 500 mg according to the particular application and the potency of the active components. The composition can, if desired, also contain other compatible therapeutic agents.

In therapeutic use as agents to treat the above-listed diseases, the compounds of Formula I, or a pharmaceutically acceptable salt thereof, are administered at a dose that is effective for treating at least one symptom of the disease or disorder being treated. The initial dosage of about 1 mg/kg to about 100 mg/kg daily of the active component will be effective. A daily dose range of about 25 mg/kg to about 75 mg/kg of the active component is preferred. The dosages, however, may be varied depending upon the requirements of the patient, the severity of the condition being treated, and the particular invention compound being employed in the invention combination. Determination of the proper dosage for a particular situation is within the skill of the art as described above. Typical dosages will be from about 0.1 mg/kg to about 500 mg/kg, and ideally about 25 mg/kg to about 250 mg/kg, such that it will be an amount that is effective to treat the particular disease or disorder being treated.

A preferred composition for dogs comprises an ingestible liquid peroral dosage form selected from the group consisting of a solution, suspension, emulsion, inverse emulsion, elixir, extract, tincture and concentrate, optionally to be added to the drinking water of the dog being treated. Any of these liquid dosage forms, when formulated in accordance with methods well known in the art, can either be administered directly to the dog being treated, or may be added to the drinking water of the dog being treated. The concentrate liquid form, on the other hand, is formulated to be added first to a given amount of water, from which an aliquot amount may be withdrawn for administration directly to the dog or addition to the drinking water of the dog.

A preferred composition provides delayed-, sustained- and/or controlled-release of an invention compound. Such preferred compositions include all such dosage forms which produce ≧40% inhibition of cartilage degradation, and result in a plasma concentration of the active component of at least 3 fold the active component's ED₄₀ for at least 2 hours; preferably for at least 4 hours; preferably for at least 8 hours; more preferably for at least 12 hours; more preferably still for at least 16 hours; even more preferably still for at least 20 hours; and most preferably for at least 24 hours. Preferably, there is included within the above-described dosage forms those which produce ≧40% inhibition of cartilage degradation, and result in a plasma concentration of the active component of at least 5 fold the active component's ED₄₀ for at least 2 hours, preferably for at least 2 hours, preferably for at least 8 hours, more preferably for at least 12 hours, still more preferably for at least 20 hours and most preferably for at least 24 hours. More preferably, there is included the above-described dosage forms which produce ≧50% inhibition of cartilage degradation, and result in a plasma concentration of the active component of at least 5 fold the active component's ED₄₀ for at least 2 hours, preferably for at least 4 hours, preferably for at least 8 hours, more preferably for at least 12 hours, still more preferably for at least 20 hours and most preferably for at least 24 hours.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

All references cited above are hereby incorporated herein by reference.

1. A compound which is 4-[6-(4-methoxy-benzylcarbamoyl)-4-oxo-4H-pyrido[3,4-d]pyrimidin-3-ylmethyl]-benzoic acid, or a pharmaceutically acceptable salt thereof.

2. A compound which is 4-[6-(4-methoxy-benzylcarbamoyl)-4-oxo-4H-pyrido[3,4-d]pyrimidin-3-ylmethyl]-benzoic acid.

3. A pharmaceutical composition, comprising a compound as in claim 1, or a pharmaceutically acceptable salt thereof, together with a pharmaceutically acceptable carrier, diluent, or excipient.

4. A pharmaceutical composition, comprising a compound as in claim 2, or a pharmaceutically acceptable salt thereof, together with a pharmaceutically acceptable carrier, diluent, or excipient.

5. A method of treating osteoarthritis in a mammal in need thereof, comprising administering to the mammal a therapeutically effective amount of a compound as in claim 1, or a pharmaceutically acceptable salt thereof.

6. A method of treating osteoarthritis in a mammal in need thereof, comprising administering to the mammal a therapeutically effective amount of a compound as in claim 2, or a pharmaceutically acceptable salt thereof.
